(19) United States
(12) Patent Application Publication
(10) Pub. No.: US 2020/0216454 A1
(43) Pub. Date: Jul. 9, 2020

(54) BIFUNCTIONAL MOLECULES FOR HER3 DEGRADATION AND METHODS OF USE

(71) Applicant: Dana-Farber Cancer Institute, Inc., Boston, MA (US)

(72) Inventors: Nathanael S. GRAY, Boston, MA (US); James BRADNER, Weston, MA (US); Pasi JANNE, Needham, MA (US); Jaebong JANG, Boston, MA (US); Dennis BUCKLEY, Boston, MA (US); Dennis DOBROVOLSKY, Somerville, MA (US)

(21) Appl. No.: 16/066,149

(22) PCT Filed: Dec. 29, 2016
(86) PCT No.: PCT/US2016/069349

§ 371 Date: Jun. 26, 2018

(51) Int. Cl. C07D 487/04

(52) U.S. Cl. CPC C07D 487/04

(57) ABSTRACT

The invention provides bifunctional compounds which act as protein degradation inducing moieties for a HER family protein, such as Her3. The invention also provides methods for the targeted degradation of a HER family protein through the use of the bifunctional compounds that link a ubiquitin ligase-binding moiety to a ligand that is capable of binding to the HER family protein which can be utilized in the treatment of disorders modulated by a HER family protein.

### STATEMENT OF RELATED APPLICATIONS

This application claims the benefit of U.S. Provisional Patent Application Nos. 62/272,791 filed Dec. 30, 2015 and 62/332,094 filed May 5, 2016. The entirety of these applications are hereby incorporated by reference for all purposes.

### FIELD OF INVENTION

The present invention provides bifunctional molecules for the recruitment of Her3 proteins to E3 ubiquitin ligase proteins for selective degradation.

### BACKGROUND

Ubiquitin-Proteasome Pathway (UPP) is a critical pathway that regulates proteins and degrades misfolded or abnormal proteins. UPP is central to multiple cellular processes, and if defective or imbalanced, leads to pathogenesis of a variety of diseases. The covalent attachment of ubiquitin to specific protein substrates is achieved through the action of E3 ubiquitin ligases. These ligases comprise over 500 different proteins and are categorized into multiple classes defined by the structural element of their E3 functional activity. For example, cereblon (CRBN) interacts with damaged DNA binding protein 1 and forms an E3 ubiquitin ligase complex with cullin-4 in which the proteins recognized by CRBN are ubiquitinated and degraded by proteasomes. Von Hippel-Lindau protein (VHL) is a tumor suppressor protein that forms a complex with elongin-B, elongin-C and cullin-2 which has ubiquitin ligase activity. Various immunomodulatory drugs (IMiDs), such as thalidomide, pomalidomide and lenalidomide, bind to CRBN and modulate CRBN's role in the ubiquitination and degradation of protein factors involved in maintaining regular cellular function.

Harnessing the ubiquitin-proteasome pathway for therapeutic intervention has received significant interest from the scientific community. The publication by Gosink et al. (Proc. Natl. Acad. Sci. USA 1995, 92, 9117-9121) titled “Redirecting the Specificity of Ubiquitination by Modifying Ubiquitin-Conjugating Enzymes” showed proof of concept in vitro that engineered peptides can selectively direct ubiquitination to intracellular proteins. The publication by Nawaz et al. (Proc. Natl. Acad. Sci. U.S.A 1999, 96, 1858-1862) titled “Proteasome-Dependent Degradation of the Human Estrogen Receptor” describes ER degradation as a target for the ubiquitin-proteasome pathway. The publication by Zhou et al. (Mol. Cell 2000, 6, 751-756) titled “Harnessing the Ubiquitination Machinery to Target the Degradation of Specific Cellular Proteins” demonstrated an engineered receptor capable of directing ubiquitination in mammalian and yeast cells.

U.S. Pat. No. 6,306,663 filed in 1999 assigned to Proteinex, Inc., titled “Controlling Protein Levels in Eucaryotic Organisms” appears to be the first patent disclosure of ubiquitinating molecules that incorporate a ubiquitination recognition element and a target protein recognition element.

Perhaps the second general disclosure of such molecules was U.S. Pat. No. 7,041,298 filed in September 2000 by Deshales et al. and granted in May 2006 titled “Proteolysis Targeting Chimeric Pharmaceutical”. The publication by Sakamoto et al. (Proc. Natl. Acad. Sci. USA 2001, 98, 8554-8559) titled “Protacs: Chimeric Molecules That Target Proteins to the Skp1-Cullin-F Box Complex for Ubiquitination and Degradation” describes a “PROTAC” consisting of a small molecule binder of MAP-AP-2 linked to a peptide capable of binding the F-box protein β-TRCP, the disclosure of which is also provided in the corresponding U.S. Pat. No. 7,041,298. The publication by Sakamoto et al. (Mol. Cell. Proteomics 2003, 2, 1350-1358) titled “Development of Protacs to Target Cancer-Promoting Proteins for Ubiquitination and Degradation” describes an analogous PROTAC (PROTAC2) that instead of degrading MAP-AP-2 degrades estrogen and androgen receptors. The publication by Schneekloth et al. (J. Am. Chem. Soc. 2004, 126, 3748-3754) titled “Chemical Genetic Control of Protein Levels: Selective in Vivo Targeted Degradation” describes an analogous degradation agent (PROTAC3) that target the FK506 binding protein (FKBP 12) and by using green fluorescent protein (GFP) imaging, shows that both PROTAC2 and PROTAC3 hit their respective targets with. The publication by Schneekloth et al. (ChemBioChem 2005, 6, 40-46) titled “Chemical Approaches to Controlling Intracellular Protein Degradation” described the state of the field at the time. The publication by Schneekloth et al. (Bioorg. Med. Chem. Lett. 2008, 18, 5904-5908) titled “Targeted Intracellular Protein Degradation Induced by a Small Molecule: En Route to Chemical Proteomics” describes a degradation agent that consist of two small molecules linked by PEG that in vivo degrades the androgen receptor by concurrently binding the androgen receptor and Ubiquitin E3 ligase. WO 2013/170147 filed by Crews et al. titled “Compounds Useful for Promoting Protein Degradation and Methods Using Same” describes compounds comprising a protein degradation moiety covalently bound to a linker, wherein the ClogP of the compound is equal to or higher than 1.5. A review by Buckley et al. (Angew. Chem. Int. Ed. Engl. 2014, 53, 2312-2330) titled “Small-Molecule Control of Intracellular Protein Levels through Modulation of the Ubiquitin Proteasome System” describes a variety of publications. WO 2015/160845 assigned to Arvinas Inc. titled “Imide Based Modulators of Proteolysis and Associated methods of Use” describes the use of degradation compounds including thalidomide to utilize cereblon as the E3 ligase protein. The publication by Lu et al. (Chem. Biol. 2015, 22, 755-763) titled “Hijacking the E3 Ubiquitin Ligase Cereblon to Efficiently Target Brd4” describes thalidomide based degradation compounds useful for degrading BRD4. Additional publications include Bondeson et al. (Nat. Chem. Biol. 2015, 11, 611-617) titled “Catalytic in Vivo Protein Knockdown by Small-Molecule Protacs”; Gustafson et al. (Angewandte Chemie, International Edition in English 2015, 54, 9659-9662) titled “Small-Molecule-Mediated Degradation of the Androgen Receptor through Hydrophobic Tagging”; Buckley et al. (J. Am. Chem. Soc. 2012, 134, 4465-4468) titled “Targeting the Von Hippel-Lindau E3 Ubiquitin Ligase Using Small Molecules to Disrupt the Vhl/Hif-1alpha Interaction”; U.S. 2016/0058872 assigned to Arvinas Inc. titled “Imide Based Modulators of Proteolysis and Associated Methods of Use”; U.S. 2016/0045607 assigned to Arvinas Inc. titled “Estrogen-related Receptor Alpha Based PROTAC Compounds and Associated Methods of Use”; U.S. 2014/0356322 assigned to Yale University, GlaxoSmithKline, and Cambridge Enterprise Limited University of Cambridge titled “Compounds and Methods for the Enhanced Degradation of Targeted Proteins & Other Polypeptides by an E3 Ubiquitin Ligase”; Lai et al. (Angewandte Chemie, International Edition in English 2016, 55, 807-810) titled “Modular Protac Design for the Degradation of Oncogenic Bcr-Abl”; and Toure et al. (Angew. Chem. Int. Ed. 2016, 55, 1966-1973) titled “Small-Molecule Protacs: New Approaches to Protein Degradation”.

It was discovered and reported in 2010 that thalidomide binds to cereblon in (see Ito et al. (Science 2010, 327, 1345-1350) titled “Identification of a Primary Target of Thalidomide Teratogenicity” and Fischer et al. (Nature 2014, 512, 49-53) titled “Structure of the Ddb1-Crbn E3 Ubiquitin Ligase in Complex with Thalidomide”). Itoh et al. also described a small molecule linked to a peptide that utilizes E3 ubiquitin ligase to degrade retinoic acid-binding proteins. (See J. Am. Chem. Soc. 2010, 132, 5820-5826 titled “Protein Knockdown Using Methyl Bestatin-Ligand Hybrid Molecules: Design and Synthesis of Inducers of Ubiquitination-Mediated Degradation of Cellular Retinoic Acid-Binding Proteins”).

A number of bifunctional compounds composed of a target protein-binding moiety and an E3 ubiquitin ligase-binding moiety shown to induce proteasome-mediated degradation of selected proteins are described in WO 2016/077380 and WO 2016/077375 filed by the Dana-Farber Cancer Institute. See also US 2016/0235731 and WO 2016/105518.

There remains a need to provide additional compounds, compositions and methods for the treatment of abnormal cellular proliferation, tumors and cancers.

### SUMMARY

The invention provides novel bifunctional compounds that function to recruit the protein Her3 (receptor tyrosine-protein kinase erbB-3) to a E3 ubiquitin ligase for degradation, and methods of preparation and uses of these compounds. Her3 is a membrane bound protein that is a member of the epidermal growth factor receptor family of kinases. Overexpression of Her3 is implicated in certain breast cancers, lung cancer, head and neck cancer and prostate cancer, among others.

In one embodiment the bifunctional compound is of Formula X:

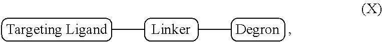

wherein:

the Targeting Ligand binds to Her3 and is selected from:

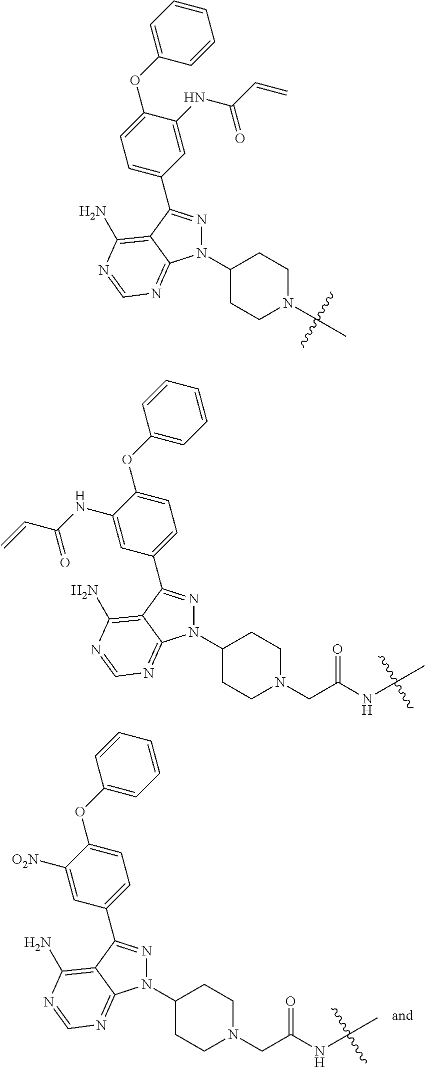 [SMILES: *N1CCC(n2nc(-c3ccc(Oc4ccccc4)c(NC(=O)C=C)c3)c3c(N)ncnc32)CC1 ; *NC(=O)CN1CCC(n2nc(-c3ccc(Oc4ccccc4)c(NC(=O)C=C)c3)c3c(N)ncnc32)CC1 ; *NC(=O)CN1CCC(n2nc(-c3ccc(Oc4ccccc4)c([N+](=O)[O-])c3)c3c(N)ncnc32)CC1]

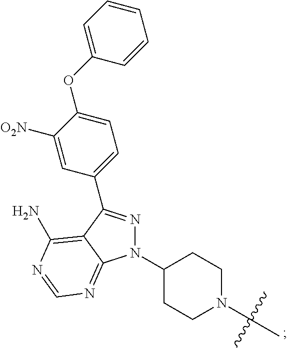 [SMILES: *N1CCC(n2nc(-c3ccc(Oc4ccccc4)c([N+](=O)[O-])c3)c3c(N)ncnc32)CC1]

the Linker is a group that covalently binds to the Targeting Ligand and the Degron; and

the Degron is capable of binding to a ubiquitin ligase, such as an E3 ubiquitin ligase. In some embodiments, the E3 ubiquitin ligase is cereblon or VHL (von Hippel-Lindau).

The invention includes, as examples, bifunctional compounds of Formula Y:

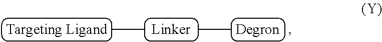

wherein: the

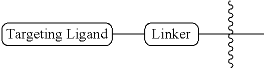

is selected from:

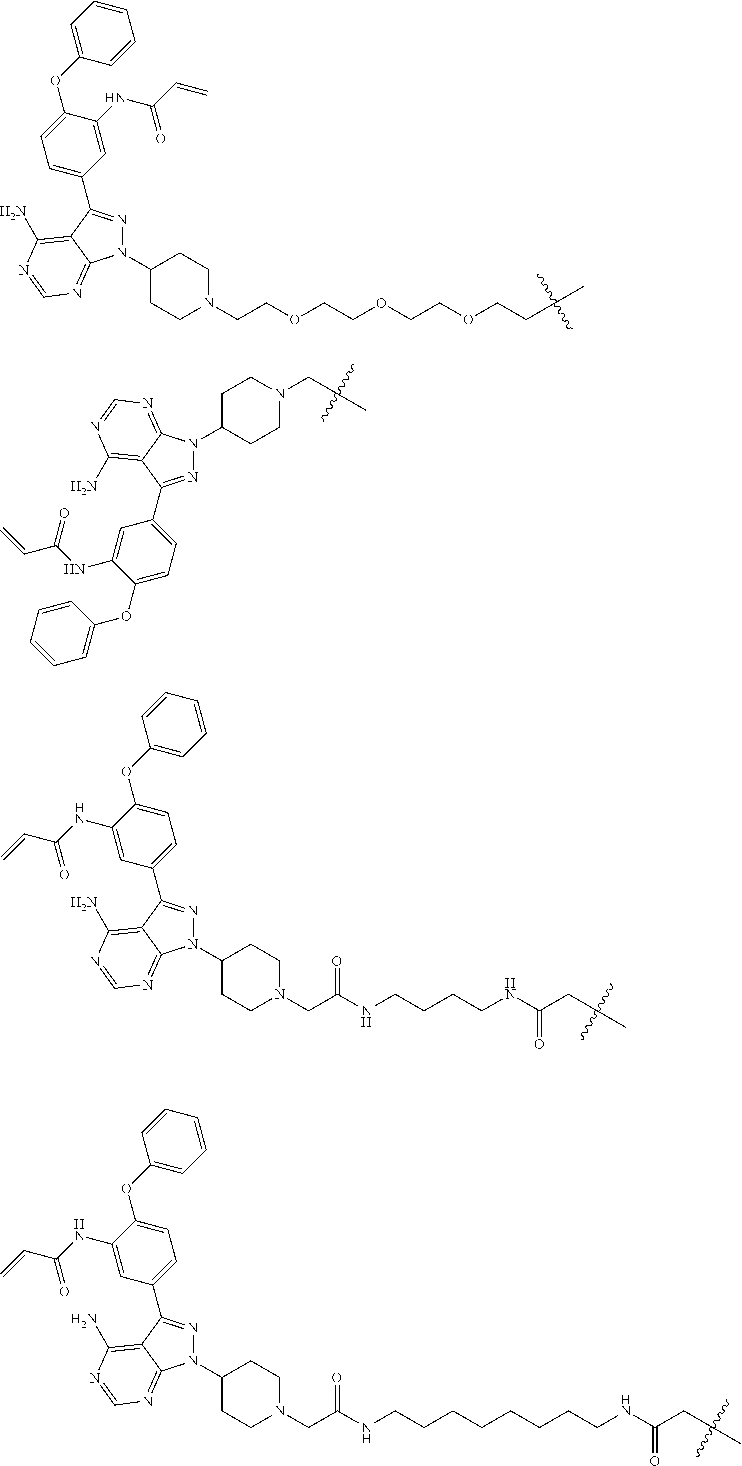 [SMILES: *CCOCCOCCOCCN1CCC(n2nc(-c3ccc(Oc4ccccc4)c(NC(=O)C=C)c3)c3c(N)ncnc32)CC1 ; *CN1CCC(n2nc(-c3ccc(Oc4ccccc4)c(NC(=O)C=C)c3)c3c(N)ncnc32)CC1 ; *CC(=O)NCCCCNC(=O)CN1CCC(n2nc(-c3ccc(Oc4ccccc4)c(NC(=O)C=C)c3)c3c(N)ncnc32)CC1 ; *CC(=O)NCCCCCCCCNC(=O)CN1CCC(n2nc(-c3ccc(Oc4ccccc4)c(NC(=O)C=C)c3)c3c(N)ncnc32)CC1]

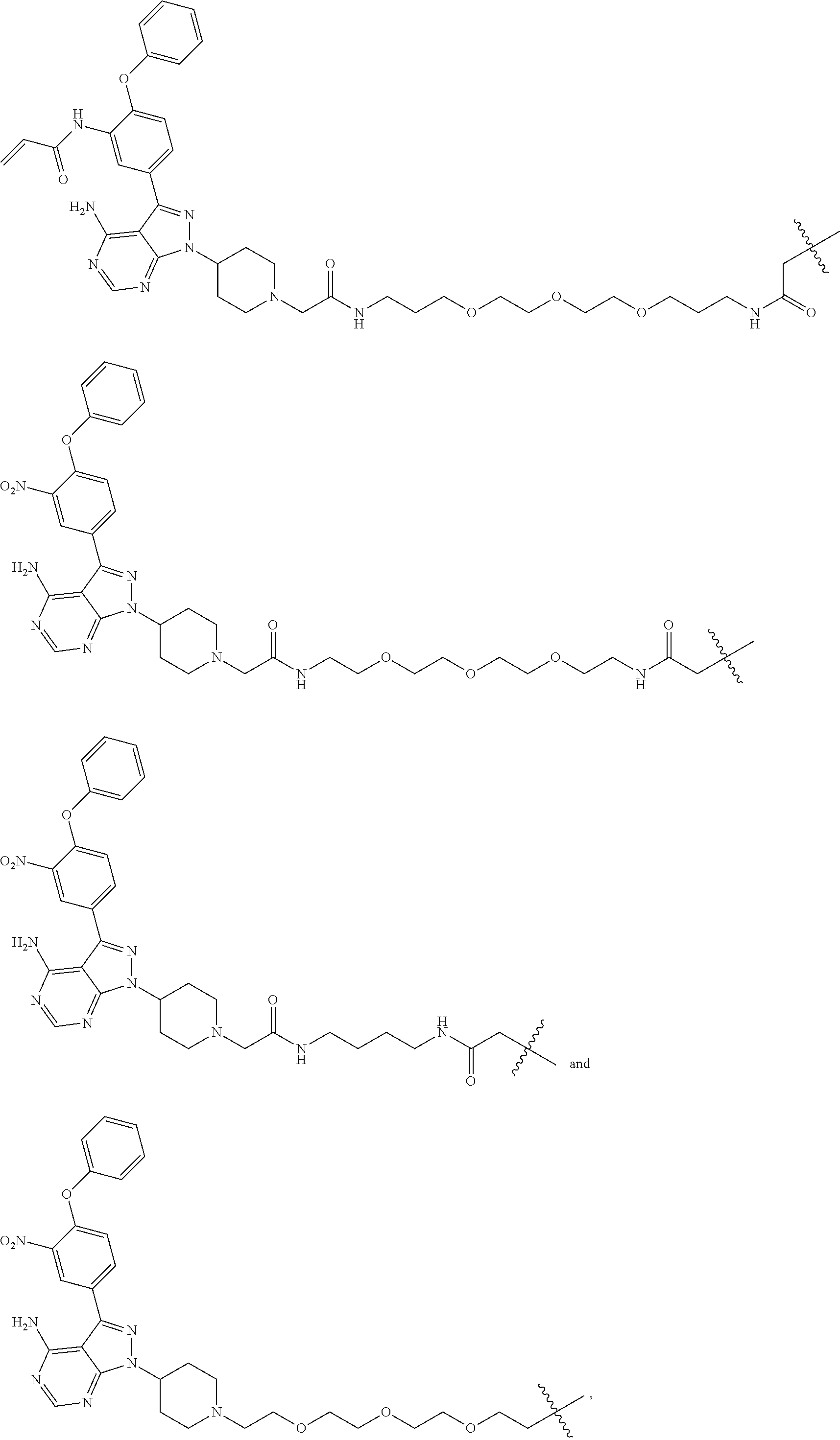 [SMILES: *CC(=O)NCCCOCCOCCOCCCNC(=O)CN1CCC(n2nc(-c3ccc(Oc4ccccc4)c(NC(=O)C=C)c3)c3c(N)ncnc32)CC1 ; *CC(=O)NCCOCCOCCOCCNC(=O)CN1CCC(n2nc(-c3ccc(Oc4ccccc4)c([N+](=O)[O-])c3)c3c(N)ncnc32)CC1 ; *CC(=O)NCCCCNC(=O)CN1CCC(n2nc(-c3ccc(Oc4ccccc4)c([N+](=O)[O-])c3)c3c(N)ncnc32)CC1 ; *CCOCCOCCOCCN1CCC(n2nc(-c3ccc(Oc4ccccc4)c([N+](=O)[O-])c3)c3c(N)ncnc32)CC1]

and the Degron is a group that covalently binds to the Linker and is capable of binding to a ubiquitin ligase, such as an E3 ubiquitin ligase. In one embodiment the E3 ubiquitin ligase is cereblon or VHL.

In one embodiment, the invention includes a bifunctional compound of Formula I:

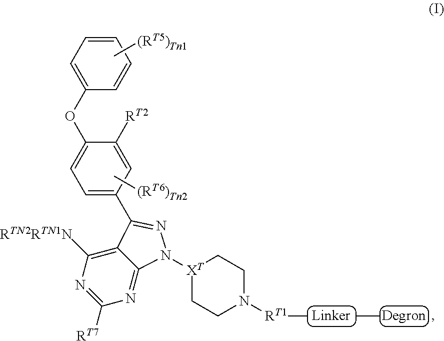

or an enantiomer, diastereomer, stereoisomer, or pharmaceutically acceptable salt thereof, wherein:

X^{T}, Tn1, Tn2, R^{T1}, R^{T2}, R^{T5}, R^{T6}, R^{T7}, R^{TN1}, and R^{TN2} are each as defined herein;

the Linker is a group that covalently binds to R^{T1} and the Degron;

the Degron is capable of binding to a ubiquitin ligase, such as an E3 ubiquitin ligase; and

the Targeting Ligand is capable of binding to a HER family protein. In one embodiment the E3 ubiquitin ligase is cereblon. In one embodiment the HER family protein is Her3.

In one embodiment the Degron is of Formula D1 or D2:

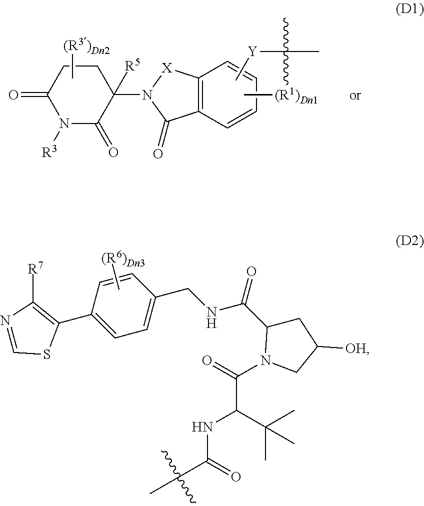

or an enantiomer, diastereomer, or stereoisomer thereof, wherein X, Y, R¹, R³, R^{3′}, R⁵, R⁶, R⁷, Dn1, Dn2, and Dn3 are each as defined herein.

In one embodiment the Linker is of Formula L0:

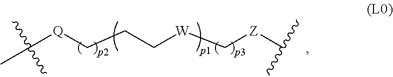

or an enantiomer, diastereomer, or stereoisomer thereof, wherein p1, p2, p3, W, Q, and Z are each as defined herein, the Linker is covalently bonded to a Degron with the

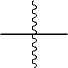

next to Q, and covalently bonded to a Targeting Ligand with the

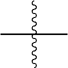

next to Z.

The invention also provides a pharmaceutical composition comprising a therapeutically effective amount of the described bifunctional compound of the application, or an enantiomer, diastereomer, stereoisomer, or pharmaceutically acceptable salt thereof, and a pharmaceutically acceptable carrier.

The invention also provides a method for modulating the amount of a HER family protein by administering a therapeutically effective amount of a bifunctional compound or a pharmaceutical composition of the invention to a subject in need thereof. In one embodiment the targeted proteins is a Her protein. In a further embodiment, the targeted protein is Her3. In an additional embodiment, the application provides a method for decreasing the amount of a targeted protein by administering a therapeutically effective amount of a bifunctional compound or a pharmaceutical composition of the application to a subject in need thereof.

The invention also provides a method for treating a disease or condition which is modulated by a targeted protein by administering a therapeutically effective amount of a bifunctional compound or a pharmaceutical composition of the application to a subject in need thereof. In one embodiment the disease or condition is a cancer modulated by a targeted protein. In a further embodiment the cancer is modulated by a HER family protein. In yet a further embodiment, the cancer is modulated by the Her3 protein.

The invention also provides a bifunctional compound or a pharmaceutical composition of the application for use in treating a disease or condition which is modulated by a targeted protein or for modulating the amount of a targeted protein. In one embodiment, the bifunctional compound or the pharmaceutical composition is used to treat a cancer that is modulated by a targeted protein. In a further embodiment the cancer is modulated by a HER family protein. In yet a further embodiment, the cancer is modulated by the Her3 protein. In one embodiment, the bifunctional compound or the pharmaceutical composition is used to decrease the amount of a HER family protein. In a further embodiment, the HER family protein is Her3.

The invention also provides the use of a bifunctional compound or a pharmaceutical composition of the application for treating a disease or condition which is modulated by a targeted protein or for modulating the amount of a targeted protein. In one embodiment, the use of a bifunctional compound or the pharmaceutical composition is for treating a cancer modulated by a targeted protein. In a further embodiment, the targeted protein in a HER family protein. In yet a further embodiment, the HER family protein is Her3. In one embodiment, the use of a bifunctional compound or the pharmaceutical composition is for decreasing the amount of a HER family protein. In a further embodiment, the HER family protein is Her3.

The invention also provides the use of a bifunctional compound or a pharmaceutical composition of the application in the manufacture of a medicament for treating a disease or condition which is modulated by a targeted protein or for modulating the amount of a targeted protein. In one embodiment, the use of a bifunctional compound or a pharmaceutical composition in the manufacture of a medicament is for treating a cancer modulated by a targeted protein. In a further embodiment, the targeted protein is a HER family protein. In a further embodiment, the HER family protein is Her3. In one embodiment, the use of a bifunctional compound or a pharmaceutical composition in the manufacture of a medicament is for decreasing the amount of a HER family protein. In a further embodiment the HER family protein is Her3.

The compounds and methods of the invention address unmet needs in the treatment of diseases or disorders in which pathogenic or oncogenic endogenous proteins play a role, such as cancer. In one embodiment the pathogenic or oncogenic endogenous proteins are a HER family protein. In a further embodiment the HER family protein is Her3.

Unless otherwise defined, all technical and scientific terms used herein have the same meaning as commonly understood by one of ordinary skill in the art to which this application belongs. In the specification, the singular forms also include the plural unless the context clearly dictates otherwise. Although methods and materials similar or equivalent to those described herein can be used in the practice or testing of the invention, suitable methods and materials are described below. All publications, patent applications, patents, and other references mentioned herein are incorporated by reference. The references cited herein are not admitted to be prior art to the application. In the case of conflict, the present specification, including definitions, will control. In addition, the materials, methods, and examples are illustrative only and are not intended to be limiting. Other features and advantages of the invention will be apparent from the following detailed description and claims.

### DETAILED DESCRIPTION

### Her3 Target Protein

Her3 (ErbB3) is a trans-membrane receptor tyrosine kinase that becomes deregulated in many cancers such as breast, ovarian, and non-small cell lung cancer. Her3 is a member of the HER family of receptor tyrosine kinases that also includes EGFR (Her1), Her2, and Her4, any of which can be targeted with the present invention. The HER family of receptors monitor extracellular levels of growth factors and use this information in conjunction with other signals that allow the cell to decide when to proliferate. HER proteins function in pairs by binding to each other. For example EGFR and Her2 each pair with Her3 to make an active signaling dimer. Unlike EGFR, Her2, and Her4, Her3 has extremely low kinase activity and accordingly is considered “undruggable.”

The majority of clinical research on targeting Her3 has centered on the use of monoclonal antibodies. The publication by Zhang et al. (Acta Biochim Biophys Sin 2015, 48, 39-48) titled “Her3/ErbB3, an emerging cancer therapeutic target” and the publication by Ma et al. (Molecular Cancer 2014, 13, 105) titled “Targeting of ErbB3 receptor to overcome resistance in cancer treatment” discusses recent clinical developments of anti-Her3 monoclonal antibodies. One fully humanized anti-Her3 monoclonal antibody in clinical trials is MM-121 (seribantumab) developed by Merrimack Pharmaceuticals/Sanofi Aventis (PCT WO2008/100624). This antibody has been extensively studied and is currently in Phase 1 and Phase 2 clinical trials for various types of cancers, including breast, ovarian, and non-small cell lung cancer for use in combination with chemotherapy and tyrosine kinase inhibitors (examples of clinical trials include NCT01209195, NCT01451632, NCT01421472, and NCT00994123). A second fully humanized anti-Her3 monoclonal antibody in clinical trials is AMG-888 (Patritumab). Developed by Daiichi Sankyo Inc. (WO2007/077028), AMG-888 is currently being tested in a Phase 3 clinical trial (NCT02134015) where subjects are given AMG-888 in combination with Erlotinib. A Phase 1 clinical trial (NCT00730470) has also been completed for patients with advanced solid tumors and a Phase 1b/2 study is ongoing investigating AMG-888 in combination with the anti-Her2 monoclonal antibody trastuzumab and the chemotherapeutic paclitaxel in patients newly diagnosed with metastatic breast cancer. Other clinical anti-Her3 clinical candidates include RG7116 (lumretuzumab, RO-5479599) by Hoffmann-La Roche, LJM716 developed by Novartis International AG, GSK2849330 by GlaxoSmithKline PLC, and MIM0111 developed by Merrimack Pharmaceuticals. Disclosures for anti-Her3 monoclonal antibodies include WO1997/35885 to Genentech Inc., WO2007/077028 to U3 Pharma, WO2008/100624 to Merrimack Pharmaceuticals, WO2011/136911 to Aveo Pharmaceuticals, WO2012/019024 to Immunogen, WO2012/022814 to Novartis, WO2015/048008 to Medlmmune, WO2016/177664 to Gamamabs Pharma, and US 20160311923 to Sorrento Therapeutics.” Despite this work, to date no Her3-targeted therapy has been FDA approved.

Small molecule inhibitors of Her3 have been identified. Pyrazolo[3,4-d]pyrimidin-4-amine based compounds for targeting kinase proteins are disclosed in WO 2001/019829 and WO 2002/080926 both of which are assigned to BASF AG. In a paper titled “Pharmacological targeting pseudokinase Her3” (Xie et al., Nature Chemical Biology, 2014, 10(12), 1006-1012), these pyrazolo[3,4-d]pyrimidin-4-amine based compounds, including a lead compound TX1-85-1 that had an IC₅₀ value of 23 nM at Her3, were shown to be targeting Her3. Xie et al. also disclosed an adamantine-containing bifunctional compound, TX2-121-1 with an IC₅₀ of 49 nM at Her3. Lim et al. in a paper titled “Development of small molecules targeting the pseudokinase Her3” (Bioorg Med Chem Lett. 2015, 25, 3382) disclosed a series of compounds based on TX1-85-1 and TX2-121-1 that exhibited varying levels of inhibition at Her3 with the best compounds having adamantine functional groups.

### Compounds of the Application

The invention provides bifunctional compounds having utility as modulators of ubiquitination and proteosomal degradation of targeted proteins, especially compounds comprising a moiety capable of binding to a polypeptide or a protein that is degraded and/or otherwise inhibited by the bifunctional compounds of the invention. In particular, the invention is directed to compounds which contain a small-molecule moiety that is capable of binding to an E3 ubiquitin ligase, such as cereblon, and a ligand that is capable of binding to a target protein, in such a way that the target protein is placed in proximity to the ubiquitin ligase to effect degradation (and/or inhibition) of that protein. In one embodiment, the small molecule moiety has a molecular weight below 2,000, 1,000, 500, or 200 Daltons. In one embodiment, the small molecule moiety is a thalidomide-like moiety. In certain embodiments, the E3 ubiquitin ligase is cereblon or VHL.

In one embodiment, the invention provides a bifunctional compound of Formula X:

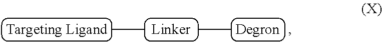

wherein:

the Targeting Ligand is selected from:

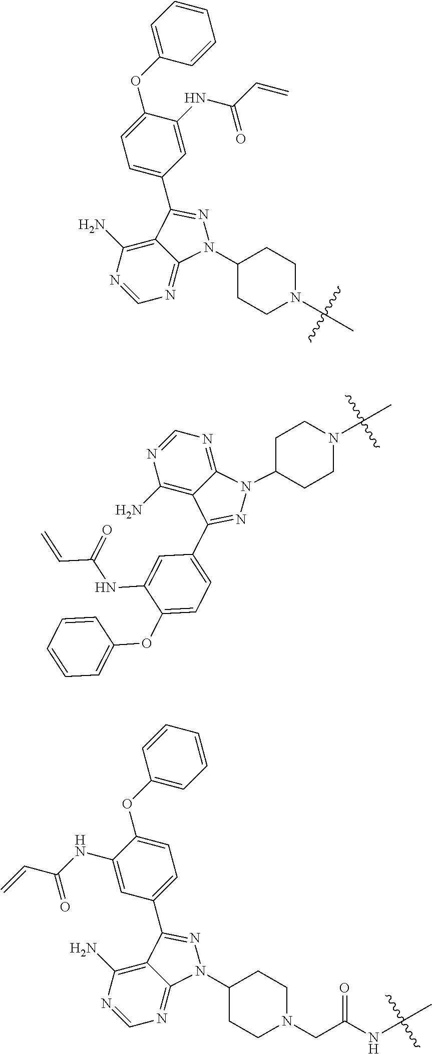 [SMILES: *N1CCC(n2nc(-c3ccc(Oc4ccccc4)c(NC(=O)C=C)c3)c3c(N)ncnc32)CC1 ; *N1CCC(n2nc(-c3ccc(Oc4ccccc4)c(NC(=O)C=C)c3)c3c(N)ncnc32)CC1 ; *NC(=O)CN1CCC(n2nc(-c3ccc(Oc4ccccc4)c(NC(=O)C=C)c3)c3c(N)ncnc32)CC1]

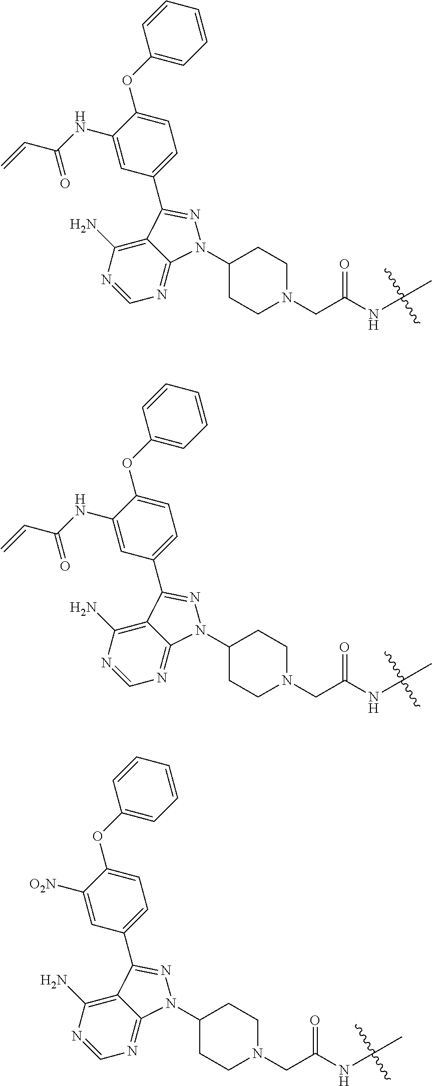 [SMILES: *NC(=O)CN1CCC(n2nc(-c3ccc(Oc4ccccc4)c(NC(=O)C=C)c3)c3c(N)ncnc32)CC1 ; *NC(=O)CN1CCC(n2nc(-c3ccc(Oc4ccccc4)c(NC(=O)C=C)c3)c3c(N)ncnc32)CC1 ; *NC(=O)CN1CCC(n2nc(-c3ccc(Oc4ccccc4)c([N+](=O)[O-])c3)c3c(N)ncnc32)CC1]

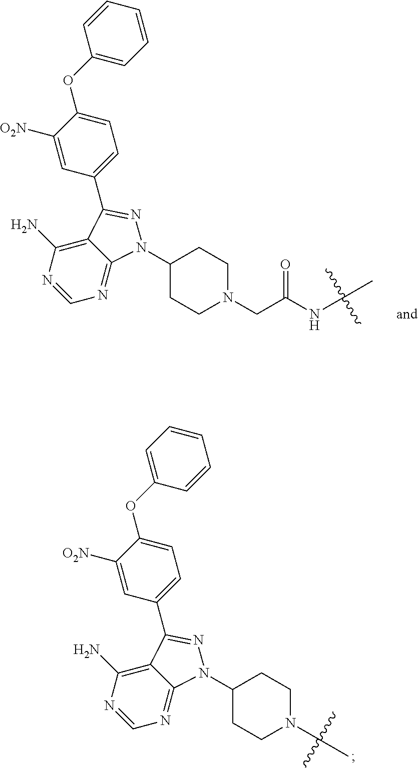 [SMILES: *NC(=O)CN1CCC(n2nc(-c3ccc(Oc4ccccc4)c([N+](=O)[O-])c3)c3c(N)ncnc32)CC1 ; *N1CCC(n2nc(-c3ccc(Oc4ccccc4)c([N+](=O)[O-])c3)c3c(N)ncnc32)CC1]

the Linker is a group that covalently binds to the Targeting Ligand and the Degron; and

the Degron is capable of binding to a ubiquitin ligase, such as an E3 ubiquitin ligase. In certain embodiments the E3 ubiquitin ligase is cereblon or VHL.

In one embodiment, the invention provides a bifunctional compound of Formula Y:

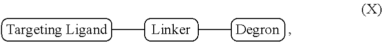

wherein

the

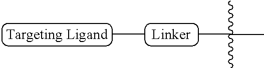

is selected from:

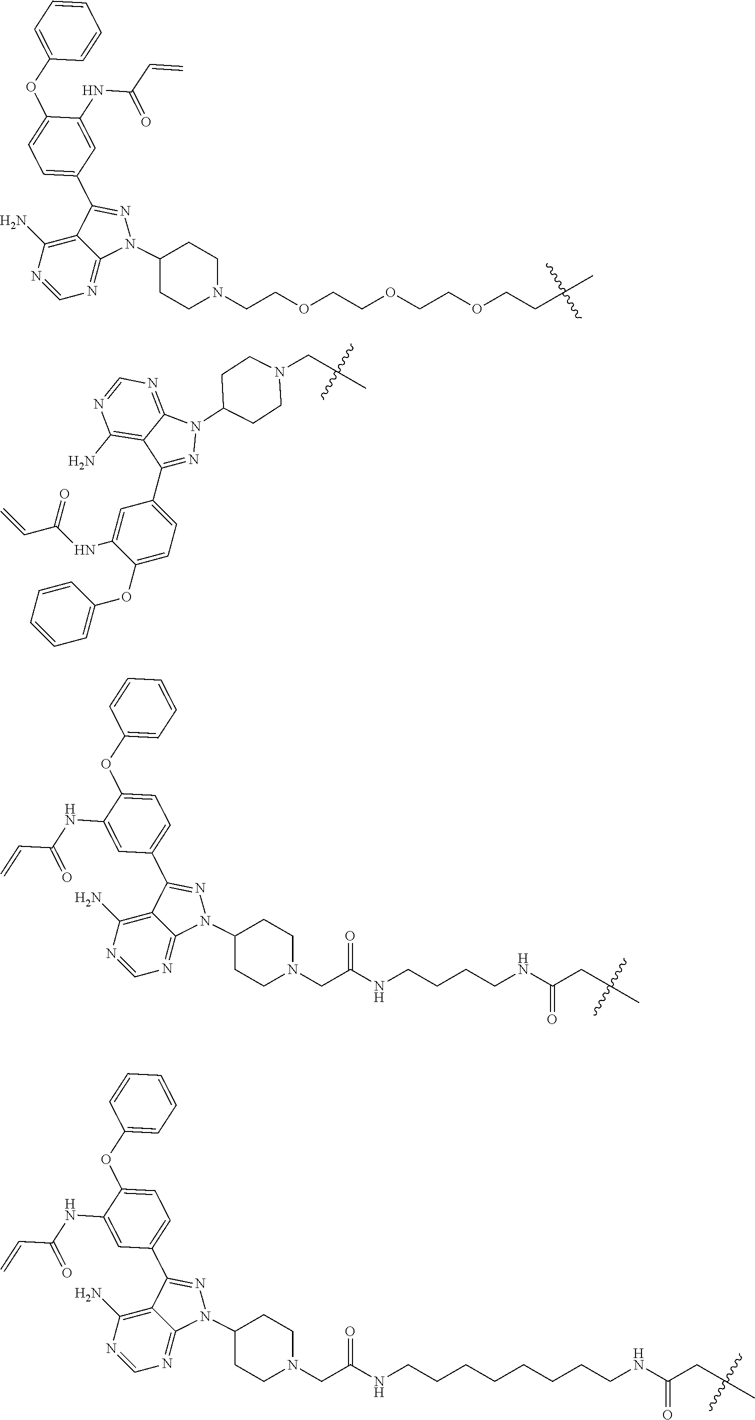

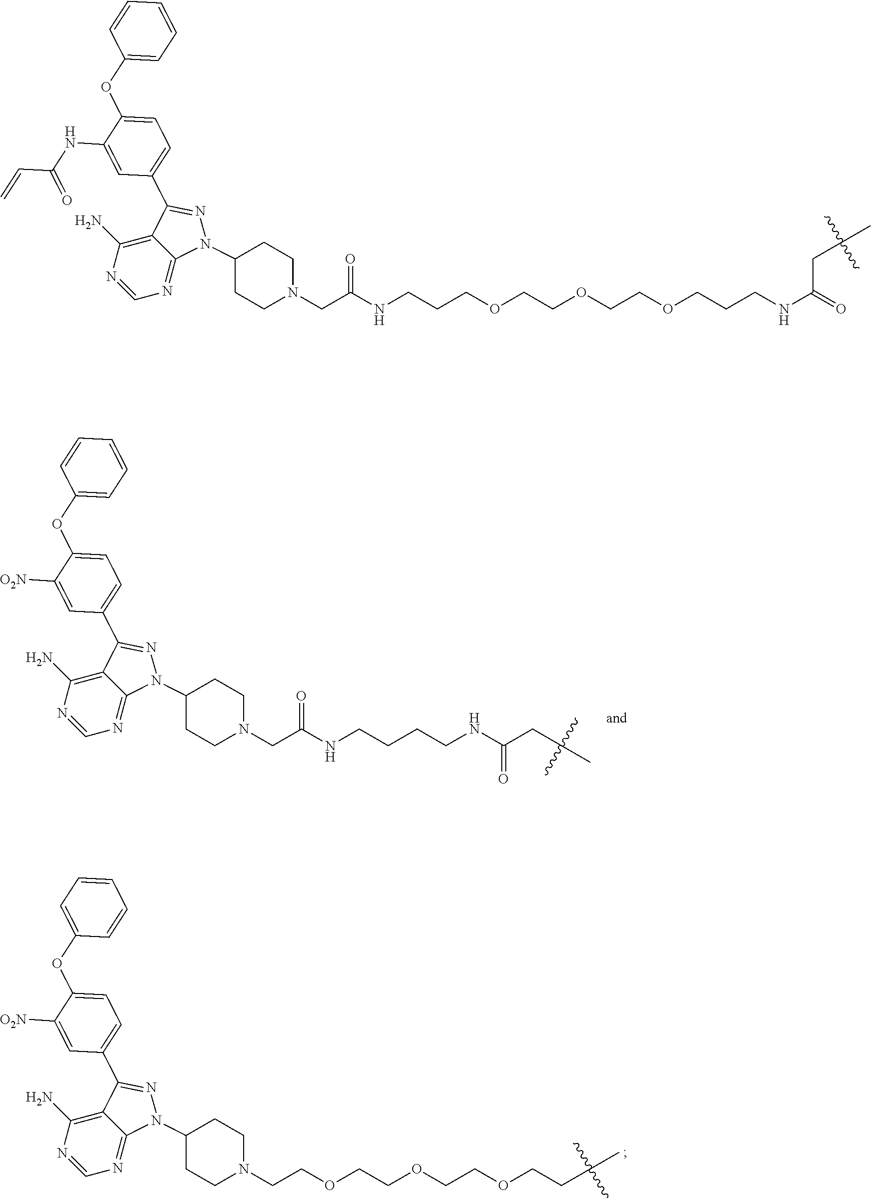 [SMILES: *CC(=O)NCCCOCCOCCOCCCNC(=O)CN1CCC(n2nc(-c3ccc(Oc4ccccc4)c(NC(=O)C=C)c3)c3c(N)ncnc32)CC1 ; *CC(=O)NCCCCNC(=O)CN1CCC(n2nc(-c3ccc(Oc4ccccc4)c([N+](=O)[O-])c3)c3c(N)ncnc32)CC1 ; *CCOCCOCCOCCN1CCC(n2nc(-c3ccc(Oc4ccccc4)c([N+](=O)[O-])c3)c3c(N)ncnc32)CC1]

and

the Degron is a group that covalently binds to the Linker and is capable of binding to a ubiquitin ligase, such as an E3 ubiquitin ligase. In one embodiment the E3 ubiquitin ligase is cereblon.

In one embodiment, the invention provides a compound of Formula I:

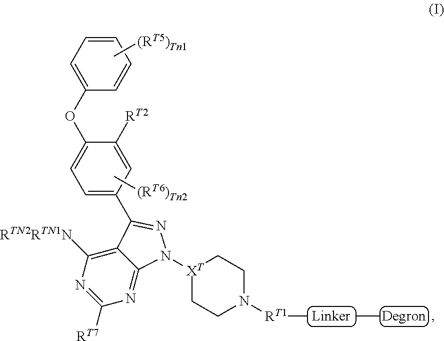

or an enantiomer, diastereomer, stereoisomer, or pharmaceutically acceptable salt thereof, wherein:

X^{T}, Tn1, Tn2, R^{T1}, R^{T2}, R^{T5}, R^{T6}, R^{T7}, R^{TN1}, and R^{TN2} are each as defined herein;

the Linker is a group that covalently binds to R^{T1} and the Degron;

the Degron is capable of binding to a ubiquitin ligase, such as an E3 ubiquitin ligase; and

the Targeting Ligand is capable of binding to a HER family protein. In one embodiment the HER family protein is Her3. In certain embodiments the E3 ubiquitin ligase is cereblon or VHL.

### Targeting Ligand

Targeting Ligand (TL) (or target protein moiety or target protein ligand or ligand) is a small molecule which is capable of binding to a target protein of interest, such as a HER family protein. These species can be found in “Pharmacological targeting pseudokinase Her3” (Xie et al., Nature Chemical Biology, 2014, 10(12), 1006-1012 and “Development of small molecules targeting the pseudokinase Her3” (Lim et al., Bioorg Med Chem Lett. 2015, 25, 3382). In one embodiment the HER family protein is Her3.

In one embodiment, a Targeting Ligand is a compound of Formula TL-I:

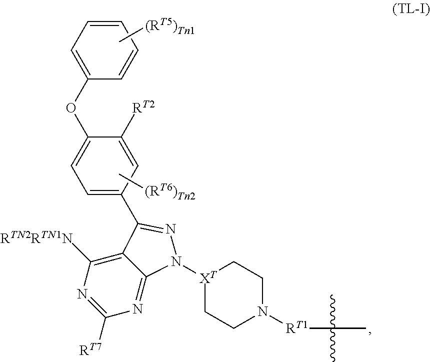

or an enantiomer, diastereomer, stereoisomer, or pharmaceutically acceptable salt thereof, wherein:

X^{T} is N or CH;

R^{T1} is absent, (CH₂)₀₋₃C(O)NH, or (CH₂)₀₋₃NHC(O);

R^{T2} is NO₂ or NH₂;

Tn1 is 0, 1, 2, 3, 4, or 5;

each R^{T5} is independently OH, halogen, CN, C₁-C₄ alkyl, C₁-C₄ alkyl substituted with halogen, C₁-C₄ alkoxy, or C₁-C₄ alkoxy substituted with halogen;

Tn2 is 0, 1, 2, or 3;

each R^{T6} is independently OH, halogen, CN, C₁-C₄ alkyl, C₁-C₄ alkyl substituted with halogen, C₁-C₄ alkoxy, or C₁-C₄ alkoxy substituted with halogen;

R^{T7} is H or C₁-C₄ alkyl; and

R^{TN1} and R^{TN2} are each independently H or C₁-C₄ alkyl,

wherein the Targeting Ligand is bonded to a Linker via the

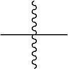

next to R^{T1}.

In one embodiment, X^{T} is N.

In one embodiment, X^{T} is CH.

In one embodiment, R^{T1} is absent.

In one embodiment, R^{T1} is (CH₂)₀₋₃C(O)NH, including but not limited to C(O)NH, (CH₂)C(O)NH, (CH₂)₂C(O)NH, or (CH₂)₃C(O)NH. In one embodiment, R^{T1} is (CH₂)C(O)NH.

In one embodiment, R^{T1} is (CH₂)₀₋₃NHC(O), including but not limited to NHC(O), (CH₂)NHC(O), (CH₂)₂NHC(O), or (CH₂)₃NHC(O).

In one embodiment, R^{T2} is NO₂.

In one embodiment, R^{T2} is NH₂.

In one embodiment, Tn1 is 0, 1, or 2.

In one embodiment, Tn1 is 0.

In one embodiment, at least one R^{T5} is OH, halogen, or CN. In one embodiment, at least one R^{T5} is halogen. In one embodiment, at least one R^{T5} is F or Cl.

In one embodiment, at least one R^{T5} is C₁-C₄ alkyl, including but not limited to methyl, ethyl, n-propyl, i-propyl, n-butyl, i-butyl, or t-butyl or C₁-C₄ alkyl, including but not limited to methyl, ethyl, n-propyl, i-propyl, n-butyl, i-butyl, or t-butyl substituted with halogen. In one embodiment, at least one R^{T5} is C₁-C₄ alkyl, including but not limited to methyl, ethyl, n-propyl, i-propyl, n-butyl, i-butyl, or t-butyl.

In one embodiment, at least one R^{T5} is C₁-C₄ alkoxy, including but not limited to methoxy, ethoxy, n-propoxy, i-propoxy, n-butoxy, i-butoxy, or t-butoxy or C₁-C₄ alkoxy, including but not limited to methoxy, ethoxy, n-propoxy, i-propoxy, n-butoxy, i-butoxy, or t-butoxy substituted with halogen.

In one embodiment, Tn2 is 0 or 1.

In one embodiment, Tn2 is 0.

In one embodiment, at least one R^{T6} is OH, halogen, or CN. In one embodiment, at least one R^{T6} is halogen. In one embodiment, at least one R^{T6} is F or C₁.

In one embodiment, at least one R^{T6} is C₁-C₄ alkyl, including but not limited to methyl, ethyl, n-propyl, i-propyl, n-butyl, i-butyl, or t-butyl or C₁-C₄ alkyl, including but not limited to methyl, ethyl, n-propyl, i-propyl, n-butyl, i-butyl, or t-butyl) substituted with halogen. In one embodiment, at least one R^{T6} is C₁-C₄ alkyl, including but not limited to methyl, ethyl, n-propyl, i-propyl, n-butyl, i-butyl, or t-butyl.

In one embodiment, at least one R^{T6} is C₁-C₄ alkoxy, including but not limited to methoxy, ethoxy, n-propoxy, i-propoxy, n-butoxy, i-butoxy, or t-butoxy or C₁-C₄ alkoxy, including but not limited to methoxy, ethoxy, n-propoxy, i-propoxy, n-butoxy, i-butoxy, or t-butoxy substituted with halogen.

In one embodiment, R^{T7} is H.

In one embodiment, R^{T7} is C₁-C₄ alkyl, including but not limited to methyl, ethyl, n-propyl, i-propyl, n-butyl, i-butyl, or t-butyl.

In one embodiment, R^{TN1} and R^{TN2} are each H.

In one embodiment, one of R^{TN1} and R^{TN2} is H, and the other C₁-C₄ alkyl, including but not limited to methyl, ethyl, n-propyl, i-propyl, n-butyl, i-butyl, or t-butyl.

In one embodiment, R^{TN1} and R^{TN2} are each independently C₁-C₄ alkyl, including but not limited to methyl, ethyl, n-propyl, i-propyl, n-butyl, i-butyl, or t-butyl.

Any of the groups described herein for any of X^{T}, Tn1, Tn2, R^{T1}, R^{T2}, R^{T5}, R^{T6}, R^{T}7, R^{TN1}, and R^{TN2} can be combined with any of the groups described herein for one or more of the remainder of X^{T}, Tn1, Tn2, R^{T1}, R^{T2}, R^{T5}, R^{T6}, R^{T7}, R^{TN1}, and R^{TN2}, and may further be combined with any of the groups described herein for the Linker.
(1) In one embodiment, X^{T} is CH and R^{T1} is (CH₂)₀₋₃C(O)NH. In one embodiment, X^{T} is CH and R^{T1} is (CH₂)C(O)NH.
(2) In one embodiment, X^{T} is CH and R^{T1} is absent.
(3) In one embodiment, X^{T} is CH and R^{T1} is (CH₂)₀₋₃NHC(O). In one embodiment, X^{T} is CH and R^{T1} is (CH₂)NHC(O).
(4) In one embodiment, X^{T} is CH and R^{T2} is NO₂.
(5) In one embodiment, X^{T} is CH; R^{T2} is NH₂.
(6) In one embodiment, X^{T} is N and R^{T1} is (CH₂)₀₋₃C(O)NH. In one embodiment, X^{T} is CH and R^{T1} is (CH₂)C(O)NH.
(7) In one embodiment, X^{T} is N and R^{T1} is absent.
(8) In one embodiment, X^{T} is N and R^{T1} is (CH₂)₀₋₃NHC(O). In one embodiment, X^{T} is CH and R^{T1} is (CH₂)NHC(O).
(9) In one embodiment, X^{T} is N and R^{T2} is NO₂.
(10) In one embodiment, X^{T} is N; R^{T2} is NH₂.
(11) In one embodiment, R^{T1} is (CH₂)₀₋₃C(O)NH and R^{T2} is NO₂. In a further embodiment, R^{T1} is (CH₂)C(O)NH.
(12) In one embodiment, R^{T1} is (CH₂)₀₋₃C(O)NH; R^{T2} is NH₂.
(13) In one embodiment, R^{T1} is (CH₂)C(O)NH.; R^{T2} is NH₂.
(14) In one embodiment, R^{T1} is absent and R^{T2} is NO₂.
(15) In one embodiment, R^{T1} is absent; R^{T2} is NH₂.
(16) In one embodiment, R^{T1} is (CH₂)₀₋₃NHC(O) and R^{T2} is NO₂. In a further embodiment, R^{T1} is (CH₂)NHC(O).
(17) In one embodiment, R^{T1} is (CH₂)₀₋₃NHC(O); R^{T2} is NH₂.
(18) In one embodiment, R^{T1} is (CH₂)NHC(O); R^{T2} is NH₂.
(19) In one embodiment, X^{T} is CH; R^{T1} and R^{T2} are each as defined in any of (11)-(18). In a further embodiment, R^{T1} and R^{T2} are each as defined in any of (11)-(13). In another further embodiment, R^{T1} and R^{T2} are each as defined in any of (14)-(15). In another further embodiment, R^{T1} and R^{T2} are each as defined in any of (16)-(18).
(20) In one embodiment, X^{T} is N; R^{T1} and R^{T2} are each as defined in any of (11)-(18). In a further embodiment, R^{T1} and R^{T2} are each as defined in any of (11)-(13). In another further embodiment, R^{T1} and R^{T2} are each as defined in any of (14)-(15). In another further embodiment, R^{T1} and R^{T2} are each as defined in any of (16)-(18).
(21) In one embodiment, R^{T7} is H; and X^{T}, R^{T1} and R^{T2} are each as defined in any of (1)-(20).
(22) In one embodiment, R^{TN1} and R^{TN2} are each H; and X^{T}, R^{T1} and R^{T2} are each as defined in any of (1)-(20).
(23) In one embodiment, R^{T7} is H; R^{TN1} and R^{TN2} are each H; and X^{T}, R^{T1} and R^{T2} are each as defined in any of (1)-(20).
(24) In one embodiment, Tn1 is 0, 1, or 2; and X^{T}, R^{T1} and R^{T2} are each as defined in any of (1)-(20). In a further embodiment, Tn1 is 0.
(25) In one embodiment, Tn1 is 1 or 2; and X^{T}, R^{T1}, R^{T1} and R^{T2} are each as defined in any of (1)-(20). In a further embodiment, at least one R^{T5} is OH, halogen, or CN. In a further embodiment, at least one R^{T5} is halogen. In a further embodiment, at least one R^{T5} is F or Cl. In another further embodiment, at least one R^{T5} is C₁-C₄ alkyl, including but not limited to methyl, ethyl, n-propyl, i-propyl, n-butyl, i-butyl, or t-butyl or C₁-C₄ alkyl, including but not limited to methyl, ethyl, n-propyl, i-propyl, n-butyl, i-butyl, or t-butyl substituted with halogen. In a further embodiment, at least one R^{T5} is C₁-C₄ alkyl, including but not limited to methyl, ethyl, n-propyl, i-propyl, n-butyl, i-butyl, or t-butyl. In another further embodiment, at least one R^{T5} is C₁-C₄ alkoxy, including but not limited to methoxy, ethoxy, n-propoxy, i-propoxy, n-butoxy, i-butoxy, or t-butoxy or C₁-C₄ alkoxy, including but not limited to methoxy, ethoxy, n-propoxy, i-propoxy, n-butoxy, i-butoxy, or t-butoxy substituted with halogen.
(26) In one embodiment, Tn2 is 0 or 1; and X^{T}, R^{T1} and R^{T2} are each as defined in any of (1)-(20). In a further embodiment, Tn2 is 0.
(27) In one embodiment, Tn2 is 1; and X^{T}, R^{T1}, R^{T1} and R^{T2} are each as defined in any of (1)-(20). In a further embodiment, at least one R^{T6} is OH, halogen, or CN. In a further embodiment, at least one R^{T6} is halogen. In a further embodiment, at least one R^{T6} is F or C₁. In another further embodiment, at least one R^{T6} is C₁-C₄ alkyl, including but not limited to methyl, ethyl, n-propyl, i-propyl, n-butyl, i-butyl, or t-butyl or C₁-C₄ alkyl, including but not limited to methyl, ethyl, n-propyl, i-propyl, n-butyl, i-butyl, or t-butyl substituted with halogen. In another further embodiment, at least one R^{T6} is C₁-C₄ alkoxy, including but not limited to methoxy, ethoxy, n-propoxy, i-propoxy, n-butoxy, i-butoxy, or t-butoxy or C₁-C₄ alkoxy, including but not limited to methoxy, ethoxy, n-propoxy, i-propoxy, n-butoxy, i-butoxy, or t-butoxy substituted with halogen.
(28) In one embodiment, R^{T7} is H; and Tn1, X^{T}, R^{T1}, R^{T2}, and R^{T5} are each as defined in any of (24)-(25).
(29) In one embodiment, R^{TN1} and R^{TN2} are each H; and Tn1, X^{T}, R^{T1}, R^{T2}, and R^{T5} are each as defined in any of (24)-(25).
(30) In one embodiment, R^{T7} is H; R^{TN1} and R^{TN2} are each H; and Tn1, X^{T}, R^{T1}, R^{T2}, and R^{T5} are each as defined in any of (24)-(25).
(31) In one embodiment, R^{T7} is H; and Tn2, X^{T}, R^{T1}, R^{T2}, and R^{T6} are each as defined in any of (26)-(27).
(32) In one embodiment, R^{TN1} and R^{TN2} are each H; and Tn2, X^{T}, R^{T1}, R^{T2}, and R^{T6} are each as defined in any of (26)-(27).
(33) In one embodiment, R^{T7} is H; R^{TN1} and R^{TN2} are each H; and Tn2, X^{T}, R^{T1}, R^{T2}, and R^{T6} are each as defined in any of (26)-(27).
(34) In one embodiment, R^{T7} is H; and Tn1, Tn2, X^{T}, R^{T1}, R^{T2}, R^{T5}, and R^{T6} are each as defined in any of (24)-(27).
(35) In one embodiment, R^{TN1} and R^{TN2} are each H; and Tn1, Tn2, X^{T}, R^{T1}, R^{T2}, R^{T5}, and R^{T6} are each as defined in any of (24)-(27).
(36) In one embodiment, R^{T7} is H; R^{TN1} and R^{TN2} are each H; and Tn1, Tn2, X^{T}, R^{T1}, R^{T2}, R^{T5}, and R^{T6} are each as defined in any of (24)-(27).

In one embodiment, L is any of the groups described herein; and XT, Tn1, Tn2, R^{T1}, R^{T2}, R^{T5}, R^{T6}, R^{T7}, R^{TN1}, and R^{TN2} are each independently selected from any of the groups selected from (1)-(36) described herein.

In one embodiment, the compound of Formula TL-I is of Formula TL-Ia or TL-Ib:

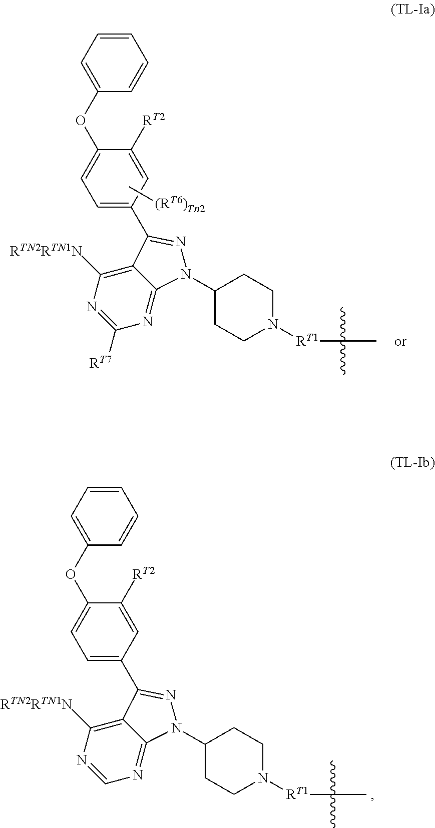

wherein R^{T1}, R^{T2}, R^{T6}, R^{T7}, R^{TN1}, R^{TN2}, Tn2 are each as defined above in Formula TL-I.

In one embodiment, R^{T2} is NO₂.

In one embodiment, R^{T2} is NH₂.

R^{T1}, R^{T6}, R^{T7}, R^{TN1}, R^{TN2}, and Tn2 can each be selected from any of the groups and combined as described above in Formula TL-I, and may further be combined with any of the groups described for R^{T2} herein.

In one embodiment, L is any of the groups described herein; and Tn2, R^{T1}, R^{T2}, R^{T6}, R^{T7}, R^{TN1}, and R^{TN2} are each independently selected from any of the groups and combined as described herein.

### Degron

The Degron serves to link a targeted protein, through a Linker and a Targeting Ligand, to a ubiquitin ligase for proteosomal degradation. In one embodiment, the Degron is capable of binding to a ubiquitin ligase, such as an E3 ubiquitin ligase. In one embodiment, the Degron is capable of binding to cereblon. In one embodiment, the E3 ubiquitin ligase is the Cul4-Rbx1-DDB 1-cereblon complex. In one embodiment, the E3 ubiquitin-ligase is MDM2 (mouse double minute 2 homolog). In one embodiment, the E3 ubiquitin-ligase is CHIP (C terminus of HSC70-Interacting Protein). In one embodiment, the E3 ubiquitin-ligase is MARCH1 (Membrane-associated RING-CH protein I). In one embodiment, the E3 ubiquitin-ligase is Parkin. In one embodiment the E3 ubiquitin-ligase is Rictor. In one embodiment, the E3 ubiquitin-ligase is SMURF1 (SMAD specific E3 ubiquitin protein ligase 1). In one embodiment, the E3 ubiquitin-ligase is SMURF2 (SMAD specific E3 ubiquitin protein ligase 2). In one embodiment, the E3 ubiquitin-ligase is UBR1 (Ubiquitin Protein Ligase E3 Component N-Recognin 1). In one embodiment, the E3 ubiquitin-ligase is UBR2 (Ubiquitin Protein Ligase E3 Component N-Recognin 2). In one embodiment, the E3 ubiquitin-ligase is TRIM63 (Tripartite motif containing 63). In one embodiment, the E3 ubiquitin-ligase is VHL (Von Hippel-Lindau disease tumor suppressor). Compounds that bind to these ligases are known in the literature and thus are available to one of ordinary skill in the art.

In one embodiment, the Degron is of Formula D1:

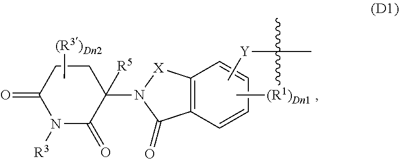

or an enantiomer, diastereomer, or stereoisomer thereof, wherein:

Y is a bond, (CH₂)₁₋₆, (CH₂)₀₋₆—O, (CH₂)₀₋₆—C(O)NR^{2′}, (CH₂)₀₋₆—NR^{2′}C(O), (CH₂)₀₋₆—NH, or (CH₂)₀₋₆—NR²;
X is C(O) or C(R³)₂;
each R¹ is independently halogen, OH, C₁-C₆ alkyl, or C₁-C₆ alkoxy;
R² is C₁-C₆ alkyl or C(O)—C₁-C₆ alkyl;
R^{2′} is H or C₁-C₆ alkyl;
each R³ is independently H or C₁-C₃ alkyl;
each R^{3′} is independently C₁-C₃ alkyl;
R⁵ is H, deuterium, C₁-C₃ alkyl, F, or Cl;
Dn1 is 0, 1, 2 or 3; and
Dn2 is 0, 1 or 2,
   wherein the Degron is covalently bonded to another moiety via

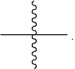

In one embodiment the Degron is covalently bonded to another compound. In a further embodiment the Degron is covalently bonded to a Linker.

In one embodiment, X is C(O).

In one embodiment, X is C(R³)₂; and each R³ is H. In one embodiment, X is C(R³)₂, and one of R³ is H, and the other is C₁-C₃ alkyl selected from methyl, ethyl, and propyl. In one embodiment, X is C(R³)₂; and each R³ is independently selected from methyl, ethyl, and propyl.

In one embodiment, Y is a bond.

In one embodiment, Y is (CH₂)₁, (CH₂)₂, (CH₂)₃, (CH₂)₄, (CH₂)₅, or (CH₂)₆. In one embodiment, Y is (CH₂)₁, (CH₂)₂, or (CH₂)₃. In one embodiment, Y is (CH₂)₁ or (CH₂)₂.

In one embodiment, Y is O, CH₂—O, (CH₂)₂₋₀, (CH₂)₃₋₀, (CH₂)₄₋₀, (CH₂)₅₋₀, or (CH₂)₆—O. In one embodiment, Y is O, CH₂—O, (CH₂)₂₋₀, or (CH₂)₃₋₀. In one embodiment, Y is O or CH₂—O. In one embodiment, Y is O.

In one embodiment, Y is C(O)NR^{2′}, CH₂—C(O)NR^{2′}, (CH₂)₂—C(O)NR^{2′}, (CH₂)₃—C(O)NR^{2′}, (CH₂)₄—C(O)NR^{2′}, (CH₂)₅—C(O)NR^{2′}, or (CH₂)₆—C(O)NR^{2′}. In one embodiment, Y is C(O)NR^{2′}, CH₂—C(O)NR^{2′}, (CH₂)₂—C(O)NR^{2′}, or (CH₂)₃—C(O)NR^{2′}. In one embodiment, Y is C(O)NR^{2′} or CH₂—C(O)NR^{2′}. In one embodiment, Y is C(O)NR^{2′}.

In one embodiment, Y is NR^{2′}C(O), CH₂—NR^{2′}C(O), (CH₂)₂—NR^{2′}C(O), (CH₂)₃—NR^{2′}C(O), (CH₂)₄—NR^{2′}C(O), (CH₂)₅—NR^{2′}C(O), or (CH₂)₆—NR^{2′}C(O). In one embodiment, Y is NR^{2′}C(O), CH₂—NR^{2′}C(O), (CH₂)₂—NR^{2′}C(O), or (CH₂)₃—NR^{2′}C(O). In one embodiment, Y is NR^{2′}C(O) or CH₂—NR^{2′}C(O). In one embodiment, Y is NR^{2′}C(O).

In one embodiment, R^{2′} is H. In one embodiment, R^{2′} is selected from methyl, ethyl, propyl, butyl, i-butyl, t-butyl, pentyl, i-pentyl, and hexyl. In one embodiment, R^{2′} is C₁-C₃ alkyl selected from methyl, ethyl, and propyl.

In one embodiment, Y is NH, CH₂—NH, (CH₂)₂—NH, (CH₂)₃—NH, (CH₂)₄—NH, (CH₂)₅—NH, or (CH₂)₆—NH. In one embodiment, Y is NH, CH₂—NH, (CH₂)₂—NH, or (CH₂)₃—NH. In one embodiment, Y is NH or CH₂—NH. In one embodiment, Y is NH.

In one embodiment, Y is NR², CH₂—NR², (CH₂)₂—NR², (CH₂)₃—NR², (CH₂)₄—NR², (CH₂)₅—NR², or (CH₂)₆—NR². In one embodiment, Y is NR², CH₂—NR², (CH₂)₂—NR², or (CH₂)₃—NR². In one embodiment, Y is NR² or CH₂—NR². In one embodiment, Y is NR².

In one embodiment, R² is selected from methyl, ethyl, propyl, butyl, i-butyl, t-butyl, pentyl, i-pentyl, and hexyl. In one embodiment, R² is C₁-C₃ alkyl selected from methyl, ethyl, and propyl.

In one embodiment, R² is selected from C(O)-methyl, C(O)-ethyl, C(O)-propyl, C(O)-butyl, C(O)-i-butyl, C(O)-t-butyl, C(O)-pentyl, C(O)-i-pentyl, and C(O)-hexyl. In one embodiment, R² is C(O)—C₁-C₃ alkyl selected from C(O)-methyl, C(O)-ethyl, and C(O)-propyl.

In one embodiment, R³ is H.

In one embodiment, R³ is C₁-C₃ alkyl selected from methyl, ethyl, and propyl. In one embodiment, R³ is methyl.

In one embodiment, Dn2 is 0.

In one embodiment, Dn2 is 1.

In one embodiment, Dn2 is 2.

In one embodiment, each R^{3′} is independently C₁-C₃ alkyl selected from methyl, ethyl, and propyl.

In one embodiment, Dn1 is 0.

In one embodiment, Dn1 is 1.

In one embodiment, Dn1 is 2.

In one embodiment, Dn1 is 3.

In one embodiment, each R¹ is independently selected from halogen, OH, C₁-C₆ alkyl, including but not limited to methyl, ethyl, propyl, butyl, i-butyl, t-butyl, pentyl, i-pentyl, and hexyl, and C₁-C₆ alkoxy, including but not limited to methoxy, ethoxy, propoxy, butoxy, i-butoxy, t-butoxy, and pentoxy. In a further embodiment, each R¹ is independently selected from F, C₁, OH, methyl, ethyl, propyl, butyl, i-butyl, t-butyl, methoxy, and ethoxy.

In one embodiment, R⁵ is H, deuterium, or C₁-C₃ alkyl. In a further embodiment, R⁵ is in the (S) or (R) configuration. In a further embodiment, R⁵ is in the (S) configuration. In one embodiment, the compound comprises a racemic mixture of (S)—R⁵ and (R)—R⁵.

In one embodiment, R⁵ is H.

In one embodiment, R⁵ is deuterium.

In one embodiment, R⁵ is C₁-C₃ alkyl selected from methyl, ethyl, and propyl. In one embodiment, R⁵ is methyl.

In one embodiment, R⁵ is F or C₁. In a further embodiment, R⁵ is in the (S) or (R) configuration. In a further embodiment, R⁵ is in the (R) configuration. In one embodiment, the compound comprises a racemic mixture of (S)—R⁵ and (R)—R⁵. In one embodiment, R⁵ is F.

Any of the groups described herein for any of X, Y, Dn1, Dn2, R¹, R², R^{2′}, R³, R^{3′}, and R⁵ can be combined with any of the groups described herein for one or more of the remainder of X, Y, Dn1, Dn2, R¹, R², R^{2′}, R³, R^{3′}, and R⁵, and may further be combined with any of the groups described herein for the Linker.
(1) In one embodiment, X is C(O) and Y is a bond.
(2) In one embodiment, X is C(O) and Y is (CH₂)₀₋₆—O. In a further embodiment, Y is O.
(3) In one embodiment, X is C(O); Y is a bond; and Dn1 and Dn2 are each 0.
(4) In one embodiment, X is C(O); Y is a bond; and R³ is H.
(5) In one embodiment, X is C(O); Y is a bond; and R⁵ is H.
(6) In one embodiment, X is C(O); Y is a bond; and R³ is H; and R⁵ is H.
(7) In one embodiment, X is C(O); Y is (CH₂)₀₋₆—O; and R³ is H. In a further embodiment, Y is O.
(8) In one embodiment, X is C(O); Y is (CH₂)₀₋₆—O; and R⁵ is H. In a further embodiment, Y is O.
(9) In one embodiment, X is C(O); Y is (CH₂)₀₋₆—O; R³ is H; and R⁵ is H. In a further embodiment, Y is O.
(10) In one embodiment, Dn1 and Dn2 are each 0; and X, Y, R¹, R³, and R⁵ are each as defined in any of (1)-(9).

In one embodiment, the Degron is of Formula D1a or D b:

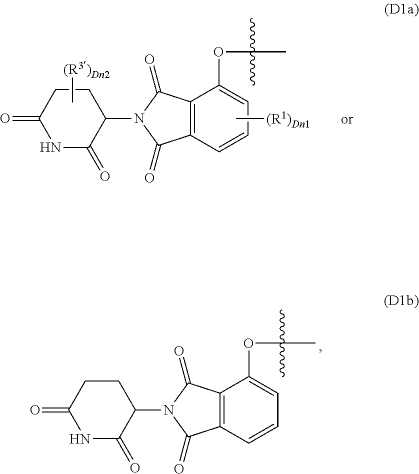

or an enantiomer, diastereomer, or stereoisomer thereof, wherein R¹, R^{3′}, Dn1, and Dn2 are each as defined above in Formula D1, and can be selected from any moieties or combinations thereof described above.

In one embodiment, the Degron is of Formula D2:

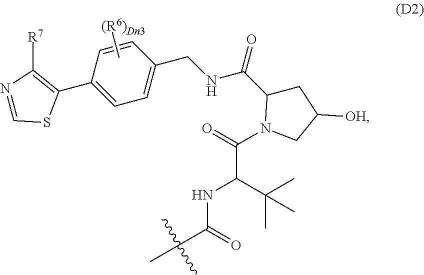

or an enantiomer, diastereomer, or stereoisomer thereof, wherein:
each R⁶ is independently C₁-C₃ alkyl;
Dn3 is 0, 1, 2, 3 or 4; and R⁷ is C₁-C₃ alkyl,
   wherein the Degron is covalently bonded to another moiety via

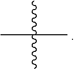

In one embodiment the Degron is covalently bonded to another compound. In a further embodiment the Degron is covalently bonded to a Linker.

In one embodiment, Dn3 is 0.

In one embodiment, Dn3 is 1.

In one embodiment, Dn3 is 2.

In one embodiment, Dn3 is 3.

In one embodiment, each R⁶ is independently C₁-C₃ alkyl selected from methyl, ethyl, and propyl.

In one embodiment, R⁷ is methyl, ethyl, or propyl. In one embodiment, R⁷ is methyl.

In one embodiment, the Degron is of Formula D2a or D2b:

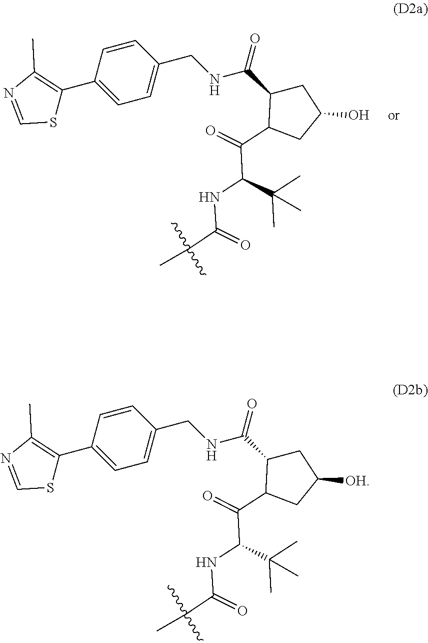 [SMILES: *C(=O)N[C@@H](C(=O)C1C[C@@H](O)C[C@@H]1C(=O)NCc1ccc(-c2scnc2C)cc1)C(C)(C)C ; *C(=O)N[C@H](C(=O)C1C[C@H](O)C[C@H]1C(=O)NCc1ccc(-c2scnc2C)cc1)C(C)(C)C]

### Linker

The Linker is a bond or a carbon chain that serves to link a Targeting Ligand with a Degron.

In one embodiment, the carbon chain optionally comprises one, two, three, or more heteroatoms selected from N, O, and S. In one embodiment, the carbon chain comprises only saturated chain carbon atoms. In one embodiment, the carbon chain optionally comprises two or more unsaturated chain carbon atoms, such as C═C or C≡C. In one embodiment, one or more chain carbon atoms in the carbon chain are optionally substituted with one or more substituents, including but not limited to oxo, C₁-C₆ alkyl, C₂-C₆ alkenyl, C₂-C₆ alkynyl, C₁-C₃ alkoxy, OH, halogen, NH₂, NH(C₁-C₃ alkyl), N(C₁-C₃ alkyl)₂, CN, C₃—C cycloalkyl, heterocyclyl, phenyl, and heteroaryl.

In one embodiment, the Linker comprises at least 5 chain atoms selected from C, O, N, and S atoms. In one embodiment, the Linker comprises less than 20 chain atoms selected from C, O, N, and S atoms. In one embodiment, the Linker comprises 5, 6, 7, 8, 9, 10, 11, 12, 13, 14, 15, 16, 17, 18, or 19 chain atoms selected from C, O, N, and S atoms. In one embodiment, the Linker comprises 5, 7, 9, 11, 13, 15, 17, or 19 chain atoms selected from C, O, N, and S atoms. In one embodiment, the Linker comprises 5, 7, 9, or 11 chain atoms selected from C, O, N, and S atoms. In one embodiment, the Linker comprises 6, 8, 10, 12, 14, 16, or 18 chain atoms selected from C, O, N, and S atoms. In one embodiment, the Linker comprises 6, 8, 10, or 12 chain atoms selected from C, O, N, and S.

In one embodiment, the Linker comprises from 1 to 5 chain atoms selected from C, O, N, and S atoms.

In one embodiment, the Linker is a carbon chain optionally substituted with non-bulky substituents, including but not limited to oxo, C₁-C₆ alkyl, C₂-C₆ alkenyl, C₂-C₆ alkynyl, C₁-C₃ alkoxy, OH, halogen, NH₂, NH(C₁-C₃ alkyl), N(C₁-C₃ alkyl)₂, and CN. In one embodiment, the non-bulky substitution is located on the chain carbon atom proximal to the Degron. In one embodiment, the carbon atom substituted with the non-bulky substituent is separated from the carbon atom to which the Degron is bonded by at least 3, 4, or 5 chain atoms in the Linker.

In one embodiment, the Linker is of Formula L0:

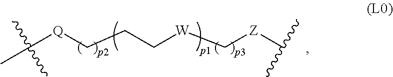

or an enantiomer, diastereomer, or stereoisomer thereof, wherein

p1 is an integer selected from 0 to 12;

p2 is an integer selected from 0 to 12;

p3 is an integer selected from 1 to 6;

each W is independently absent, CH₂, O, S, NH, or NR⁸;

Z is absent, CH₂, O, NH, or NR⁸;

each R⁸ is independently C₁-C₃ alkyl; and

Q is absent or CH₂C(O)NH,

wherein the Linker is covalently bonded to a Degron via the

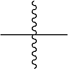

next to Q, and covalently bonded to a Targeting Ligand via the

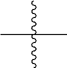

next to Z.

In one embodiment, the total number of chain atoms in the Linker is less than 30. In a further embodiment, the total number of chain atoms in the Linker is less than 20.

In one embodiment, p1 is an integer selected from 0 to 10.

In one embodiment, p1 is an integer selected from 1 to 10.

In one embodiment, p1 is selected from 1, 2, 3, 4, 5, and 6.

In one embodiment, p1 is 0, 1, 3, or 5.

In one embodiment, p1 is 0, 1, 2, or 3.

In one embodiment, p1 is 0.

In one embodiment, p1 is 3.

In one embodiment, p2 is an integer selected from 0 to 10.

In one embodiment, p2 is selected from 0, 1, 2, 3, 4, 5, and 6.

In one embodiment, p2 is 0, 1, 2, or 3.

In one embodiment, p2 is 0.

In one embodiment, p2 is 1.

In one embodiment, p3 is an integer selected from 1 to 5.

In one embodiment, p3 is 2, 3, 4, or 5.

In one embodiment, p3 is 0, 1, 2, or 3.

In one embodiment, p3 is 0.

In one embodiment, p3 is 2 or 3.

In one embodiment, at least one W is CH₂.

In one embodiment, at least one W is O.

In one embodiment, at least one W is S.

In one embodiment, at least one W is NH.

In one embodiment, at least one W is NR⁸; and R⁸ is C₁-C₃ alkyl selected from methyl, ethyl, and propyl.

In one embodiment, each W is O.

In one embodiment, Z is absent.

In one embodiment, Z is CH₂.

In one embodiment, Z is O.

In one embodiment, Z is NH.

In one embodiment, Z is NR⁸; and R⁸ is C₁-C₃ alkyl selected from methyl, ethyl, and propyl.

In one embodiment, Z is part of the Targeting Ligand that is bonded to the Linker, namely, Z is formed from reacting a functional group of the Targeting Ligand with the Linker.

In one embodiment, Q is absent.

In one embodiment, the Linker-Targeting Ligand has the structure selected from:

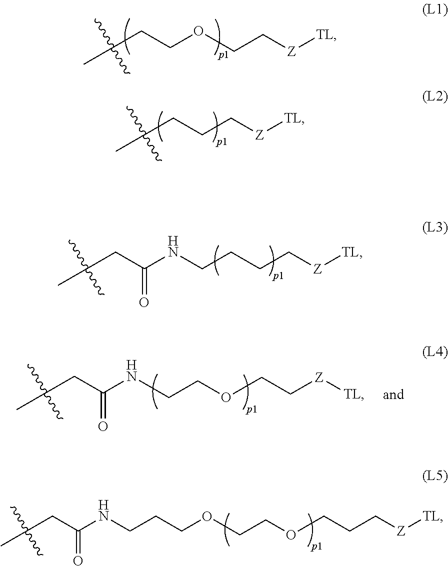

wherein Z, TL, and p1 are each as described above.

In one embodiment, p1 is 0, 1, 2, or 3. In one embodiment, p1 is 0. In one embodiment, p1 is 2. In one embodiment, p1 is 1. In one embodiment, p1 is 3.

In one embodiment, Z is absent. In one embodiment, Z is CH₂.

In one embodiment, p1 is 0 and Z is absent.

In one embodiment, p1 is 1 and Z is absent.

In one embodiment, p1 is 2 and Z is absent.

In one embodiment, p1 is 3 and Z is absent.

Any one of the Degrons described herein can be covalently bound to any one of the Linkers described herein. Any one of the Targeting Ligands described herein can be covalently bound to any one of the Linkers described herein.

In one embodiment, the invention provides the Degron-Linker (DL), wherein the Degron is of Formula D1, and the Linker is selected from L1-L5. In one embodiment, the Degron is of Formula D1a or D1b, and the Linker is selected from L1-L5. In one embodiment, the Degron is of Formula D1a or D1b, and the Linker is L3, L4, or L5. In one embodiment, the Degron is of Formula D1b, and the Linker is L3, L4, or L5.

In one embodiment, the invention provides the Degron-Linker (DL), wherein the Degron is of Formula D2, and the Linker is selected from L1-L5. In one embodiment, the Degron is of Formula D2a or D2b, and the Linker is selected from L1-L5. In one embodiment, the Degron is of Formula D2a or D2b, and the Linker is L1 or L2.

In one embodiment, the Linker is designed and optimized based on SAR (structure-activity relationship) and X-ray crystallography of the Targeting Ligand with regard to the location of attachment for the Linker.

In one embodiment, the optimal Linker length and composition vary by the Targeting Ligand and can be estimated based upon X-ray structure of the Targeting Ligand bound to its target. Linker length and composition can be also modified to modulate metabolic stability and pharmacokinetic (PK) and pharmacodynamics (PD) parameters.

In one embodiment, the invention provides a compound selected from Formula II:

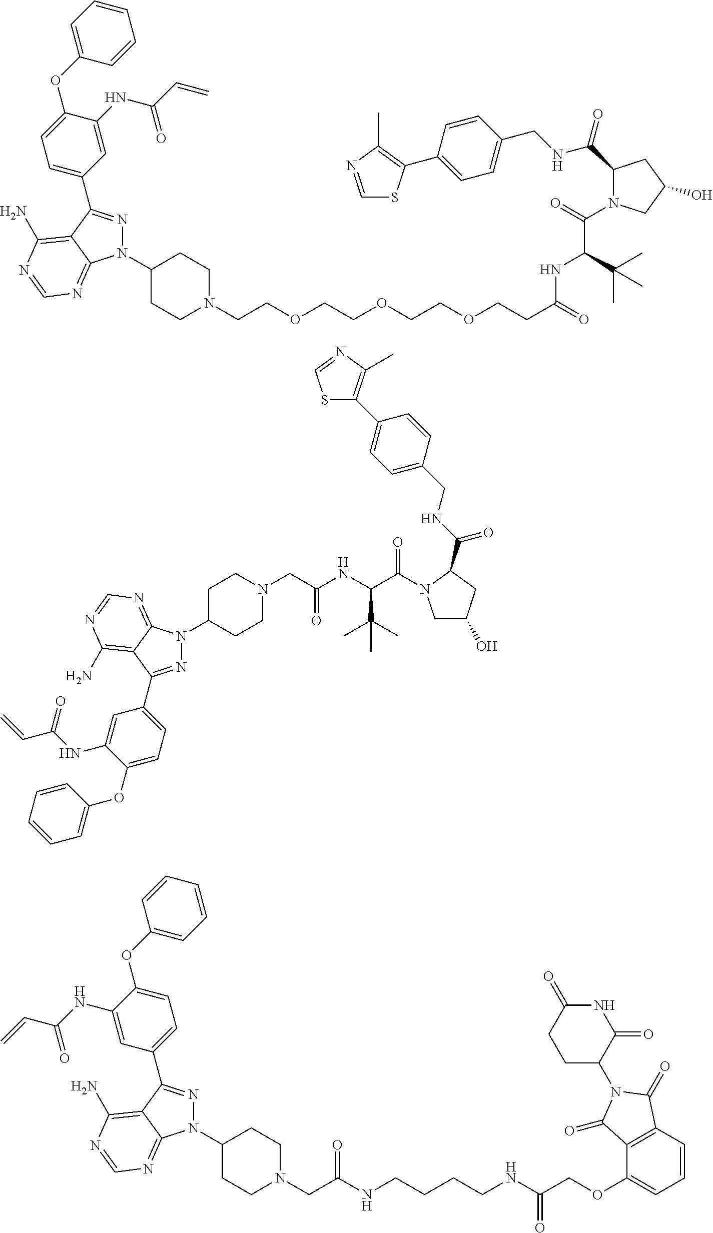

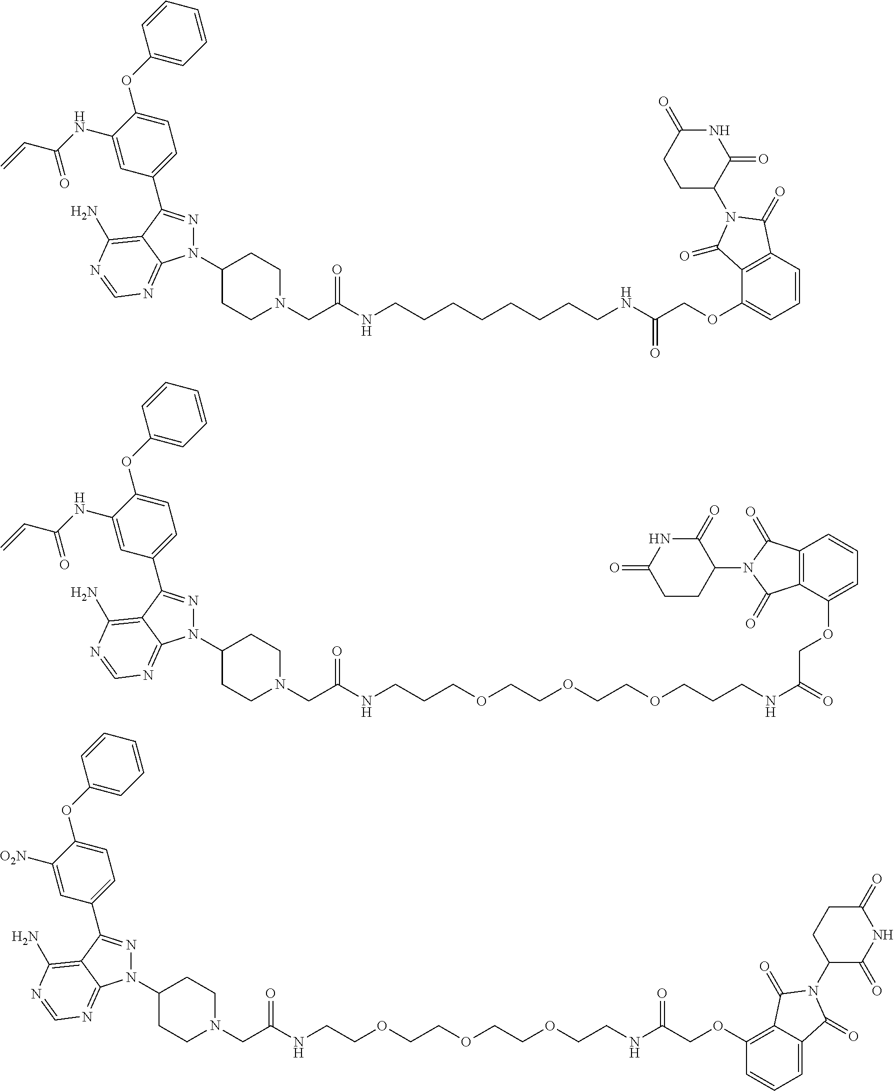 [SMILES: C=CC(=O)Nc1cc(-c2nn(C3CCN(CC(=O)NCCCCCCCCNC(=O)COc4cccc5c4C(=O)N(C4CCC(=O)NC4=O)C5=O)CC3)c3ncnc(N)c23)ccc1Oc1ccccc1 ; C=CC(=O)Nc1cc(-c2nn(C3CCN(CC(=O)NCCCOCCOCCOCCCNC(=O)COc4cccc5c4C(=O)N(C4CCC(=O)NC4=O)C5=O)CC3)c3ncnc(N)c23)ccc1Oc1ccccc1 ; Nc1ncnc2c1c(-c1ccc(Oc3ccccc3)c([N+](=O)[O-])c1)nn2C1CCN(CC(=O)NCCOCCOCCOCCNC(=O)COc2cccc3c2C(=O)N(C2CCC(=O)NC2=O)C3=O)CC1]

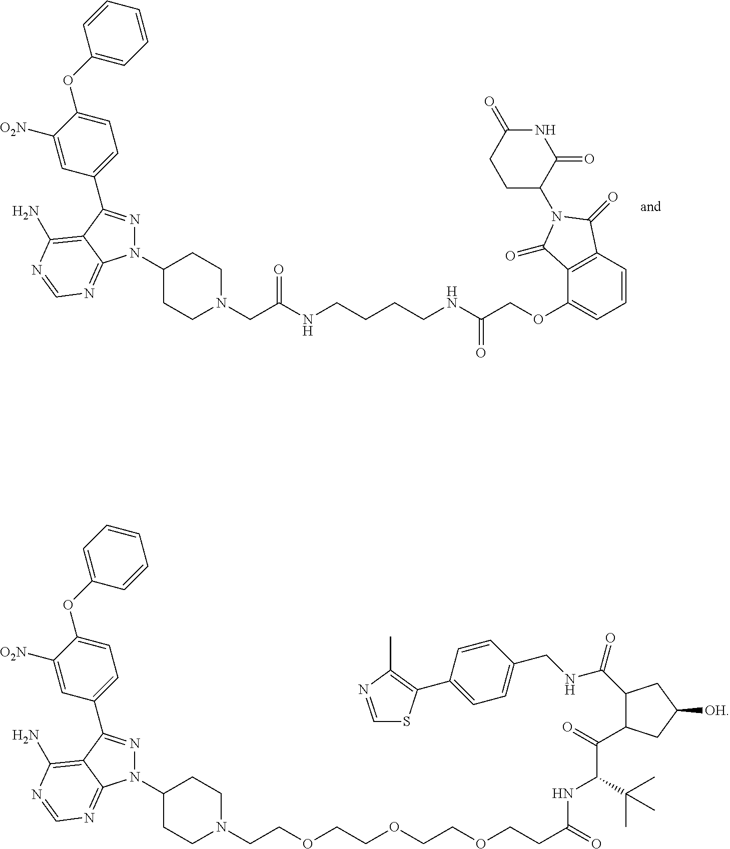 [SMILES: Nc1ncnc2c1c(-c1ccc(Oc3ccccc3)c([N+](=O)[O-])c1)nn2C1CCN(CC(=O)NCCCCNC(=O)COc2cccc3c2C(=O)N(C2CCC(=O)NC2=O)C3=O)CC1 ; Cc1ncsc1-c1ccc(CNC(=O)C2C[C@@H](O)CC2C(=O)[C@@H](NC(=O)CCOCCOCCOCCN2CCC(n3nc(-c4ccc(Oc5ccccc5)c([N+](=O)[O-])c4)c4c(N)ncnc43)CC2)C(C)(C)C)cc1]

Some embodiments of invention include the bifunctional compounds having the following structures, their synthesis and methods of use:

| Cmpd No. | Structure |
|---|---|
| PP1 | 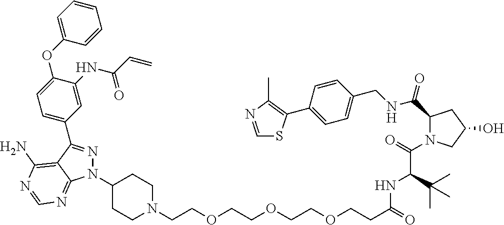 [SMILES: C=CC(=O)Nc1cc(-c2nn(C3CCN(CCOCCOCCOCCC(=O)N[C@@H](C(=O)N4C[C@@H](O)C[C@@H]4C(=O)NCc4ccc(-c5scnc5C)cc4)C(C)(C)C)CC3)c3ncnc(N)c23)ccc1Oc1ccccc1] |
| PP2 | 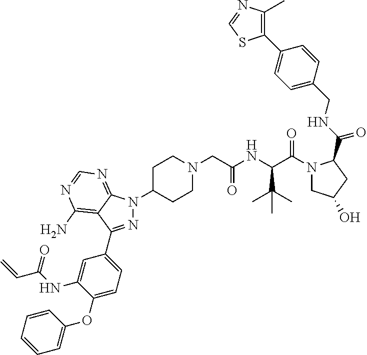 [SMILES: C=CC(=O)Nc1cc(-c2nn(C3CCN(CC(=O)N[C@@H](C(=O)N4C[C@@H](O)C[C@@H]4C(=O)NCc4ccc(-c5scnc5C)cc4)C(C)(C)C)CC3)c3ncnc(N)c23)ccc1Oc1ccccc1] |
| PP3 | 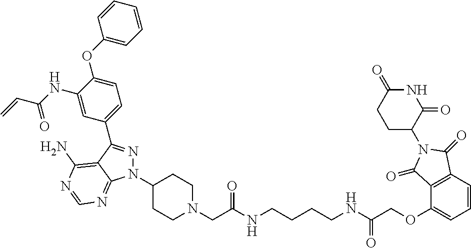 [SMILES: C=CC(=O)Nc1cc(-c2nn(C3CCN(CC(=O)NCCCCNC(=O)COc4cccc5c4C(=O)N(C4CCC(=O)NC4=O)C5=O)CC3)c3ncnc(N)c23)ccc1Oc1ccccc1] |
| PP4 | 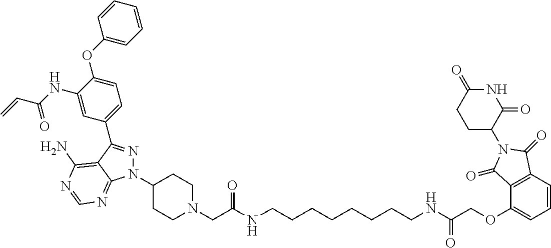 [SMILES: C=CC(=O)Nc1cc(-c2nn(C3CCN(CC(=O)NCCCCCCCCNC(=O)COc4cccc5c4C(=O)N(C4CCC(=O)NC4=O)C5=O)CC3)c3ncnc(N)c23)ccc1Oc1ccccc1] |
| PP5 | 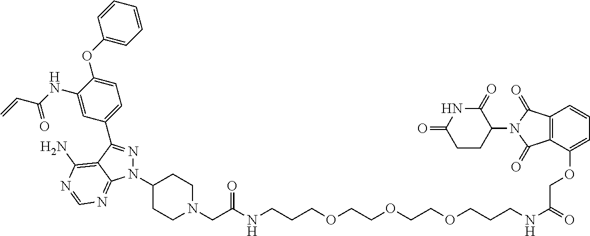 [SMILES: C=CC(=O)Nc1cc(-c2nn(C3CCN(CC(=O)NCCCOCCOCCOCCCNC(=O)COc4cccc5c4C(=O)N(C4CCC(=O)NC4=O)C5=O)CC3)c3ncnc(N)c23)ccc1Oc1ccccc1] |
| PP6 | 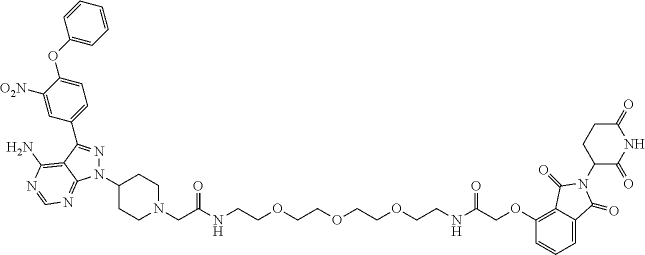 [SMILES: Nc1ncnc2c1c(-c1ccc(Oc3ccccc3)c([N+](=O)[O-])c1)nn2C1CCN(CC(=O)NCCOCCOCCOCCNC(=O)COc2cccc3c2C(=O)N(C2CCC(=O)NC2=O)C3=O)CC1] |
| PP7 | 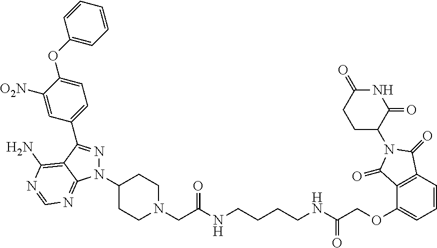 [SMILES: Nc1ncnc2c1c(-c1ccc(Oc3ccccc3)c([N+](=O)[O-])c1)nn2C1CCN(CC(=O)NCCCCNC(=O)COc2cccc3c2C(=O)N(C2CCC(=O)NC2=O)C3=O)CC1] |
| PP8 | 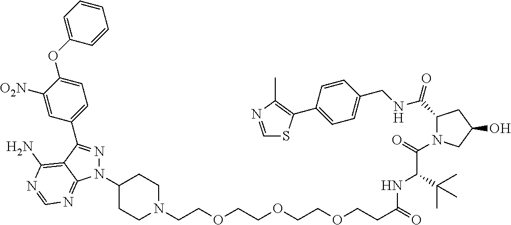 [SMILES: Cc1ncsc1-c1ccc(CNC(=O)[C@@H]2C[C@@H](O)CN2C(=O)[C@@H](NC(=O)CCOCCOCCOCCN2CCC(n3nc(-c4ccc(Oc5ccccc5)c([N+](=O)[O-])c4)c4c(N)ncnc43)CC2)C(C)(C)C)cc1] |

Some of the foregoing compounds can comprise one or more asymmetric centers, and thus can exist in various isomeric forms. In one embodiment the compounds exist as stereoisomers. In a further embodiment the compounds exist as diastereomers. Accordingly, compounds of the application may be in the form of an individual enantiomer, diastereomer or geometric isomer, or may be in the form of a mixture of stereoisomers. In one embodiment, the compounds of the application are enantiopure compounds. In another embodiment, mixtures of stereoisomers or diastereomers are provided.

Furthermore, certain compounds, as described herein, may have one or more double bonds that can exist as either the Z or E isomer, unless otherwise indicated. The application additionally encompasses the compounds as individual Z/E isomers substantially free of other E/Z isomers and alternatively, as mixtures of various isomers.

In one embodiment, the invention provides compounds that target proteins, such as a HER family protein, for degradation. In a further embodiment, the HER family protein is Her3. These compounds have numerous advantages, such as kinase activity, over inhibitors of protein function, and can a) overcome resistance in certain cases; b) prolong the kinetics of drug effect by destroying the protein, thus requiring resynthesis of the protein even after the compound has been metabolized; c) target all functions of a protein at once rather than a specific catalytic activity or binding event; d) expand the number of drug targets by including all proteins that a ligand can be developed for, rather than proteins whose activity, such as kinase activity, can be affected by a small molecule inhibitor, antagonist or agonist; and e) have increased potency compared to inhibitors due to the possibility of the small molecule acting catalytically.

Some embodiments of the invention relate to degradation or loss of 30% to 100% of the target protein. Some embodiments relate to the loss of 50-100% of the target protein. Other embodiments relate to the loss of 75-95% of the targeted protein.

A bifunctional compound of any of the formulae described herein, or selected from any bifunctional compounds described herein of the invention is capable of modulating or decreasing the amount of a targeted protein. In one embodiment the targeted protein is a HER family protein. In a further embodiment, the HER family protein is Her3.

A bifunctional compound of any of the formulae described herein, or selected from any bifunctional compounds described herein of the invention is also capable of degrading a targeted protein through the UPP pathway. In one embodiment the targeted protein is a HER family protein. In a further embodiment, the HER family protein is Her3.

A bifunctional compound of any of the formulae described herein, or selected from any bifunctional compounds described herein of the invention is also capable of preventing dimer formation between HER family member proteins, such as dimer formation between EGFR, Her2, or Her4 and Her3. Accordingly, a bifunctional compound of any of the formulae described herein, or selected from any bifunctional compounds described herein of the invention is capable of treating or preventing a disease or disorder in which a HER family protein plays a role, for example, through the formation of a signaling dimer between EGFR, Her2, or Her4 and Her3. A bifunctional compound of any of the formulae described herein, or selected from any bifunctional compounds described herein of the invention is also capable of treating or preventing a disease or disorder in which Her3 plays a role. In one embodiment, Her3 plays a role through dimer formation with other HER family proteins, such as EGFR, Her2, or Her4. In yet another embodiment, Her3 plays a role by being overexpressed, and is thus deregulated with a bifunctional compound selected from Formula X, Y, I, and II.

Modulation of a HER family protein through UPP-mediated degradation by a bifunctional compound of the application, such as those described herein, provides a suitable approach to the treatment, prevention, or amelioration of diseases or disorders in which a HER family protein plays a role. Further, modulation of a HER family protein through UPP-mediated degradation by a bifunctional compound of the application, such as those described herein, allows the healthcare provider the ability to treat, prevent, or ameliorate diseases or disorders in which a HER family protein is deregulated. In one embodiment, the bifunctional compounds of the application modulate a HER family protein with lower kinase activity relative to EGFR, Her2, and/or Her4 through UPP-mediated degradation. In a further embodiment, the bifunctional compounds of the application modulate the Her3 protein through UPP-mediated degradation.

In one embodiment, a bifunctional compound of any of the formulae described herein, or selected from any bifunctional compounds described herein of the invention is more efficacious in treating a disease or condition than the Targeting Ligand when the Targeting Ligand is administered alone or not bonded to a Linker and a Degron. In one embodiment, a bifunctional compound of any of the formulae described herein, or selected from any bifunctional compounds described herein of the invention is more capable of treating a disease or condition resistant to the Targeting Ligand than the Targeting Ligand when the Targeting Ligand is administered alone or not bonded to a Linker and a Degron. In one embodiment the disease or condition is cancer.

In one embodiment, a bifunctional compound of any of the formulae described herein, or selected from any bifunctional compounds described herein of the invention is capable of modulating or decreasing the amount of a HER family protein and thus is useful in treating a disease or condition in which the HER family protein plays a role. In one embodiment, the bifunctional compounds of the application modulate a HER family protein with lower kinase activity relative to EGFR, Her2, and/or Her4. In a further embodiment, the bifunctional compounds of the application modulate the Her3 protein. In one embodiment, the disease or condition is cancer in which the Her3 protein plays a role.

In one embodiment, the bifunctional compound of the invention that is more efficacious in treating a disease or condition or is more capable of treating a disease or condition resistant to the Targeting Ligand than when the Targeting Ligand is administered alone or when not bonded to a Linker and a Degron, is more potent in inhibiting the growth of cells or decreasing the viability of cells than the Targeting Ligand when the Targeting Ligand is administered alone or not bonded to a Linker and a Degron. In a further embodiment, the cells are cancer cells. In one embodiment, the bifunctional compound inhibits the growth of cells or decreases the viability at an Eₘₐₓ that is lower than the Eₘₐₓ of the Targeting Ligand when the Targeting Ligand is administered alone or not bonded to a Linker and a Degron for inhibiting the growth or decreasing the viability of the cells. In a further embodiment the cells are cancer cells. In one embodiment, the Eₘₐₓ of the bifunctional compound is at most 90%, 80%, 70%, 60%, 50%, 40%, 30%, 20%, 10%, 8%, 5%, 4%, 3%, 2%, or 1% of the Eₘₐₓ of the Targeting Ligand. In one embodiment, the Eₘₐₓ of the bifunctional compound is at most 50%, 40%, 30%, 20%, 10%, 8%, 5%, 4%, 3%, 2%, or 1% of the Eₘₐₓ of the Targeting Ligand. In one embodiment, the Eₘₐₓ of the bifunctional compound is at most 90%, 80%, 70%, 60%, 50%, 40%, 30%, 20%, or 10% of the Eₘₐₓ of the Targeting Ligand.

In one embodiment, the bifunctional compound inhibits the growth of cells or decreases the viability of cells at an IC₅₀ that is lower than the IC₅₀ of the Targeting Ligand when the Targeting Ligand is administered alone or not bonded to a Linker and a Degron for inhibiting the growth or decreasing the viability of the cells. In a further embodiment, the cells are cancer cells.

In one embodiment, the IC₅₀ of the bifunctional compound is at most 90%, 80%, 70%, 60%, 50%, 40%, 30%, 20%, 10%, 8%, 5%, 4%, 3%, 2%, 1%, 0.8%, 0.5%, 0.4%, 0.3%, 0.2%, or 0.1% of the IC₅₀ of the Targeting Ligand. In one embodiment, the IC₅₀ of the bifunctional compound is at most 50%, 40%, 30%, 20%, 10%, 8%, 5%, 4%, 3%, 2%, 1%, 0.8%, 0.5%, 0.4%, 0.3%, 0.2%, or 0.1% of the IC₅₀ of the Targeting Ligand. In one embodiment, the IC₅₀ of the bifunctional compound is at most 30%, 20%, 10%, 8%, 5%, 4%, 3%, 2%, 1%, 0.8%, 0.5%, 0.4%, 0.3%, 0.2%, or 0.1% of the IC₅₀ of the Targeting Ligand. In one embodiment, the IC₅₀ of the bifunctional compound is at most 10%, 8%, 5%, 4%, 3%, 2%, 1%, 0.8%, 0.5%, 0.4%, 0.3%, 0.2%, or 0.1% of the IC₅₀ of the Targeting Ligand. In one embodiment, the IC₅₀ of the bifunctional compound is at most 5%, 4%, 3%, 2%, 1%, 0.8%, 0.5%, 0.4%, 0.3%, 0.2%, or 0.1% of the IC₅₀ of the Targeting Ligand. In one embodiment, the IC₅₀ of the bifunctional compound is at most 2%, 1%, 0.8%, 0.5%, 0.4%, 0.3%, 0.2%, or 0.1% of the IC₅₀ of the Targeting Ligand. In one embodiment, the IC₅₀ of the bifunctional compound is at most 1%, 0.8%, 0.5%, 0.4%, 0.3%, 0.2%, or 0.1% of the IC₅₀ of the Targeting Ligand. In one embodiment, the compounds of the invention are useful as anticancer agents, and thus may be useful in the treatment of cancer, by effecting tumor cell death or inhibiting the growth of tumor cells. In certain exemplary embodiments, the disclosed anticancer agents are useful in the treatment of cancers and other proliferative disorders, including, but not limited to breast cancer, cervical cancer, colon and rectal cancer, leukemia, lung cancer, non-small cell lung cancer, melanoma, multiple myeloma, non-Hodgkin's lymphoma, ovarian cancer, pancreatic cancer, prostate cancer, gastric cancer, leukemias, including but not limited to myeloid, lymphocytic, myelocytic and lymphoblastic leukemias, malignant melanomas, and T-cell lymphoma.

### Definitions

Listed below are definitions of various terms used in this application. These definitions apply to the terms as they are used throughout this specification and claims, unless otherwise limited in specific instances, either individually or as part of a larger group.

The term “alkyl,” as used herein, refers to saturated, straight or branched-chain hydrocarbon radicals containing, in certain embodiments, between one and six carbon atoms. Examples of C₁-C₆ alkyl radicals include, but are not limited to, methyl, ethyl, propyl, isopropyl, n-butyl, tert-butyl, neopentyl, and n-hexyl radicals.

The term “alkenyl,” as used herein, denotes a monovalent group derived from a hydrocarbon moiety containing, in certain embodiments, from two to six carbon atoms having at least one carbon-carbon double bond. The double bond may or may not be the point of attachment to another group. Alkenyl groups include, but are not limited to, for example, ethenyl, propenyl, butenyl, 1-methyl-2-buten-1-yl and the like.

The term “alkoxy” refers to an —O-alkyl radical.

The terms “hal,” “halo,” and “halogen,” as used herein, refer to an atom selected from fluorine, chlorine, bromine and iodine.

The term “cancer” includes, but is not limited to, the following cancers: epidermoid oral: buccal cavity, lip, tongue, mouth, pharynx; cardiac: sarcoma (angiosarcoma, fibrosarcoma, rhabdomyosarcoma, liposarcoma), myxoma, rhabdomyoma, fibroma, lipoma, and teratoma; lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal: esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, rectum; genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver: hepatoma (hepatocellular carcinoma), cholangiocarcinoma, hepatoblastoma, angiosarcoma, hepatocellular adenoma, hemangioma, biliary passages; bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma) hairy cell; lymphoid disorders; Skin: malignant melanoma, basal cell carcinoma, squamous cell carcinoma, Karposi's sarcoma, keratoacanthoma, moles dysplastic nevi, lipoma, angioma, dermatofibroma, keloids, psoriasis, Thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; and Adrenal glands: neuroblastoma. Thus, the term “cancerous cell” as provided herein, includes a cell afflicted by any one of the above-identified conditions.

The term “EGFR” herein refers to epidermal growth factor receptor kinase.

The term “HER” or “Her” herein refers to human epidermal growth factor receptor kinase.

The term “targeted protein(s)” is used interchangeably with “target protein(s)”, unless the context clearly dictates otherwise. In one embodiment, a “targeted protein” is a HER family protein, such as Her3.

The term “subject” as used herein refers to a mammal. A subject therefore refers to, for example, dogs, cats, horses, cows, pigs, guinea pigs, and the like. Preferably the subject is a human. When the subject is a human, the subject may be referred to herein as a patient.

The terms “disease(s)”, “disorder(s)”, and “condition(s)” are used interchangeably, unless the context clearly dictates otherwise.

“Treat”, “treating” and “treatment” refer to a method of alleviating or abating a disease and/or its attendant symptoms.

As used herein, “preventing” or “prevent” describes reducing or eliminating the onset of the symptoms or complications of the disease, condition or disorder.

The term “therapeutically effective amount” of a compound or pharmaceutical composition of the application, as used herein, means a sufficient amount of the compound or pharmaceutical composition so as to decrease the symptoms of a disorder in a subject. As is well understood in the medical arts a therapeutically effective amount of a compound or pharmaceutical composition of this application will be at a reasonable benefit/risk ratio applicable to any medical treatment. It will be understood, however, that the total daily usage of the compounds and compositions of the invention will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

As used herein, the term “pharmaceutically acceptable salt” refers to those salts of the compounds formed by the process of the invention which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the application, or separately by reacting the free base or acid function with a suitable acid or base.

Examples of pharmaceutically acceptable salts include, but are not limited to, nontoxic acid addition salts: salts formed with inorganic acids such as hydrochloric acid, hydrobromic acid, phosphoric acid, sulfuric acid and perchloric acid, or with organic acids such as acetic acid, maleic acid, tartaric acid, citric acid, succinic acid or malonic acid. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, /7-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Combinations of substituents and variables envisioned by this application are only those that result in the formation of stable compounds. The term “stable”, as used herein, refers to compounds which possess stability sufficient to allow manufacture and which maintain the integrity of the compound for a sufficient period of time to be useful for the purposes detailed herein. In one embodiment the purpose is therapeutic administration to a subject. In one embodiment the purpose is prophylactic administration to a subject.

When any variable selected from X^{T}, Tn1, Tn2, R^{T1}, R^{T2}, R^{T5}, R^{T6}, R^{T7}, R^{TN1}, R^{TN2} X, Y, R¹, R^{2′}, R², R³, R^{3′}, R⁵, R⁶, R⁷, R⁸, Dn1, Dn2, Dn3, p1, p2, p3, W, Q, and Z occurs more than one time in any constituent or formula for a compound, its definition at each occurrence is independent of its definition at every other occurrence. Thus, for example, if a group is shown to be substituted with one or more R¹ moieties, then R¹ at each occurrence is selected independently from the definition of R¹. Also, combinations of substituents and/or variables are permissible, but only if such combinations result in stable compounds within a designated atom's normal valency.

In addition, some of the compounds of this application have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms, and other stereoisomeric forms that may be defined, in terms of absolute stereochemistry, as (R)- or (S)-, or as (D)- or (L)- for amino acids. When the compounds described herein contain olefinic double bonds or other centers of geometric asymmetry, and unless specified otherwise, it is intended that the compounds include both E and Z geometric isomers. The configuration of any carbon-carbon double bond appearing herein is selected for convenience only and is not intended to designate a particular configuration unless the text so states; thus a carbon-carbon double bond depicted arbitrarily herein as trans may be cis, trans, or a mixture of the two in any proportion. All such isomeric forms of such compounds are expressly included in the invention.

Optical isomers may be prepared from their respective optically active precursors by the procedures described herein, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981).

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

A carbon atom bonded to four non-identical substituents is termed a “chiral center”.

“Chiral isomer” means a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Furthermore, the structures and other compounds discussed in this application include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose. Common tautomeric pairs are: ketone-enol, amide-nitrile, lactam-lactim, amide-imidic acid tautomerism in heterocyclic rings, nucleobases such as guanine, thymine and cytosine, amine-enamine and enamine-enamine. The compounds of this application may also be represented in multiple tautomeric forms, in such instances, the application expressly includes all tautomeric forms of the compounds described herein. Alkylation of a ring system may result in alkylation at multiple sites, and the application expressly includes all such reaction products.

In the invention, the structural formula of the compound represents a certain isomer for convenience in some cases, but the invention includes all isomers, such as geometrical isomers, optical isomers based on an asymmetrical carbon, stereoisomers, tautomers, and the like.

Additionally, the compounds of the invention, for example, the salts of the compounds, can exist in either hydrated or unhydrated (the anhydrous) form or as solvates with other solvent molecules. Non-limiting examples of hydrates include monohydrates, dihydrates, etc. Non-limiting examples of solvates include ethanol solvates, acetone solvates, etc.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

### Pharmaceutical Compositions

In another aspect, the application provides a pharmaceutical composition comprising a therapeutically effective amount of a bifunctional compound of the invention or an enantiomer, diastereomer, stereoisomer, or pharmaceutically acceptable salt thereof, and a pharmaceutically acceptable carrier.

Bifunctional compounds of the application can be administered as pharmaceutical compositions by any conventional route, in particular enterally, orally in the form of tablets or capsules, or parenterally in the form of injectable solutions or suspensions, or topically in the form of lotions, gels, ointments or creams, or in a nasal or suppository form. Pharmaceutical compositions comprising a compound of the invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, including but not limited to lactose, dextrose, sucrose, mannitol, sorbitol, cellulose and/or glycine; b) lubricants, including but not limited to silica, talcum, stearic acid, its magnesium or calcium salt and/or polyethyleneglycol; for tablets also c) binders, including but not limited to magnesium aluminum silicate, starch paste, gelatin, tragacanth, methylcellulose, sodium carboxymethylcellulose and or polyvinylpyrrolidone; if desired d) disintegrants, including but not limited to starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions.

The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, such as to the skin and eyes, are preferably aqueous solutions, ointments, creams or gels well-known in the art. Such may contain solubilizers, stabilizers, tonicity enhancing agents, buffers and preservatives.

The pharmaceutical compositions of the invention comprise a therapeutically effective amount of a compound of the invention formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylenepolyoxy propylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes, oils such as peanut oil, cottonseed oil; safflower oil; sesame oil; olive oil; corn oil and soybean oil; glycols such a propylene glycol or polyethylene glycol; esters such as ethyl oleate and ethyl laurate, agar; buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this application can be administered to humans and other animals orally, rectally, parenterally, intracisternally, intravaginally, intraperitoneally, topically (as by powders, ointments, or drops), buccally, or as an oral or nasal spray.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous, or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this application with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, including but not limited to tableting lubricants and other tableting aids such a magnesium stearate and microcrystalline cellulose. In the case of capsules, tablets and pills, the dosage forms may also comprise buffering agents.

Dosage forms for topical or transdermal administration of a compound of this application include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this application.

The ointments, pastes, creams and gels may contain, in addition to an active compound of this application, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this application, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Methods of the Application

In another aspect, the application provides a method for modulating or decreasing the amount of a targeted protein by administering a therapeutically effective amount of a bifunctional compound or a pharmaceutical composition of the application to a subject in need thereof. In one embodiment the targeted protein is a HER family protein. In a further embodiment the targeted protein is Her3. The invention also provides a method for treating or preventing a disease or condition which is modulated by a targeted protein by administering a therapeutically effective amount of a bifunctional compound or a pharmaceutical composition of the application to a subject in need thereof. In one embodiment the disease or condition is a cancer modulated by a targeted protein. In a further embodiment, the targeted protein is a HER family protein. In a further embodiment, the disease or condition is a cancer modulated by Her3.

In some embodiments, the disease is mediated by a HER family protein. In one embodiment a HER family protein plays a role in the initiation or development of the disease. In further embodiments, the HER family protein is a Her protein that has a lower kinase activity relative to EGFR, Her2, and/or Her4. In further embodiments, the HER family protein is Her3.

In certain embodiments, the disease is cancer or a proliferation disease.

In further embodiments, the disease is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors.

In other embodiments, the disease is inflammation, arthritis, rheumatoid arthritis, spondyiarthropathies, gouty arthritis, osteoarthritis, juvenile arthritis, and other arthritic conditions, systemic lupus erthematosus (SLE), skin-related conditions, psoriasis, eczema, burns, dermatitis, neuroinflammation, allergy, pain, neuropathic pain, fever, pulmonary disorders, lung inflammation, adult respiratory distress syndrome, pulmonary sarcoisosis, asthma, silicosis, chronic pulmonary inflammatory disease, and chronic obstructive pulmonary disease (COPD), cardiovascular disease, arteriosclerosis, myocardial infarction (including post-myocardial infarction indications), thrombosis, congestive heart failure, cardiac reperfusion injury, as well as complications associated with hypertension and/or heart failure such as vascular organ damage, restenosis, cardiomyopathy, stroke including ischemic and hemorrhagic stroke, reperfusion injury, renal reperfusion injury, ischemia including stroke and brain ischemia, and ischemia resulting from cardiac/coronary bypass, neurodegenerative disorders, liver disease and nephritis, gastrointestinal conditions, inflammatory bowel disease, Crohn's disease, gastritis, irritable bowel syndrome, ulcerative colitis, ulcerative diseases, gastric ulcers, viral and bacterial infections, sepsis, septic shock, gram negative sepsis, malaria, meningitis, HIV infection, opportunistic infections, cachexia secondary to infection or malignancy, cachexia secondary to acquired immune deficiency syndrome (AIDS), AIDS, ARC (AIDS related complex), pneumonia, herpes virus, myalgias due to infection, influenza, autoimmune disease, graft vs. host reaction and allograft rejections, treatment of bone resorption diseases, osteoporosis, multiple sclerosis, cancer, leukemia, lymphoma, colorectal cancer, brain cancer, bone cancer, epithelial call-derived neoplasia (epithelial carcinoma), basal cell carcinoma, adenocarcinoma, gastrointestinal cancer, lip cancer, mouth cancer, esophageal cancer, small bowel cancer, stomach cancer, colon cancer, liver cancer, bladder cancer, pancreas cancer, ovarian cancer, cervical cancer, lung cancer, breast cancer, skin cancer, squamus cell and/or basal cell cancers, prostate cancer, renal cell carcinoma, and other known cancers that affect epithelial cells throughout the body, chronic myelogenous leukemia (CML), acute myeloid leukemia (AML) and acute promyelocytic leukemia (APL), angiogenesis including neoplasia, metastasis, central nervous system disorders, central nervous system disorders having an inflammatory or apoptotic component, Alzheimer's disease, Parkinson's disease, Huntington's disease, amyotrophic lateral sclerosis, spinal cord injury, and peripheral neuropathy, or B-Cell Lymphoma.

In further embodiments, the disease is inflammation, arthritis, rheumatoid arthritis, spondylarthropathies, gouty arthritis, osteoarthritis, juvenile arthritis, and other arthritic conditions, systemic lupus erthematosus (SLE), skin-related conditions, psoriasis, eczema, dermatitis, pain, pulmonary disorders, lung inflammation, adult respiratory distress syndrome, pulmonary sarcoisosis, asthma, chronic pulmonary inflammatory disease, and chronic obstructive pulmonary disease (COPD), cardiovascular disease, arteriosclerosis, myocardial infarction (including post-myocardial infarction indications), congestive heart failure, cardiac reperfusion injury, inflammatory bowel disease, Crohn's disease, gastritis, irritable bowel syndrome, leukemia or lymphoma.

In another aspect, the application provides a method of treating or preventing a disease wherein the cells comprise a deregulated HER family protein, comprising administering to a subject in need thereof a therapeutically effective amount of a bifunctional compound or a pharmaceutical composition of the application to a subject in need thereof. In one embodiment the disease is cancer. In a further embodiment, the cancer cells comprise deregulated Her3 protein.

In certain embodiments, the application provides a method of treating any of the disorders described herein, wherein the subject is a human. In certain embodiments, the application provides a method of preventing any of the disorders described herein, wherein the subject is a human.

In another aspect, the application provides a bifunctional compound or a pharmaceutical composition thereof for use in the manufacture of a medicament for treating or preventing a disease which is modulated by a targeted protein. In one embodiment the targeted protein is a HER family protein. In a further embodiment, the HER family protein is Her3.

In still another aspect, the application provides the use of a bifunctional compound or a pharmaceutical composition thereof in the treatment or prevention of a disease which is modulated by a targeted protein. In one embodiment the targeted protein is a HER family protein. In a further embodiment, the HER family protein is Her3.

The compounds and compositions of this application are particularly useful for treating or lessening the severity of a disease, condition, or disorder where a protein kinase is implicated in the disease, condition, or disorder. In one embodiment the protein kinase is a HER family protein. In a further embodiment, the protein kinase is Her3.

In one aspect, the invention provides a method for treating or lessening the severity of a disease, condition, or disorder where a protein kinase is implicated in the disease state. In another aspect, the invention provides a method for treating or lessening the severity of a kinase disease, condition, or disorder where inhibition of enzymatic activity is implicated in the treatment of the disease. In another aspect, this application provides a method for treating or lessening the severity of a disease, condition, or disorder with compounds that inhibit enzymatic activity by interfering with or blocking dimer formation between HER family proteins, such as dimer formation between EGFR, Her2, or Her4 and Her3 through modulation of the amount of a HER family protein. In one embodiment the HER family protein is Her3.

In some embodiments, the method of the application is used to treat or prevent a condition selected from autoimmune diseases, inflammatory diseases, proliferative and hyperproliferative diseases, immunologically-mediated diseases, bone diseases, metabolic diseases, neurological and neurodegenerative diseases, cardiovascular diseases, hormone related diseases, allergies, asthma, and Alzheimer's disease. In other embodiments, the condition is selected from a proliferative disorder and a neurodegenerative disorder.

The term “cancer” refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers such as oral, laryngeal, nasopharyngeal and esophageal, genitourinary cancers, such as prostate, bladder, renal, uterine, ovarian, and testicular, lung cancer, such as small-cell and non-small cell, breast cancer, pancreatic cancer, melanoma and other skin cancers, stomach cancer, brain tumors, tumors related to Gorlin's syndrome, including but not limited to medulloblastoma and meningioma, and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in preventing, treating and studying are, for example, colon carcinoma, familiary adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma. In one aspect of the application, the invention provides for the use of one or more compounds of the application in the manufacture of a medicament for the treatment of cancer, including without limitation the various types of cancer disclosed herein.

Compounds and compositions of the application can be administered in therapeutically effective amounts in a combinational therapy with one or more therapeutic agents (pharmaceutical combinations) or modalities. In one embodiment, a second agent modulates one or more other HER family proteins. In one embodiment, a second agent inhibits one or more other HER family proteins. In a further embodiment, the second agent is an anti-proliferative, anti-cancer, immunomodulatory or anti-inflammatory substance. Where the compounds of the application are administered in conjunction with other therapies, dosages of the co-administered compounds will of course vary depending on the type of co-drug employed, on the specific drug employed, on the condition being treated and so forth.

### Combination Therapy

In one aspect, a treatment regimen is provided comprising the administration of a compound selected from Formula X, Y, I, and II, or a pharmaceutically acceptable composition, salt, isotopic analog (such as a deuterated derivative), or prodrug thereof in combination or in alternation with at least one additional therapeutic agent. The combinations and/or alternations disclosed herein can be administered for beneficial, additive, or synergistic effect in the treatment of abnormal cellular proliferative disorders.

In one aspect of this embodiment, the second active compound is an immune modulator, including but not limited to a checkpoint inhibitor. Checkpoint inhibitors for use in the methods described herein include, but are not limited to PD-1 inhibitors, PD-L1 inhibitors, PD-L2 inhibitors, CTLA-4 inhibitors, LAG-3 inhibitors, TIM-3 inhibitors, and V-domain Ig suppressor of T-cell activation (VISTA) inhibitors, or combination thereof.

In one embodiment, the checkpoint inhibitor is a PD-1 inhibitor that blocks the interaction of PD-1 and PD-L1 by binding to the PD-1 receptor, and in turn inhibits immune suppression. In one embodiment, the checkpoint inhibitor is a PD-1 checkpoint inhibitor selected from nivolumab, pembrolizumab, pidilizumab, AMP-224 (AstraZeneca and MedImmune), PF-06801591 (Pfizer), MEDI0680 (AstraZeneca), PDR001 (Novartis), REGN2810 (Regeneron), SHR-12-1 (Jiangsu Hengrui Medicine Company and Incyte Corporation), TSR-042 (Tesaro), and the PD-L1/VISTA inhibitor CA-170 (Curis Inc.).

In one embodiment, the checkpoint inhibitor is a PD-L1 inhibitor that blocks the interaction of PD-1 and PD-L1 by binding to the PD-L1 receptor, and in turn inhibits immune suppression. PD-L1 inhibitors include, but are not limited to, avelumab, atezolizumab, durvalumab, KN035, and BMS-936559 (Bristol-Myers Squibb).

In one aspect of this embodiment, the checkpoint inhibitor is a CTLA-4 checkpoint inhibitor that binds to CTLA-4 and inhibits immune suppression. CTLA-4 inhibitors include, but are not limited to, ipilimumab, tremelimumab (AstraZeneca and MedImmune), AGEN1884 and AGEN2041 (Agenus).

In another embodiment, the checkpoint inhibitor is a LAG-3 checkpoint inhibitor. Examples of LAG-3 checkpoint inhibitors include, but are not limited to, BMS-986016 (Bristol-Myers Squibb), GSK2831781 (GlaxoSmithKline), IMP321 (Prima BioMed), LAG525 (Novartis), and the dual PD-1 and LAG-3 inhibitor MGD013 (MacroGenics). In yet another aspect of this embodiment, the checkpoint inhibitor is a TIM-3 checkpoint inhibitor. A specific TIM-3 inhibitor includes, but is not limited to, TSR-022 (Tesaro).

In another embodiment, the compound for use in combination therapy is a LAG-3 targeting ligand. In another embodiment, the compound for use in combination therapy is a TIM-3 targeting ligand. In another embodiment, the compound for use in combination therapy is a aromatase inhibitor. In another embodiment, the compound for use in combination therapy is a progestin receptor targeting ligand. In another embodiment, the compound for use in combination therapy is a CYP3A4 targeting ligand. In another embodiment, the compound for use in combination therapy is a TORC1 or TORC2 targeting ligand.

In specific embodiments, the treatment regimen includes the administration of a compound selected from Formula X, Y, I, and II, or a pharmaceutically acceptable composition, salt, isotopic analog, or prodrug thereof in combination or alternation with at least one additional kinase inhibitor. In one embodiment, the at least one additional kinase inhibitor is selected from a phosphoinositide 3-kinase (PI3K) inhibitor, a Bruton's tyrosine kinase (BTK) inhibitor, a cyclin-dependent kinase inhibitor, or a spleen tyrosine kinase (Syk) inhibitor, or a combination thereof.

In one embodiment, the additional active agent is the small molecule BET inhibitor, MK-8628 (CAS 202590-98-5) (6H-thieno(3,2-f)-(1,2,4)triazolo(4,3-a)-(1,4)diazepine-6-acetamide, 4-(4-chlorophenyl)-N-(4-hydroxyphenyl)2,3,9-trimethyl, (6S).

In one embodiment, a compound selected from Formula X, Y, I, and II, or a pharmaceutically acceptable composition, salt, isotopic analog, or prodrug thereof is combined in a dosage form with the PIk3 inhibitor.

PI3k inhibitors that may be used in the present invention are well known. Examples of PI3 kinase inhibitors include but are not limited to Wortmannin, demethoxyviridin, perifosine, idelalisib, Pictilisib, Palomid 529, ZSTK474, PWT33597, CUDC-907, and AEZS-136, duvelisib, GS-9820, GDC-0032 (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2S)—N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide), TGX-221 ((+)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-((l-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluor0-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[1-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), Buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methylsulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF 1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N′-[4-(4,6-di-4-morpholinyl-1,3,5-triazin-2-yl)phenyl]urea), LY3023414, BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3 aR,6E,9S,9aR,1 OR, 11 aS)-6-[[bis(prop-2-enyl)amino]methylidene]-5-hydroxy-9-(methoxymethyl)-9a, 11a-dimethyl-1,4,7-trioxo-2,3,3a,9,10,11-hexahydroindeno[4,5h]isochromen-10-yl] acetate (also known as sonolisib)).

BTK inhibitors for use in the present invention are well known. Examples of BTK inhibitors include ibrutinib (also known as PCI-32765)(Imbruvica™)(1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (see US Patent Publication No 2011/0117073, incorporated herein in its entirety), Dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-5604-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one), and other molecules capable of inhibiting BTK activity, for example those BTK inhibitors disclosed in Akinleye et ah, Journal of Hematology & Oncology, 2013, 6:59, the entirety of which is incorporated herein by reference. In one embodiment, a compound selected from Formula X, Y, I, and II, or a pharmaceutically acceptable composition, salt, isotopic analog, or prodrug thereof is combined in a dosage form with the BTK inhibitor.

Syk inhibitors for use in the present invention are well known, and include, for example, Cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), R09021 (6-[(1R,2S)-2-Amino-cyclohexylamino]-4-(5,6-dimethyl-pyridin-2-ylamino)-pyridazine-3-carboxylic acid amide), imatinib (Gleevec; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), YM193306 (see Singh et al. Discovery and Development of Spleen Tyrosine Kinase (SYK) Inhibitors, J. Med. Chem. 2012, 55, 3614-3643), 7-azaindole, piceatannol, ER-27319 (see Singh et al. Discovery and Development of Spleen Tyrosine Kinase (SYK) Inhibitors, J. Med. Chem. 2012, 55, 3614-3643 incorporated in its entirety herein), PRT060318 (see Singh et al. Discovery and Development of Spleen Tyrosine Kinase (SYK) Inhibitors, J. Med. Chem. 2012, 55, 3614-3643 incorporated in its entirety herein), luteolin (see Singh et al. Discovery and Development of Spleen Tyrosine Kinase (SYK) Inhibitors, J. Med. Chem. 2012, 55, 3614-3643 incorporated in its entirety herein), apigenin (see Singh et al. Discovery and Development of Spleen Tyrosine Kinase (SYK) Inhibitors, J. Med. Chem. 2012, 55, 3614-3643 incorporated in its entirety herein), quercetin (see Singh et al. Discovery and Development of Spleen Tyrosine Kinase (SYK) Inhibitors, J. Med. Chem. 2012, 55, 3614-3643 incorporated in its entirety herein), fisetin (see Singh et al. Discovery and Development of Spleen Tyrosine Kinase (SYK) Inhibitors, J. Med. Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein). In one embodiment a compound selected from Formula X, Y, I and II, or a pharmaceutically acceptable composition, salt, isotopic analog, or prodrug thereof is combined in a dosage form with the Syk inhibitor.

In specific embodiments, the method of treatment provided includes the administration of a compound selected from Formula X, Y, I and II, or a pharmaceutically acceptable composition, salt, isotopic analog, or prodrug thereof in combination or alternation with at least one additional chemotherapeutic agent.

In one embodiment, at least one additional chemotherapeutic agent combined or alternated with a compound selected from Formula X, Y, I and II, is a protein cell death-1 (PD-1) inhibitor. PD-1 inhibitors are known in the art, and include, for example, nivolumab (BMS), pembrolizumab (Merck), pidilizumab (CureTech/Teva), AMP-244 (Amplimmune/GSK), BMS-936559 (BMS), and MEDI4736 (Roche/Genentech). In one embodiment, a compound selected from Formula X, Y, I and II, or a pharmaceutically acceptable composition, salt, isotopic analog, or prodrug thereof is combined in a dosage form with the PD-1 inhibitor. In one embodiment the PD-1 inhibitor is pembrolizumab.

In one embodiment, the at least one additional chemotherapeutic agent combined or alternated with a compound selected from Formula X, Y, I and II is a CTLA-4 inhibitor. CTLA-4 inhibitors are known in the art, and include, for example, ipilimumab (Yervoy) marketed by Bristol-Myers Squibb and tremelimumab marketed by Pfizer.

In one embodiment, the at least one additional chemotherapeutic agent combined or alternated with the compound selected from Formula X, Y, I and II is a BET inhibitor. BET inhibitors are known in the art, and include, for example, JQ1, I-BET 151 (a.k.a. GSK1210151A), I-BET 762 (a.k.a. GSK525762), OTX-015 (a.k.a. MK-8268, IUPAC 6H-Thieno[3,2-f][1,2,4]triazolo[4,3-a][1,4]diazepine-6-acetamide, 4-(4-chlorophenyl)-N-(4-hydroxyphenyl)-2,3,9-trimethyl-), TEN-010, CPI-203, CPI-0610, RVX-208, and LY294002. In one embodiment the BET inhibitor used in combination or alternation with a compound selected from Formula X, Y, I and II for treatment of a tumor or cancer is JQ1 ((S)-tert-butyl 2-(4-(4-chlorophenyl)-2,3,9-trimethyl-6H-thieno[3,2-f][1,2,4]triazolo[4,3-a][1,4]diazepin-6-yl)acetate). In an alternative embodiment the BET inhibitor used in combination or alternation with a compound selected from Formula X, Y, I and II for treatment of a tumor or cancer is I-BET 151 (2H-Imidazo[4,5-c]quinolin-2-one, 7-(3,5-dimethyl-4-isoxazolyl)-1,3-dihydro-8-methoxy-1-[(1R)-1-(2-pyridinyl) ethyl]-).

In one embodiment, the at least one additional chemotherapeutic agent combined or alternated with the compound selected from Formula X, Y, I and II is a MEK inhibitor. MEK inhibitors for use in the present invention are well known, and include, for example, tametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinob (6-(4-bromo-2-chloroanilino)-7-fluoro-N-(2-hydroxyethoxy)-3-methylbenzimidazole-5-carboxamide), pimasertib/AS703026/MSC 1935369 ((S)—N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol), refametinib/BAY869766/RDEAl 19 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3-d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6-carboxamide), R05126766 (3-[[3-Fluoro-2-(methyl sulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2yl)methyl)benzamide), or AZD8330 (2-((2-fluoro-4-iodophenyl)amino)-N-(2 hydroxyethoxy)-1, and 5-dimethyl-6-oxo-1,6-dihydropyridine-3-carboxamide). In one embodiment, a compound selected from Formula X, Y, I and II, or a pharmaceutically acceptable composition, salt, isotopic analog, or prodrug thereof is combined in a dosage form with the MEK inhibitor.

In one embodiment, the at least one additional chemotherapeutic agent combined or alternated with the compound of the present invention is a Raf inhibitor. Raf inhibitors for use in the present invention are well known, and include, for example, Vemurafinib (N-[3-[[5-(4-Chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide; 4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-Bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), and Sorafenib N-Oxide (4-[4-[[[[4-Chloro-3 (trifluoroMethyl)phenyl]aMino]carbonyl]aMino]phenoxy]-N-Methyl-2pyridinecarboxaMide 1-Oxide). In one embodiment, a compound selected from Formula X, Y, I and II, or a pharmaceutically acceptable composition, salt, isotopic analog, or prodrug thereof is combined in a dosage form with the Raf inhibitor.

In one embodiment, the at least one additional chemotherapeutic agent combined or alternated with the compound selected from Formula X, Y, I and II, is a B-cell lymphoma 2 (Bcl-2) protein inhibitor. BCL-2 inhibitors are known in the art, and include, for example, ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenyl sulfanylbutan-2-yl]amino]-3-nitrophenyl]sulfonylbenzamide), ABT-263 ((R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3((trifluoromethyl)sulfonyl)phenyl)sulfonyl)benzamide), GX15-070 (obatoclax mesylate, (2Z)-2-[(5Z)-5-[(3,5-dimethyl-1H-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-Dimethylethyl)phenyl]sulfonyl]phenyl]-2,3,4-trihydroxy-5-[[2-(1-methylethyl)phenyl]methyl]benzamide), Apogossypolone (ApoG2), or G3139 (Oblimersen). In one embodiment, a compound selected from Formula X, Y, I and II, or a pharmaceutically acceptable composition, salt, isotopic analog, or prodrug thereof is combined in a dosage form with the at least one BCL-2 inhibitor. In one embodiment the at least one BCL-2 inhibitor is ABT-199 (Venetoclax).

In one embodiment, the treatment regimen includes the administration of a compound selected from Formula X, Y, I and II, or a pharmaceutically acceptable composition, salt, isotopic analog, or prodrug thereof in combination or alternation with at least one additional chemotherapeutic agent selected from, but are not limited to, Imatinib mesylate (Gleevac), Dasatinib (Sprycel), Nilotinib (Tasigna), Bosutinib (Bosulif), Trastuzumab (Herceptin), Pertuzumab (Perjeta™), Lapatinib (Tykerb), Gefitinib (Iressa), Erlotinib (Tarceva), Cetuximab (Erbitux), Panitumumab (Vectibix), Vandetanib (Caprelsa), Vemurafenib (Zelboraf), Vorinostat (Zolinza), Romidepsin (Istodax), Bexarotene (Tagretin), Alitretinoin (Panretin), Tretinoin (Vesanoid), Carfilizomib (Kyprolis™), Pralatrexate (Folotyn), Bevacizumab (Avastin), Ziv-aflibercept (Zaltrap), Sorafenib (Nexavar), Sunitinib (Sutent), Pazopanib (Votrient), Regorafenib (Stivarga), and Cabozantinib (Cometriq™).

In some embodiments, the pharmaceutical combination or composition described herein can be administered to the subject in combination or further combination with other chemotherapeutic agents for the treatment of a tumor or cancer. If convenient, the pharmaceutical combination or composition described herein can be administered at the same time as another chemotherapeutic agent, in order to simplify the treatment regimen. In some embodiments, the pharmaceutical combination or composition and the other chemotherapeutic can be provided in a single formulation. In one embodiment, the use of the pharmaceutical combination or composition described herein is combined in a therapeutic regime with other agents. Such agents may include, but are not limited to, tamoxifen, midazolam, letrozole, bortezomib, anastrozole, goserelin, an mTOR inhibitor, a PI3 kinase inhibitor as described above, a dual mTOR-PI3K inhibitor, a MEK inhibitor as described above, a RAS inhibitor, ALK inhibitor, an HSP inhibitor (for example, HSP70 and HSP 90 inhibitor, or a combination thereof), a BCL-2 inhibitor as described above, apopototic inducing compounds, an AKT inhibitor, including but not limited to, MK-2206 (1,2,4-Triazolo[3,4-f][1,6]naphthyridin-3 (2H)-one, 8-[4-(1-aminocyclobutyl)phenyl]-9-phenyl-), GSK690693, Perifosine, (KRX-0401), GDC-0068, Triciribine, AZD5363, Honokiol, PF-04691502, and Miltefosine, a PD-1 inhibitor as described above including but not limited to, Nivolumab, CT-011, MK-3475, BMS936558, and AMP-514 or a FLT-3 inhibitor, including but not limited to, P406, Dovitinib, Quizartinib (AC220), Amuvatinib (MP-470), Tandutinib (MLN518), ENMD-2076, and KW-2449, or a combination thereof. Examples of mTOR inhibitors include but are not limited to rapamycin and its analogs, everolimus (Afinitor), temsirolimus, ridaforolimus, sirolimus, and deforolimus. Examples of RAS inhibitors include but are not limited to Reolysin and siG12D LODER. Examples of ALK inhibitors include but are not limited to Crizotinib, AP26113, and LDK378. HSP inhibitors include but are not limited to Geldanamycin or 17-N-Allylamino-17-demethoxygeldanamycin (17AAG), and Radicicol. In a particular embodiment, a compound described herein is administered in combination with letrozole and/or tamoxifen. Other chemotherapeutic agents that can be used in combination with the compounds described herein include, but are not limited to, chemotherapeutic agents that do not require cell cycle activity for their anti-neoplastic effect.

In one embodiment, the treatment regimen includes the administration of a compound selected from Formula X, Y, I and II, or a pharmaceutically acceptable composition, salt, isotopic analog, or prodrug thereof in combination or alternation with at least one additional therapy. The second therapy can be an immunotherapy. As discussed in more detail below, the combination agent can be conjugated to an antibody, radioactive agent, or other targeting agent that directs the active compound as described herein to the diseased or abnormally proliferating cell. In another embodiment, the pharmaceutical combination or composition is used in combination with another pharmaceutical or a biologic agent (for example an antibody) to increase the efficacy of treatment with a combined or a synergistic approach. In an embodiment, the pharmaceutical combination or composition can be used with T-cell vaccination, which typically involves immunization with inactivated autoreactive T cells to eliminate a cancer cell population as described herein. In another embodiment, the pharmaceutical combination or composition is used in combination with a bispecific T-cell Engager (BiTE), which is an antibody designed to simultaneously bind to specific antigens on endogenous T cells and cancer cells as described herein, linking the two types of cells.

In one embodiment, the additional therapy is a monoclonal antibody (MAb). Some MAbs stimulate an immune response that destroys cancer cells. Similar to the antibodies produced naturally by B cells, these MAbs “coat” the cancer cell surface, triggering its destruction by the immune system. For example, bevacizumab targets vascular endothelial growth factor (VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. Similarly, cetuximab and panitumumab target the epidermal growth factor receptor (EGFR), and trastuzumab targets the human epidermal growth factor receptor 2 (HER-2). MAbs that bind to cell surface growth factor receptors prevent the targeted receptors from sending their normal growth-promoting signals. They may also trigger apoptosis and activate the immune system to destroy tumor cells.

Another group of cancer therapeutic MAbs are the immunoconjugates. These MAbs, which are sometimes called immunotoxins or antibody-drug conjugates, consist of an antibody attached to a cell-killing substance, such as a plant or bacterial toxin, a chemotherapy drug, or a radioactive molecule. The antibody latches onto its specific antigen on the surface of a cancer cell, and the cell-killing substance is taken up by the cell. FDA-approved conjugated MAbs that work this way include ado-trastuzumab emtansine, which targets the HER-2 molecule to deliver the drug DM1, which inhibits cell proliferation, to HER-2 expressing metastatic breast cancer cells.

Immunotherapies with T cells engineered to recognize cancer cells via bispecific antibodies (bsAbs) or chimeric antigen receptors (CARs) are approaches with potential to ablate both dividing and non/slow-dividing subpopulations of cancer cells.

Bispecific antibodies, by simultaneously recognizing target antigen and an activating receptor on the surface of an immune effector cell, offer an opportunity to redirect immune effector cells to kill cancer cells. Another approach is the generation of chimeric antigen receptors by fusing extracellular antibodies to intracellular signaling domains. Chimeric antigen receptor-engineered T cells are able to specifically kill tumor cells in a MHC-independent way.

In certain aspects, the additional therapy is another therapeutic agent, for example, an anti-inflammatory agent, a chemotherapeutic agent, a radiotherapeutic agent, or an immunosuppressive agent.

Suitable chemotherapeutic agents include, but are not limited to, a radioactive molecule, a toxin, also referred to as cytotoxin or cytotoxic agent, which includes any agent that is detrimental to the viability of cells, and liposomes or other vesicles containing chemotherapeutic compounds. General anticancer pharmaceutical agents include: Vincristine (Oncovin) or liposomal vincristine (Marqibo), Daunorubicin (daunomycin or Cerubidine) or doxorubicin (Adriamycin), Cytarabine (cytosine arabinoside, ara-C, or Cytosar), L-asparaginase (Elspar) or PEG-L-asparaginase (pegaspargase or Oncaspar), Etoposide (VP-16), Teniposide (Vumon), 6-mercaptopurine (6-MP or Purinethol), Methotrexate, Cyclophosphamide (Cytoxan), Prednisone, Dexamethasone (Decadron), imatinib (Gleevec marketed by Novartis), dasatinib (Sprycel), nilotinib (Tasigna), bosutinib (Bosulif), and ponatinib (Iclusig™). Examples of additional suitable chemotherapeutic agents include but are not limited to 1-dehydrotestosterone, 5-fluorouracil decarbazine, 6-mercaptopurine, 6-thioguanine, actinomycin D, adriamycin, aldesleukin, an alkylating agent, allopurinol sodium, altretamine, amifostine, anastrozole, anthramycin (AMC)), an anti-mitotic agent, cis-dichlorodiamine platinum (II) (DDP) cisplatin), diamino dichloro platinum, anthracycline, an antibiotic, an antimetabolite, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunirubicin HCL, daunorucbicin citrate, denileukin diftitox, Dexrazoxane, Dibromomannitol, dihydroxy anthracin dione, Docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

Suitable immunosuppressive agents include, but are not limited to: calcineurin inhibitors, e.g. a cyclosporin or an ascomycin, e.g. Cyclosporin A (NEORAL), FK506 (tacrolimus), pimecrolimus, a mTOR inhibitor, e.g. rapamycin or a derivative thereof, e.g. Sirolimus (RAPAMUNE), Everolimus (Certican), temsirolimus, zotarolimus, biolimus-7, biolimus-9, a rapalog, e.g. ridaforolimus, azathioprine, campath 1H, a S1P receptor modulator, e.g. fingolimod or an analog thereof, an anti IL-8 antibody, mycophenolic acid or a salt thereof, e.g. sodium salt, or a prodrug thereof, e.g. Mycophenolate Mofetil (CELLCEPT), OKT3 (ORTHOCLONE OKT3), Prednisone, ATGAM, THYMOGLOBULIN, Brequinar Sodium, OKT4, T10B9.A-3A, 33B3.1, 15-deoxyspergualin, tresperimus, Leflunomide ARAVA, CTLAI-Ig, anti-CD25, anti-IL2R, Basiliximab (SIMULECT), Daclizumab (ZENAPAX), mizorbine, methotrexate, dexamethasone, ISAtx-247, SDZ ASM 981 (pimecrolimus, Elidel), CTLA41g (Abatacept), belatacept, LFA31g, etanercept (sold as Enbrel by Immunex), adalimumab (Humira), infliximab (Remicade), an anti-LFA-1 antibody, natalizumab (Antegren), Enlimomab, gavilimomab, antithymocyte immunoglobulin, siplizumab, Alefacept efalizumab, pentasa, mesalazine, asacol, codeine phosphate, benorylate, fenbufen, naprosyn, diclofenac, etodolac and indomethacin, aspirin and ibuprofen.

In certain embodiments, a pharmaceutical combination or composition described herein is administered to the subject prior to treatment with another chemotherapeutic agent, during treatment with another chemotherapeutic agent, after administration of another chemotherapeutic agent, or a combination thereof.

In some embodiments, the selective pharmaceutical combination or composition can be administered to the subject such that the other chemotherapeutic agent can be administered either at higher doses (increased chemotherapeutic dose intensity) or more frequently (increased chemotherapeutic dose density). Dose-dense chemotherapy is a chemotherapy treatment plan in which drugs are given with less time between treatments than in a standard chemotherapy treatment plan. Chemotherapy dose intensity represents unit dose of chemotherapy administered per unit time. Dose intensity can be increased or decreased through altering dose administered, time interval of administration, or both.

In one embodiment of the invention, the pharmaceutical combination or composition described herein can be administered in a concerted regimen with another agent such as a non-DNA-damaging, targeted anti-neoplastic agent or a hematopoietic growth factor agent. It has recently been reported that the untimely administration of hematopoietic growth factors can have serious side effects. For example, the use of the EPO family of growth factors has been associated with arterial hypertension, cerebral convulsions, hypertensive encephalopathy, thromboembolism, iron deficiency, influenza like syndromes and venous thrombosis. The G-CSF family of growth factors has been associated with spleen enlargement and rupture, respiratory distress syndrome, allergic reactions and sickle cell complications. By combining the administration of the pharmaceutical combination or composition as described herein with the timely administration of hematopoietic growth factors, for example, at the time point wherein the affected cells are no longer under growth arrest, it is possible for the health care practitioner to decrease the amount of the growth factor to minimize the unwanted adverse effects while achieving the desired therapeutic benefit. As such, in one embodiment, the use of the pharmaceutical combination, composition, or methods described herein is combined with the use of hematopoietic growth factors including, but not limited to, granulocyte colony stimulating factor (G-CSF, for example, sold as Neupogen (filgrastin), Neulasta (peg-filgrastin), or lenograstin), granulocyte-macrophage colony stimulating factor (GM-CSF, for example sold as molgramostim and sargramostim (Leukine)), M-CSF (macrophage colony stimulating factor), thrombopoietin (megakaryocyte growth development factor (MGDF), for example sold as Romiplostim and Eltrombopag) interleukin (IL)-12, interleukin-3, interleukin-11 (adipogenesis inhibiting factor or oprelvekin), SCF (stem cell factor, steel factor, kit-ligand, or KL) and erythropoietin (EPO), and their derivatives (sold as for example epoetin-α as Darbopoetin, Epocept, Nanokine, Epofit, Epogin, Eprex and Procrit; epoetin-(3 sold as for example NeoRecormon, Recormon and Micera), epoetin-delta (sold as for example Dynepo), epoetin-omega (sold as for example Epomax), epoetin zeta (sold as for example Silapo and Reacrit) as well as for example Epocept, EPOTrust, Erypro Safe, Repoeitin, Vintor, Epofit, Erykine, Wepox, Espogen, Relipoeitin, Shanpoietin, Zyrop and EPIAO). In one embodiment, the pharmaceutical combination or composition is administered prior to administration of the hematopoietic growth factor. In one embodiment, the hematopoietic growth factor administration is timed so that the pharmaceutical combination or composition's effect on HSPCs has dissipated. In one embodiment, the growth factor is administered at least 20 hours after the administration of a pharmaceutical combination or composition described herein.

If desired, multiple doses of a pharmaceutical combination or composition described herein can be administered to the subject. Alternatively, the subject can be given a single dose of a pharmaceutical combination or composition described herein.

In one embodiment, the activity of an active compound for a purpose described herein can be augmented through conjugation to an agent that targets the diseased or abnormally proliferating cell or otherwise enhances activity, delivery, pharmacokinetics or other beneficial property.

A selected compound described herein can be administered in conjugation or combination with a Fv fragment. Fv fragments are the smallest fragment made from enzymatic cleavage of IgG and IgM class antibodies. Fv fragments have the antigen-binding site made of the VH and VC regions, but they lack the CH1 and CL regions. The VH and VL chains are held together in Fv fragments by non-covalent interactions.

In one embodiment, a selected compound as described herein can be administered in combination with an antibody fragment selected from the group consisting of an ScFv, domain antibody, diabody, triabody, tetrabody, Bis-scFv, minibody, Fab2, or Fab3 antibody fragment. In one embodiment, the antibody fragment is a ScFv. Genetic engineering methods allow the production of single chain variable fragments (ScFv), which are Fv type fragments that include the VH and VL domains linked with a flexible peptide When the linker is at least 12 residues long, the ScFv fragments are primarily monomeric. Manipulation of the orientation of the V-domains and the linker length creates different forms of Fv molecules linkers that are 3-11 residues long yield scFv molecules that are unable to fold into a functional Fv domain. These molecules can associate with a second scFv molecule, to create a bivalent diabody. In one embodiment, the antibody fragment administered in combination with a selected compound described herein is a bivalent diabody. If the linker length is less than three residues, scFv molecules associate into triabodies or tetrabodies. In one embodiment, the antibody fragment is a triabody. In one embodiment, the antibody fragment is a tetrabody. Multivalent scFvs possess greater functional binding affinity to their target antigens than their monovalent counterparts by having binding to two more target antigens, which reduces the off-rate of the antibody fragment. In one embodiment, the antibody fragment is a minibody. Minibodies are scFv-CH3 fusion proteins that assemble into bivalent dimers. In one embodiment, the antibody fragment is a Bis-scFv fragment. Bis-scFv fragments are bispecific. Miniaturized ScFv fragments can be generated that have two different variable domains, allowing these Bis-scFv molecules to concurrently bind to two different epitopes.

In one embodiment, a selected compound described herein is administered in conjugation or combination with a bispecific dimer (Fab2) or trispecific dimer (Fab3). Genetic methods are also used to create bispecific Fab dimers (Fab2) and trispecific Fab trimers (Fab3). These antibody fragments are able to bind 2 (Fab2) or 3 (Fab3) different antigens at once.

In one embodiment, a selected compound described herein is administered in conjugation or combination with an rIgG antibody fragment. rIgG antibody fragments refers to reduced IgG (75,000 daltons) or half-IgG. It is the product of selectively reducing just the hinge-region disulfide bonds. Although several disulfide bonds occur in IgG, those in the hinge-region are most accessible and easiest to reduce, especially with mild reducing agents like 2-mercaptoethylamine (2-MEA). Half-IgG are frequently prepared for the purpose of targeting the exposing hinge-region sulfhydryl groups that can be targeted for conjugation, either antibody immobilization or enzyme labeling.

In other embodiments, a selected active compound described herein can be linked to a radioisotope to increase efficacy, using methods well known in the art. Any radioisotope that is useful against cancer cells can be incorporated into the conjugate, for example, but not limited to, ¹³¹I, ¹²³I, ¹⁹²Ir, ³²P, ⁹⁰Sr, ¹⁹⁸Au, ²²⁶Ra, ⁹⁰Y, ²⁴¹Am, ²⁵²Cf, ⁶⁰Co, or ¹³⁷Cs.

Examples of early and recent antibody-drug conjugates, discussing drugs, linker chemistries and classes of targets for product development that may be used in the present invention can be found in the reviews by Casi, G. and Neri, D., Antibody-drug conjugates: basic concepts, examples and future perspectives, J. Control Release 161(2):422-428, 2012, Chari, R. V., Targeted cancer therapy: conferring specificity to cytotoxic drugs, Acc. Chem. Rev., 41(1):98-107, 2008, Sapra, P. and Shor, B., Monoclonal antibody-based therapies in cancer: advances and challenges, Pharmacol. Ther., 138(3):452-69, 2013, Schliemann, C. and Neri, D., Antibody-based targeting of the tumor vasculature, Biochim. Biophys. Acta., 1776(2):175-92, 2007, Sun, Y., Yu, F., and Sun, B. W., Antibody-drug conjugates as targeted cancer therapeutics, Yao Xue Xue Bao, 44(9):943-52, 2009, Teicher, B. A., and Chari, R. V., Antibody conjugate therapeutics: challenges and potential, Clin. Cancer Res., 17(20):6389-97, 2011, Firer, M. A., and Gellerman, G. J., Targeted drug delivery for cancer therapy: the other side of antibodies, J. Hematol. Oncol., 5:70, 2012, Vlachakis, D. and Kossida, S., Antibody Drug Conjugate bioinformatics: drug delivery through the letterbox, Comput. Math. Methods Med., 2013; 2013:282398, Epub 2013 Jun. 19, Lambert, J. M., Drug-conjugated antibodies for the treatment of cancer, Br. J. Clin. Pharmacol., 76(2):248-62, 2013, Concalves, A., Tredan, O., Villanueva, C. and Dumontet, C., Antibody-drug conjugates in oncology: from the concept to trastuzumab emtansine (T-DM1), Bull. Cancer, 99(12):1183-1191, 2012, Newland, A. M., Brentuximab vedotin: a CD-30-directed antibody-cytotoxic drug conjugate, Pharmacotherapy, 33(1):93-104, 2013, Lopus, M., Antibody-DM1 conjugates as cancer therapeutics, Cancer Lett., 307(2):113-118, 2011, Chu, Y. W. and Poison, A., Antibody-drug conjugates for the treatment of B-cell non-Hodgkin's lymphoma and leukemia, Future Oncol., 9(3):355-368, 2013, Bertholjotti, I., Antibody-drug conjugate a new age for personalized cancer treatment, Chimia, 65(9): 746-748, 2011, Vincent, K. J., and Zurini, M., Current strategies in antibody engineering: Fc engineering and pH-dependent antigen binding, bispecific antibodies and antibody drug conjugates, Biotechnol. J., 7(12):1444-1450, 2012, Haeuw, J. F., Caussanel, V., and Beck, A., Immunoconjugates, drug-armed antibodies to fight against cancer, Med. Sci., 25(12):1046-1052, 2009 and Govindan, S. V., and Goldenberg, D. M., Designing immunoconjugates for cancer therapy, Expert Opin. Biol. Ther., 12(7):873-890, 2012.

In one embodiment the pharmaceutical composition or combination as described herein can be used to treat any disorder described herein.

### Synthesis of the Compounds of the Application

Compounds of the invention can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999, incorporated by reference herein, are useful and recognized reference textbooks of organic synthesis known to those in the art. The following descriptions of synthetic methods are designed to illustrate, but not to limit, general procedures for the preparation of compounds of the invention.

The compounds of disclosed herein may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of disclosed herein.

Those skilled in the art will recognize if a stereocenter exists in the compounds of disclosed herein. Accordingly, the invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

All the abbreviations used in this application are found in “Protective Groups in Organic Synthesis” by John Wiley & Sons, Inc, or the MERCK INDEX by MERCK & Co., Inc, or other chemistry books or chemicals catalogs by chemicals vendor such as Aldrich, or according to usage know in the art.

Exemplary synthetic schemes for preparing the bifunctional compounds of the invention are shown in below.

All reactions can be monitored with standard methods and procedures either known to those skilled in the art, or which will be apparent to the skilled artisan in light of the teachings herein. In one embodiment, the reactions are monitored with Waters Acquity UPLC/MS system (Waters PDA eX Detector, QDa Detector, Sample manager-FL, Binary Sovent Manager) using Acquity UPLC® BEH C18 column (2.1×50 mm, 1.7 m particle size): solvent gradient=80% A at 0 min, 5% A at 2 min; solvent A=0.1% formic acid in Water; solvent B=0.1% formic acid in Acetonitrile; flow rate: 0.6 mL/min (method A), or Analytical HPLC was carried out on YMC-Park Pro C18, 150×4.6 mm column using gradient condition (5-100% B over 7 min, flow rate=1.0 mL/min) (method B). Reaction products were purified by flash column chromatography using CombiFlash®Rf with Teledyne Isco RediSepRf High Performance Gold or Silicycle SiliaSep™ High Performance columns (4 g, 12 g, 24 g, 40 g, or 80 g) and Waters HPLC system using SunFire™ Prep C18 column (19×100 mm, 5 μm particle size): solvent gradient=80% A at 0 min, 5% A at 25 min; solvent A=0.035% TFA in Water; solvent B=0.035% TFA in MeOH; flow rate: 25 mL/min. The purity of all compounds was over 95% and was analyzed with Waters LC/MS system. ¹H NMR was obtained using a 600 MHz Varian Inova-600, 500 MHz Bruker Avance III or 400 MHz Brucker Avance. Chemical shifts are reported relative to methanol (δ=3.31) or dimethyl sulfoxide (δ=2.50) for ¹H NMR. Data are reported as (br=broad, s=singlet, d=doublet, t=triplet, q=quartet, m=multiplet).

### Biological Assays

### Lantha Screening

Lantha screening is performed by following the method reported in Nature Chemical Biology, 10, 1006-1012 (2014).

### Immunoblotting

Cells are seeded at the desired density the day before treatment starts with bifunctional compounds of the application at various concentration. After 4 to 12 hrs, cells are washed with buffer and lysed. The lysates are centrifuged and the supernatant is collected. Protein concentrations are measured using a protein assay kit, such as the BCA protein assay kit, Pierce, catalog number 23225) and normalized. Samples are run on a SDS-PAGE gel, and transferred to a PVDF membrane. The PVDF membrane is probed with the appropriate antibody.

### Anti-Proliferation Assay

Cells are seeded and incubated for 3 d after bifunctional compounds of the application are added. Cell viability is measured via MTS Assay. This assay uses a colorimetric method to determine the number of viable cells based on the bioreduction of MTS by cells to a formazan product that is soluble in cell culture medium and can be detected spectrophotometrically. In a typical experiment, the supernatant is removed and replaced by 100 μl of RPMI media supplemented with MTS reagent and PMS. The plates are measured with Perkin Elmer EnVision after reaching an optical density (OD) of 1.0-2.0 at a wavelength of 490 nm. The cell numbers are normalized compared to DMSO control, and the EC₅₀ values are calculated using GraphPad Prism.

### EXAMPLES

### Example 1: Synthesis of Compound PP1

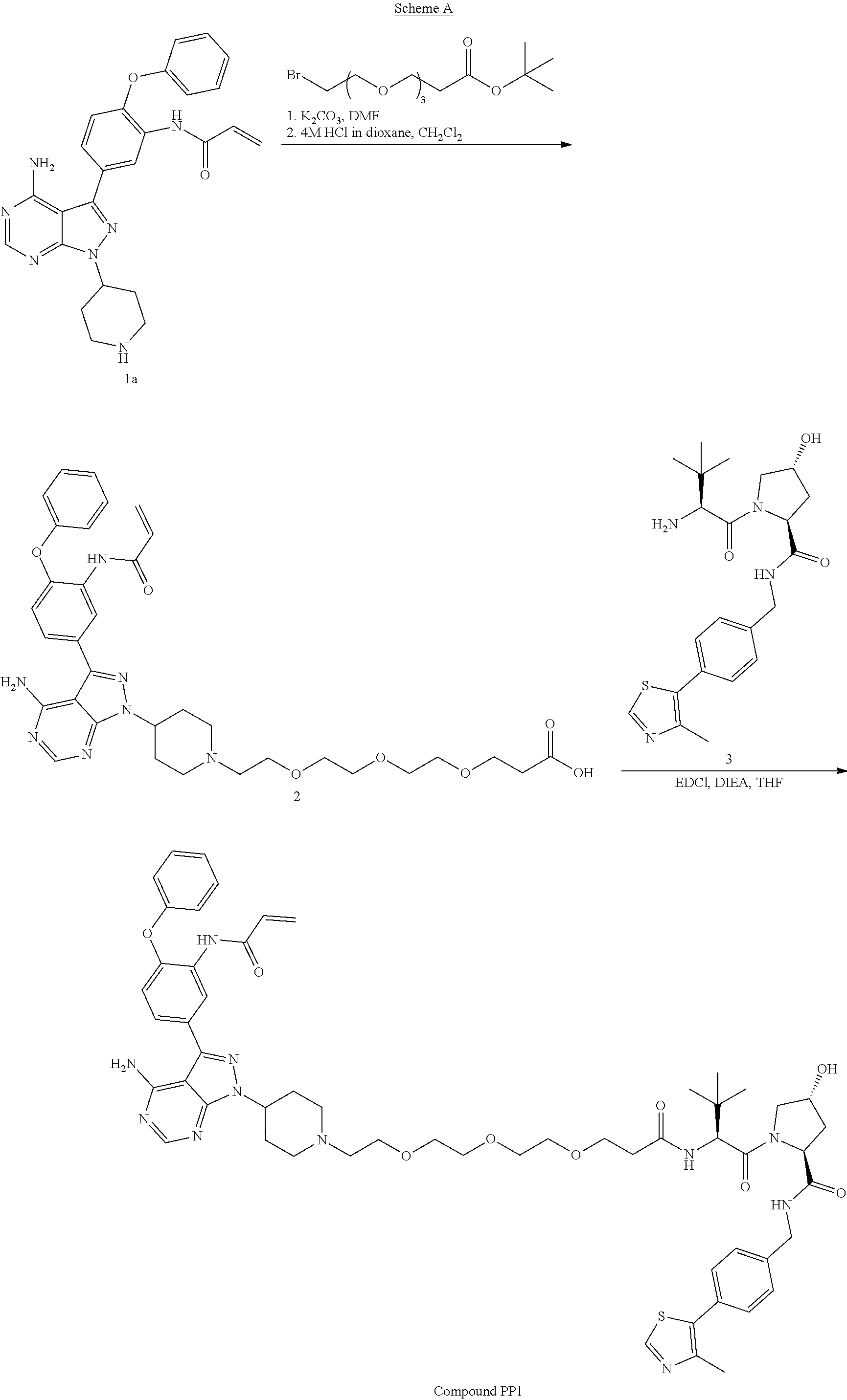

Compound PP1 was prepared according to Synthetic Scheme A. Compound 1a was synthesized with analogous procedures to those described in Bioorganic & Medicinal Chemistry Letters, 25(16), 3382-3389; 2015.

### Step 1: Compound 3

Compound 3 was prepared by following the procedures reported in Journal of Medicinal Chemistry, 57, 8657-8663 (2014).

### Step 2: Compound 2

To a solution of Compound 1 (100 mg, 0.220 mmol) and tert-butyl 3-(2-(2-(2-bromoethoxy)ethoxy)ethoxy)propanoate (75 mg, 1.38 mmol) in N,N-dimehtylformamide (2 mL) was added potassium carbonate (61 mg, 0.440 mmol). Following stirring for 5 hours, the reaction mixture was cooled to 0° C. and diluted with EtOAc and water. The resulting mixture was washed with water five times and dried over sodium sulfate, filtered and concentrated under reduced pressure. The resulting residue was purified by flash column chromatography (1:99 to 50:50, EtOAc/CH₂Cl₂) to afford tert-butyl 3-(2-(2-(2-(4-(3-(3-acrylamido-4-phenoxyphenyl)-4-amino-1H-pyrazolo[3,4-d]pyrimidin-1-yl)piperidin-1-yl)ethoxy)ethoxy)ethoxy)propanoate (124 mg, 79%).

To a solution of tert-butyl 3-(2-(2-(2-(4-(3-(3-acrylamido-4-phenoxyphenyl)-4-amino-1H-pyrazolo[3,4-d]pyrimidin-1-yl)piperidin-1-yl)ethoxy)ethoxy)ethoxy)propanoate (50 mg, 0.070 mmol) in CH₂Cl₂ (0.5 mL) was added 4 M HCl solution in dioxane (1 mL). After stirring for 2 hours, the reaction mixture was concentrated under reduced pressure. The residue was carried forward in the next step without further purification.

### Step 3: Compound PP1

To a solution of 3-(2-(2-(2-(4-(3-(3-acrylamido-4-phenoxyphenyl)-4-amino-1H-pyrazolo[3,4-d]pyrimidin-1-yl)piperidin-1-yl)ethoxy)ethoxy)ethoxy)propanoic acid (26 mg, 0.039 mmol) and Compound 3 (17 mg, 0.039 mmol) in THF (1 mL) were added EDCI (15 mg, 0.079 mmol) and DIEA (21.0 μL, 0.118 mmol). After stirring for 6 hours, the reaction mixture was diluted with EtOAc and washed with water. The organic layer was dried over sodium sulfate, filtered, concentrated under reduced pressure and purified by preparative high performance liquid chromatography (HPCL) to obtain Compound PP1 (14 mg, 34%) as an off-white solid. MS m/z: 1072.75 [M+1]⁺; ¹H NMR 600 MHz (DMSO-d₆) δ 9.99 (s, 1H), 9.66 (br, 2H), 8.96 (s, 1H), 8.55 (t, J=5.9 Hz, 1H), 8.42 (s, 1H), 8.27 (s, 1H), 7.89 (d, J=9.4 Hz, 1H), 7.44-7.32 (m, 7H), 7.17 (t, J=7.0 Hz, 1H), 7.11 (d, J=7.6 Hz, 2H), 7.01 (d, J=8.2 Hz, 1H), 6.69 (dd, J=17.0, 10.6 Hz, 1H), 6.25 (dd, J=17.0, 1.7 Hz, 1H), 5.74 (dd, J=10.6, 1.8 Hz, 1H), 5.09 (br, 1H), 5.06-4.98 (m, 1H), 4.52 (d, J=9.4 Hz, 1H), 4.44-4.35 (m, 2H), 4.33 (br, 1H), 4.20 (dd, J=15.9, 5.3 Hz, 1H), 3.81-3.73 (m, 2H), 3.71-3.36 (m, 14H), 3.34-3.20 (m, 4H), 2.54-2.45 (m, 2H), 2.42 (s, 3H), 2.36-2.29 (m, 2H), 2.20-2.15 (m, 2H), 2.05-1.98 (m, 1H), 1.91-1.84 (m, 1H), 0.89 (s, 9H).

### Example 2: Synthesis of Compound PP2 and Compound PP8

Compound PP2 and Compound PP8 were synthesized by following the procedures analogous to the synthesis of Compound PP1 as described above and shown in Synthetic Scheme A.

Compound PP2: MS m/z: 926.75 [M+1]⁺; ¹H NMR 600 MHz (DMSO-d₆) δ 10.00 (s, 1H), 9.98 (br, 2H), 8.98 (s, 1H), 8.79 (d, J=8.8 Hz, 1H), 8.58 (t, J=5.9 Hz, 1H), 8.42 (s, 1H), 8.32 (s, 1H), 7.48-7.34 (m, 7H), 7.19 (t, J=7.0 Hz, 1H), 7.12 (d, J=7.6 Hz, 2H), 7.02 (d, J=8.2 Hz, 1H), 6.69 (dd, J=17.0, 10.6 Hz, 1H), 6.25 (dd, J=17.0, 1.8 Hz, 1H), 5.75 (dd, J=10.1, 1.8 Hz, 1H), 5.12 (br, 1H), 5.08-5.00 (m, 1H), 4.60 (d, J=9.4 Hz, 1H), 4.48-4.39 (m, 3H), 4.36 (br, 1H), 4.26-4.17 (m, 2H), 4.14-4.03 (m, 2H), 3.72-3.66 (m, 1H), 3.65-3.56 (m, 3H), 3.41-3.30 (m, 2H), 2.61-2.50 (m, 2H), 2.43 (s, 3H), 2.21-2.12 (m, 2H), 2.08-2.01 (m, 1H), 1.94-1.87 (m, 1H), 0.97 (s, 9H).

Compound PP8: MS m/z: 1049.46 [M+1]⁺; ¹H NMR (500 MHz, DMSO-d₆) δ 9.54 (s, 1H), 8.98 (s, 1H), 8.56 (t, J=6.1 Hz, 1H), 8.36-8.25 (m, 2H), 7.95-7.88 (m, 2H), 7.47 (t, J=8.0 Hz, 2H), 7.39 (dd, J=18.4, 8.3 Hz, 4H), 7.30 (d, J=8.6 Hz, 1H), 7.28-7.22 (m, 1H), 7.18 (d, J=7.8 Hz, 2H), 5.12-5.00 (m, 1H), 4.54 (d, J=9.4 Hz, 1H), 4.47-4.30 (m, 4H), 4.22 (dd, J=15.8, 5.4 Hz, 2H), 3.80-3.76 (m, 2H), 3.72-3.49 (m, 15H), 3.36-3.26 (m, 4H), 2.44 (s, 3H), 2.37-2.31 (m, 1H), 2.23-2.16 (m, 2H), 2.07-2.00 (m, 1H), 1.92-1.86 (m, 1H), 0.91 (s, 9H).

### Example 3: Synthesis of Compound PP3

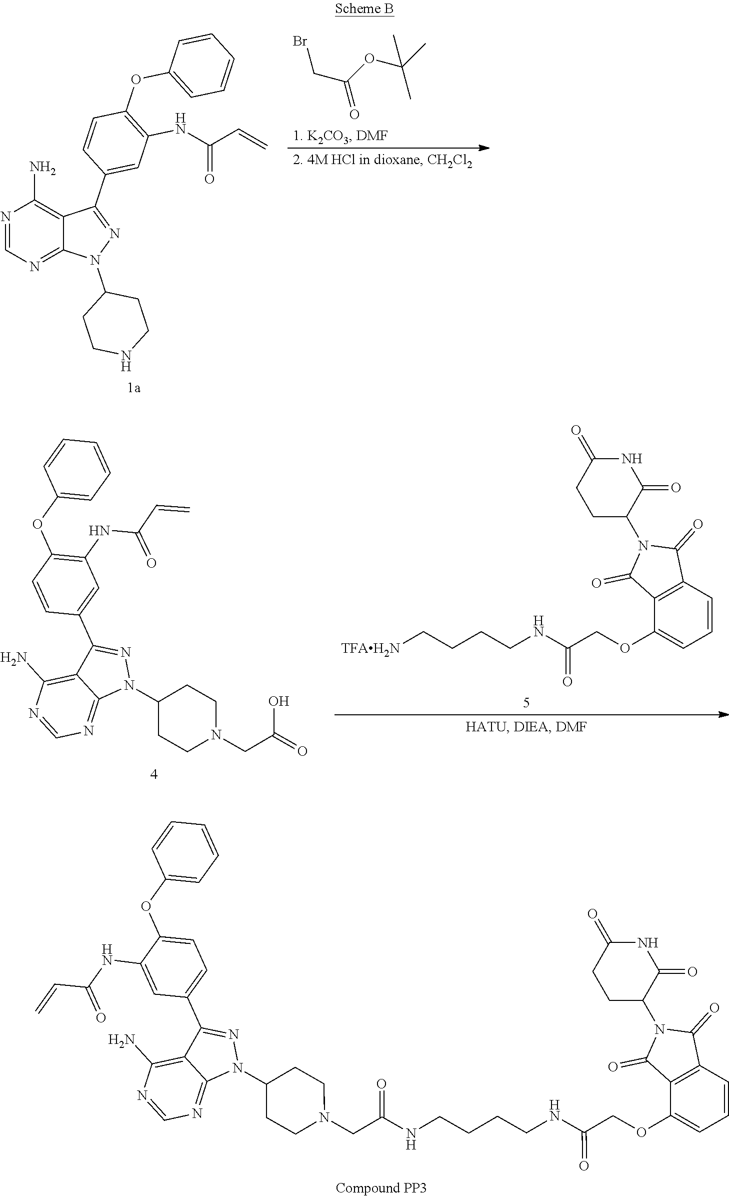

Compound PP3 was prepared according to Synthetic Scheme B. Compound 1a was synthesized with analogous procedures to those described in Bioorganic & Medicinal Chemistry Letters, 25(16), 3382-3389; 2015.

### Step 1: Compound 4

Compound 4 was synthesized following the same procedure as Compound 2 as described above and shown in Synthetic Scheme A.

### Step 2: Compound 5

Compound 5 was prepared by following the procedures reported in Nature, 512, 49-53 (2014).

### Step 3: Compound PP3

A 0.1 M solution of N-(4-aminobutyl)-2-((2-(2,6-dioxopiperidin-3-yl)-1,3-dioxoisoindolin-4-yl)oxy)acetamide trifluoroacetate in DMF (242 μL, 0.0242 mmol) was added to 2-(4-(3-(3-acrylamido-4-phenoxyphenyl)-4-amino-1H-pyrazolo[3,4-d]pyrimidin-1-yl)piperidin-1-yl)acetic acid (12.5 mg, 0.0242 mmol). DIPEA (12.6 μL, 0.0726 mmol) and HATU (9.2 mg, 0.0242 mmol) were added and the mixture was stirred for 23 hours at room temperature. The mixture was diluted with methanol and purified by preparative HPLC to afford an off-white solid (3.14 mg, 0.00310 mmol, 13%). MS m/z: 898.45 [M+H]⁺; ¹H NMR (400 MHz, Methanol-d₄) δ 8.41 (s, 1H), 8.36 (s, 1H), 8.16-8.11 (m, 1H), 7.86-7.76 (m, 2H), 7.56-7.47 (m, 2H), 7.45-7.40 (m, 2H), 7.22 (d, J=7.5 Hz, 1H), 7.14-7.11 (m, 1H), 7.05 (d, J=8.5 Hz, 1H), 6.58 (dd, J=16.9, 10.1 Hz, 1H), 6.39 (d, J=17.1 Hz, 1H), 5.80 (d, J=11.9 Hz, 1H), 5.16-5.11 (m, 1H), 4.77 (s, 2H), 4.00-3.96 (m, 1H), 3.82-3.74 (m, 2H), 3.35 (s, 2H), 3.00-2.95 (m, 1H), 2.89-2.81 (m, 2H), 2.80-2.66 (m, 4H), 2.40-2.29 (m, 2H), 2.19-2.09 (m, 2H), 1.68-1.55 (m, 4H), 1.37 (dd, J=6.8, 3.4 Hz, 1H), 1.31-1.27 (m, 1H), 1.23 (d, J=6.7 Hz, 1H).

### Example 4: Synthesis of Compound PP4 and Compound PP5

Compound PP4 and Compound PP5 were synthesized by following the procedures analogous to the synthesis of Compound PP3 as described above and shown in Synthetic Scheme B.

Compound PP4: MS m/z: 954.57 [M+H]⁺; ¹H NMR (400 MHz, Methanol-d₄) δ 8.37 (d, J=17.4 Hz, 2H), 8.07-7.97 (m, 1H), 7.86-7.71 (m, 2H), 7.62-7.48 (m, 2H), 7.47-7.36 (m, 2H), 7.21 (d, J=7.6 Hz, 1H), 7.12 (d, J=7.9 Hz, 1H), 7.04 (d, J=8.6 Hz, 1H), 6.55 (d, J=10.2 Hz, 1H), 6.38 (d, J=15.4 Hz, 1H), 5.79 (d, J=12.1 Hz, 1H), 5.15-5.10 (m, 1H), 4.75 (d, J=3.1 Hz, 2H), 4.04-3.97 (m, 1H), 3.87-3.72 (m, 2H), 3.35-3.32 (m, 1H), 3.26 (s, 1H), 3.22-3.10 (m, 2H), 3.01-2.64 (m, 10H), 2.40-2.29 (m, 1H), 2.17-2.11 (m, 1H), 1.66-1.50 (m, 4H), 1.38-1.26 (m, 8H).

Compound PP5: MS m/z: 1030.64 [M+H]⁺; ¹H NMR (400 MHz, Methanol-d₄) δ 8.35 (d, J=36.6 Hz, 2H), 8.10-8.01 (m, 1H), 7.87-7.71 (m, 2H), 7.54 (dd, J=16.5, 7.5 Hz, 2H), 7.48-7.37 (m, 2H), 7.20 (s, 1H), 7.12 (d, J=7.6 Hz, 1H), 7.03 (d, J=8.3 Hz, 1H), 6.54 (s, 1H), 6.40 (s, 1H), 5.79 (d, J=11.7 Hz, 1H), 5.11 (s, 1H), 4.76 (d, J=7.3 Hz, 2H), 3.69-3.32 (m, 16H), 3.14-2.58 (m, 10H), 2.16 (s, 1H), 1.91-1.72 (m, 4H).

### Example 5: Synthesis of Compound PP6

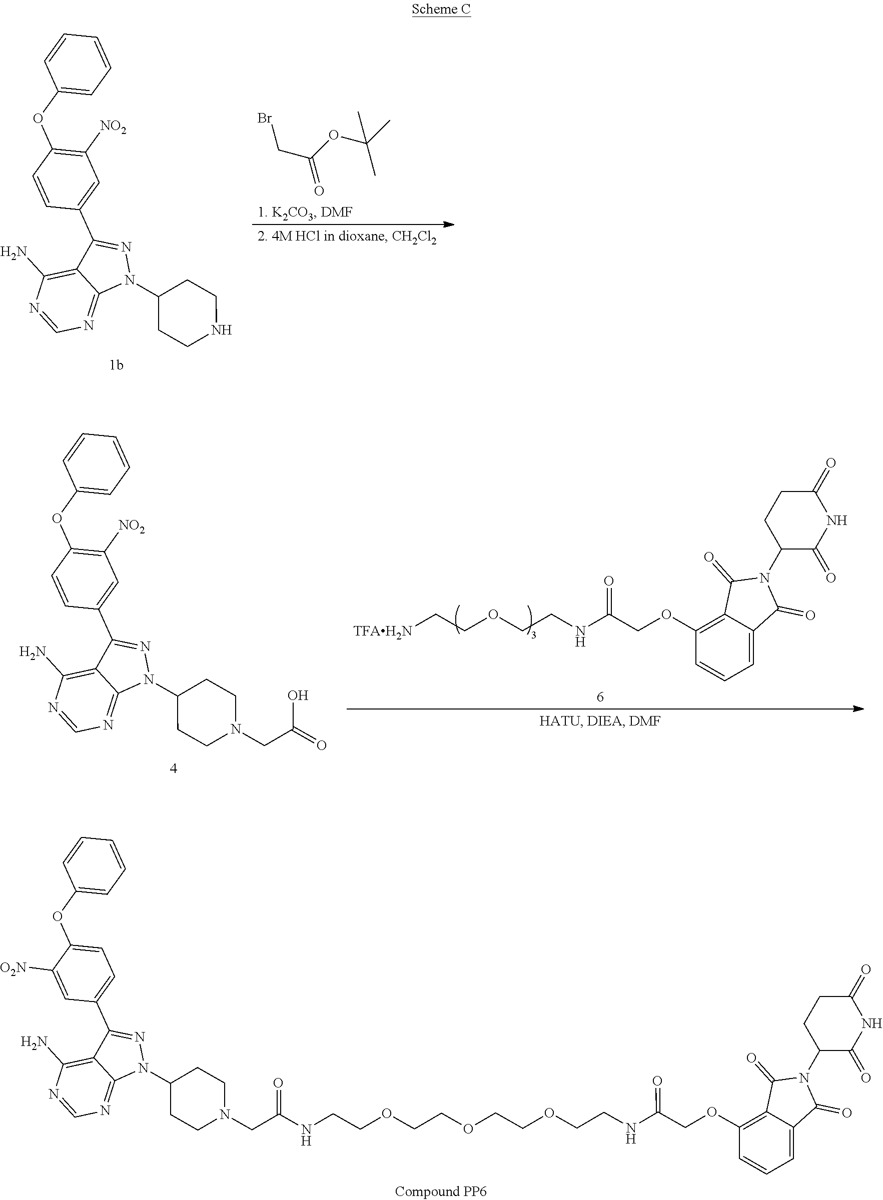

Compound PP6 was prepared according to Synthetic Scheme C. The synthesis of Compound 1b is described in Bioorganic & Medicinal Chemistry Letters, 25(16), 3382-3389; 2015.

### Step 1: Compound 4

Compound 4 was synthesized following the same procedure of Compound 2 as described above and shown in Synthetic Scheme A.

### Step 2: Compound 6

Compound 6 was prepared by following the procedures reported in Nature, 512, 49-53 (2014).

### Step 3: Compound PP6

To a solution of Compound 4 (123 mg, 0.250 mmol) and Compound 6 (156 mg, 0.250 mmol) in DMF (1.5 mL) were added HATU (190 mg, 0.500 mmol) and DIEA (0.26 mL, 1.5 mmol) and the mixture was stirred for 5 hours. The resulting mixture was diluted with DMSO and purified by HPLC to afford Compound PP6 as a yellow solid (32 mg, 13%). MS m/z: 978.97 [M+1]⁺; ¹HNMR (500 MHz, DMSO-d₆) δ 11.11 (s, 1H), 10.01 (s, 1H), 8.68 (s, 1H), 8.34 (s, 1H), 8.31-8.25 (m, 1H), 8.00 (s, 1H), 7.93 (d, J=8.8 Hz, 1H), 7.81 (t, J=7.9 Hz, 1H), 7.48 (dd, J=14.9, 7.4 Hz, 4H), 7.40 (d, J=8.5 Hz, 1H), 7.30 (d, J=8.6 Hz, 1H), 7.25 (t, J=7.4 Hz, 1H), 7.18 (d, J=7.9 Hz, 2H), 5.17-4.99 (m, 2H), 4.78 (s, 2H), 3.96 (s, 2H), 3.63 (d, J=11.2 Hz, 3H), 3.46 (t, J=4.9 Hz, 5H), 3.41-3.27 (m, 6H), 2.99-2.81 (m, 2H), 2.68-2.53 (m, 3H), 2.25-2.12 (m, 2H), 2.10-1.98 (m, 1H).

### Example 6: Synthesis of Compound PP7

Compound PP7 was synthesized by following the procedures analogous to the synthesis of Compound PP6 as described above and shown in Synthetic Scheme C.

Compound PP7: MS m/z: 874.92 [M+1]⁺; ¹H NMR (500 MHz, DMSO-d₆) δ 11.12 (s, 1H), 9.98 (s, 1H), 8.55 (s, 1H), 8.35-8.22 (m, 2H), 7.99 (t, J=5.4 Hz, 1H), 7.93 (d, J=8.3 Hz, 1H), 7.82 (t, J=7.9 Hz, 1H), 7.49 (dd, J=15.6, 7.5 Hz, 3H), 7.40 (d, J=8.4 Hz, 1H), 7.30 (d, J=8.6 Hz, 1H), 7.25 (t, J=7.4 Hz, 1H), 7.18 (d, J=7.9 Hz, 2H), 5.15-5.00 (m, 2H), 4.78 (s, 2H), 3.94 (s, 2H), 3.63 (d, J=10.6 Hz, 2H), 3.39-3.29 (m, 2H), 3.17 (s, 4H), 2.95-2.84 (m, 2H), 2.65-2.54 (m, 3H), 2.18 (d, J=12.0 Hz, 2H), 2.07-2.00 (m, 1H), 1.46 (s, 4H).

### Example 7: Binding affinities of representative bifunctional compounds of the application

Binding affinities (IC₅₀) of representative compounds were measured by the Life Technologies LanthaScreen Eu kinase binding assay, which was previously described (Xie T. et al., Nat. Chem. Biol. 2014, 10, 1006-1012). The results are shown in Table 1.

TABLE 1
Binding Affinities of Compounds PP6,
PP7, and PP8

| | |
|---|---|
| Compound PP6 | B |
| Compound PP7 | B |
| Compound PP8 | C |

A: IC₅₀ < 10 nM;
B: 10 nM < IC₅₀ < 100 nM;
C: IC₅₀ > 100 nM.

### Example 8: Antiproliferation Activities of Representative Bifunctional Compounds of the Application

Five cell lines, PC9-GR4, 826-GR6, Ovacar 8, A549, and Ovacar 5 were grown and treated with representative compounds Viability of the cells after the treatment was assessed by MTS assay. The results are shown in Table 2.

TABLE 2
The Effect of Compounds PP2, PP8, and PP4 on Various Cell Lines

| | Cell lines (EC₅₀, μM) | | | | |
|---|---|---|---|---|---|
| | PC9-GR4 | 826-GR6 | Ovacar 8 | A549 | Ovacar 5 |
| Compound PP2 | B | B | A | B | B |
| Compound PP8 | C | — | — | — | — |
| Compound PP4 | — | — | B | — | — |

A: EC₅₀ < 5M;
B: 5 μM < EC₅₀ < 15 μM;
C: EC₅₀ > 15 μM.

### Example 9: Effect of Her3 Degradation

Her3 protein degradation was assessed by Western blots after treatment of PC9-GR4 cell lines or Ovacar 8 cell lines with 2 μM of representative compounds for 4 hour and 8 hour. The results are shown in Table 3.

TABLE 3
Her3 Degradation of Representative Compounds

| | |
|---|---|
| Compound PP2 | + |
| Compound PP1 | − |
| Compound PP7 | − |
| Compound PP6 | − |
| Compound PP8 | + |
| Compound PP3 | + |
| Compound PP4 | + |
| Compound PP5 | − |

(+): Her3 protein was degraded;
(−): Her3 protein was not degraded.

### Example 10: Biological assays

### Lantha Screening

Lantha screening was performed by following the method reported in Nature Chemical Biology, 10, 1006-1012 (2014).

### Immunoblotting

Cells were seeded at a density of 4×10\hat{ } per 6 cm plate the day before treatment started with representative compounds of the application at the indicated concentration. After 4 to 12 hours, cells were washed with phosphate-buffered saline. Lysis buffer included 50 mM Tris-HCl, 150 mM NaCl, 1% NP-40, and 5 mM EDTA, pH 7.4+/−0.2, Roche PhosSTOP phosphatase inhibitor cocktail tablets and Roche Complete Protease inhibitor cocktail tablets. Cell lysis was accomplished by the addition of lysis buffer for 5-10 minutes on ice. Lysates were centrifuged in a microcentrifuge at 14,000 r.p.m. for 15 minutes at 4° C. and the supernatant was collected. Protein concentrations were measured using BCA protein assay kit (Pierce, catalog number 23225) and normalized. Samples were run on a 4%-12% SDS-PAGE gel at 120 V. After transfer, the PVDF membrane was probed with anti-Her3 antibody, Santa Cruz, catalog number sc-285 at 1:1000 dilution.

### Anti-Proliferation Assay

The anti-proliferation assay was carried out using 96-well clear bottom plates. 1,000-2000 cells were seeded per well with a final volume of 100 μl and incubated for 3 days after adding and titrating the indicated concentration of representative compounds of the application. Cell viability was measured via MTS Assay. This assay uses a colorimetric method to determine the number of viable cells based on the bioreduction of MTS by cells to a formazan product that is soluble in cell culture medium and can be detected spectrophotometrically. In a typical experiment, the supernatant was removed and replaced by 100 l of RPMI media supplemented with MTS reagent and PMS. The plates were measured with Perkin Elmer EnVision after reaching an optical density (OD) of 1.0-2.0 at a wavelength of 490 nm. The cell numbers were normalized compared to DMSO control, and the EC₅₀ values were calculated using GraphPad Prism.

### EQUIVALENTS

Those skilled in the art will recognize, or be able to ascertain using no more than routine experimentation, many equivalents to the specific embodiments and methods described herein. Such equivalents are intended to be encompassed by the scope of the invention.

All patents, patent applications, and literature references cited herein are hereby expressly incorporated by reference.

1. A bifunctional compound of Formula: 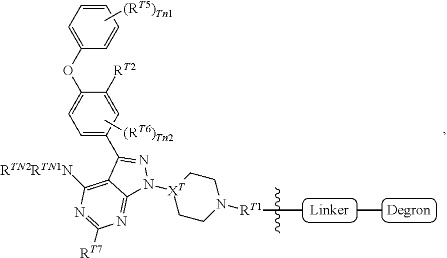
or an enantiomer, diastereomer, stereoisomer, or pharmaceutically acceptable salt thereof, wherein:
the Linker is a group that covalently binds to R^{T1} and the Degron;
the Degron is capable of binding to a ubiquitin ligase;
X^{T} is N or CH;
R^{T1} is absent, (CH₂)₀₋₃C(O)NH, or (CH₂)₀₋₃NHC(O);
R^{T2} is NO₂ or NH₂;
Tn1 is 0, 1, 2, 3, 4, or 5;
each R^{T5} is independently OH, halogen, CN, C₁-C₄ alkyl, C₁-C₄ alkyl substituted with halogen, C₁-C₄ alkoxy, or C₁-C₄ alkoxy substituted with halogen;
Tn2 is 0, 1, 2, or 3;
each R^{T6} is independently OH, halogen, CN, C₁-C₄ alkyl, C₁-C₄ alkyl substituted with halogen, C₁-C₄ alkoxy, or C₁-C₄ alkoxy substituted with halogen;
R^{T7} is H or C₁-C₄ alkyl; and
R^{TN1} and R^{TN2} are each independently H or C₁-C₄ alkyl.

2. The bifunctional compound of claim 1, wherein X^{T} is CH.

3. The bifunctional compound of claim 1, wherein R^{T1} is absent or (CH₂)₀₋₃C(O)NH.

4. The bifunctional compound of claim 1, wherein R^{T1} is (CH₂)C(O)NH.

5. The bifunctional compound of claim 1, wherein R^{T2} is NO₂.

6. The bifunctional compound of claim 1, wherein R^{T2} is NH₂.

7. The bifunctional compound of claim 1, wherein R^{T7} is H.

8. The bifunctional compound of claim 1, wherein R^{TN1} and R^{TN2} are each H.

9. The bifunctional compound of claim 1, wherein the bifunctional compound is of Formula: 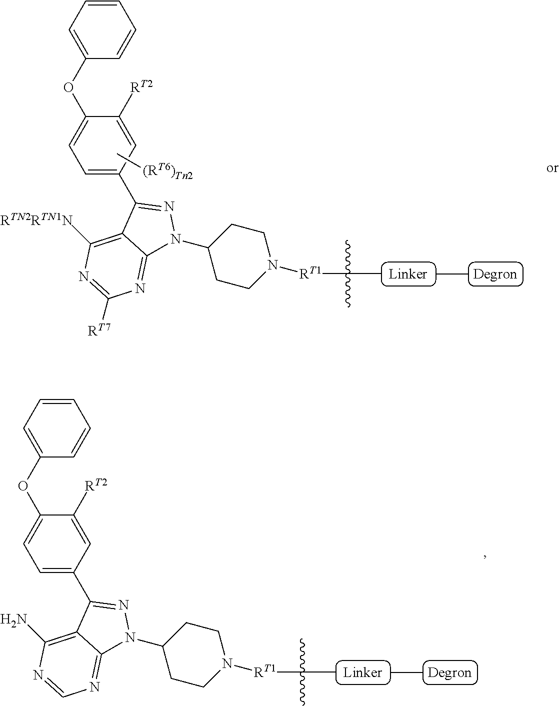
or an enantiomer, diastereomer, stereoisomer, or pharmaceutically acceptable salt thereof, wherein R^{T2} is NO₂.

10. The bifunctional compound of claim 1, wherein the Linker is of Formula L0: 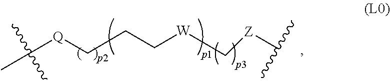
or an enantiomer, diastereomer, or stereoisomer thereof, wherein
p1 is an integer selected from 0 to 12;
p2 is an integer selected from 0 to 12;
p3 is an integer selected from 1 to 6;
each W is independently absent, CH₂, O, S, NH, or NR⁸;
Z is absent, CH₂, O, NH, or NR⁸;
each R⁸ is independently C₁-C₃ alkyl; and
Q is absent or CH₂C(O)NH,
wherein the Linker is covalently bonded to the Degron via the 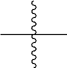
next to Q.

11. The bifunctional compound of claim 10, wherein the Linker is selected from:

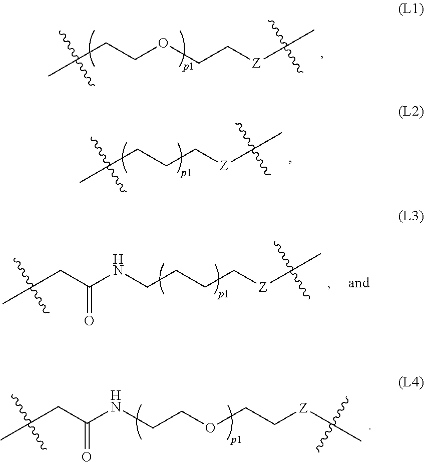

12. The bifunctional compound of claim 1, wherein the Linker is of Formula L5: 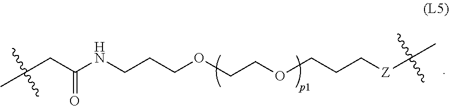
or an enantiomer, diastereomer, or stereoisomer thereof, wherein
p1 is an integer selected from 0 to 12;
Z is absent, CH₂, O, NH, or NR⁸; and
each R⁸ is independently C₁-C₃ alkyl;
wherein the Linker is covalently bonded to the Degron via the 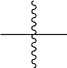
next to Q.

13. The bifunctional compound of claim 1, wherein the Degron bonds to cereblon or VHL.

14. (canceled)

15. The bifunctional compound of claim 1, wherein the Degron is of Formula D1: 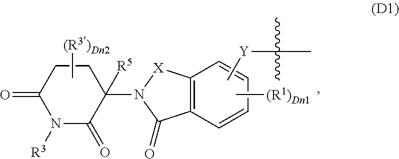
or an enantiomer, diastereomer, or stereoisomer thereof, wherein:
Y is a bond, (CH₂)₁₋₆, (CH₂)₀₋₆—O, (CH₂)₀₋₆—C(O)NR^{2′}, (CH₂)₀₋₆—NR^{2′}C(O), (CH₂)₀₋₆—NH, or (CH₂)₀₋₆—NR²;
X is C(O) or C(R³)₂;
each R¹ is independently halogen, OH, C₁-C₆ alkyl, or C₁-C₆ alkoxy;
R² is C₁-C₆ alkyl or C(O)—C₁-C₆ alkyl;
R^{2′} is H or C₁-C₆ alkyl;
each R³ is independently H or C₁-C₃ alkyl;
each R^{3′} is independently C₁-C₃ alkyl;
R⁵ is H, deuterium, C₁-C₃ alkyl, F, or C₁;
Dn1 is 0, 1, 2 or 3; and
Dn2 is 0, 1 or 2,
wherein the Degron is covalently bonded to the Linker via

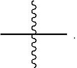

16. The bifunctional compound of claim 15, wherein X is C(O).

17. The bifunctional compound of claim 15, wherein Y is O.

18. The bifunctional compound of claim 17, wherein the Degron is of Formula D1a or D1b:

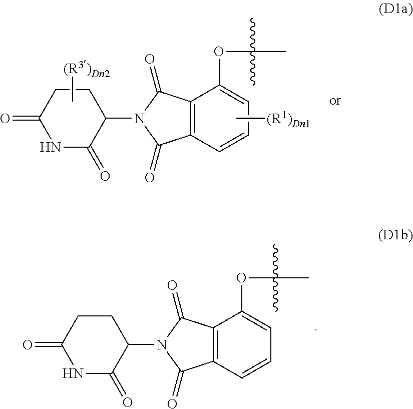

19. The bifunctional compound of claim 1, wherein the Degron is of Formula D2: 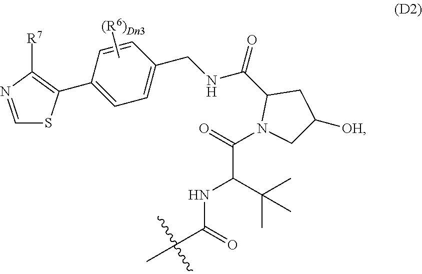
or an enantiomer, diastereomer, or stereoisomer thereof, wherein:
each R⁶ is independently C₁-C₃ alkyl;
Dn3 is 0, 1, 2, 3 or 4; and
R⁷ is C₁-C₃ alkyl,
wherein the Degron is covalently bonded to the Linker via

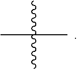

20. The bifunctional compound of claim 19, wherein R⁷ is methyl.

21. The bifunctional compound of claim 19, wherein the Degron is of Formula D2a or D2b:

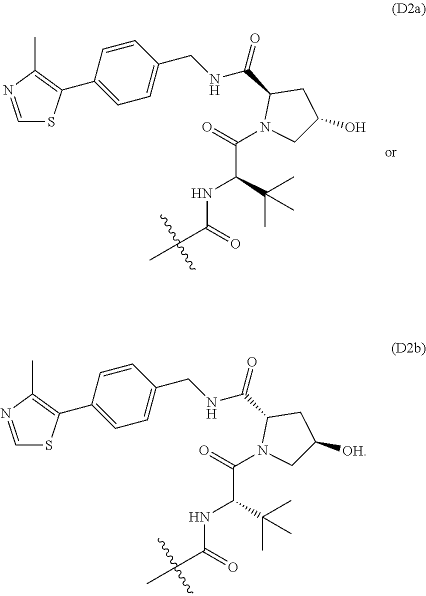 [SMILES: *C(=O)N[C@@H](C(=O)N1C[C@@H](O)C[C@@H]1C(=O)NCc1ccc(-c2scnc2C)cc1)C(C)(C)C ; *C(=O)N[C@H](C(=O)N1C[C@H](O)C[C@H]1C(=O)NCc1ccc(-c2scnc2C)cc1)C(C)(C)C]

22. A bifunctional compound of claim 1, wherein the compound is selected from

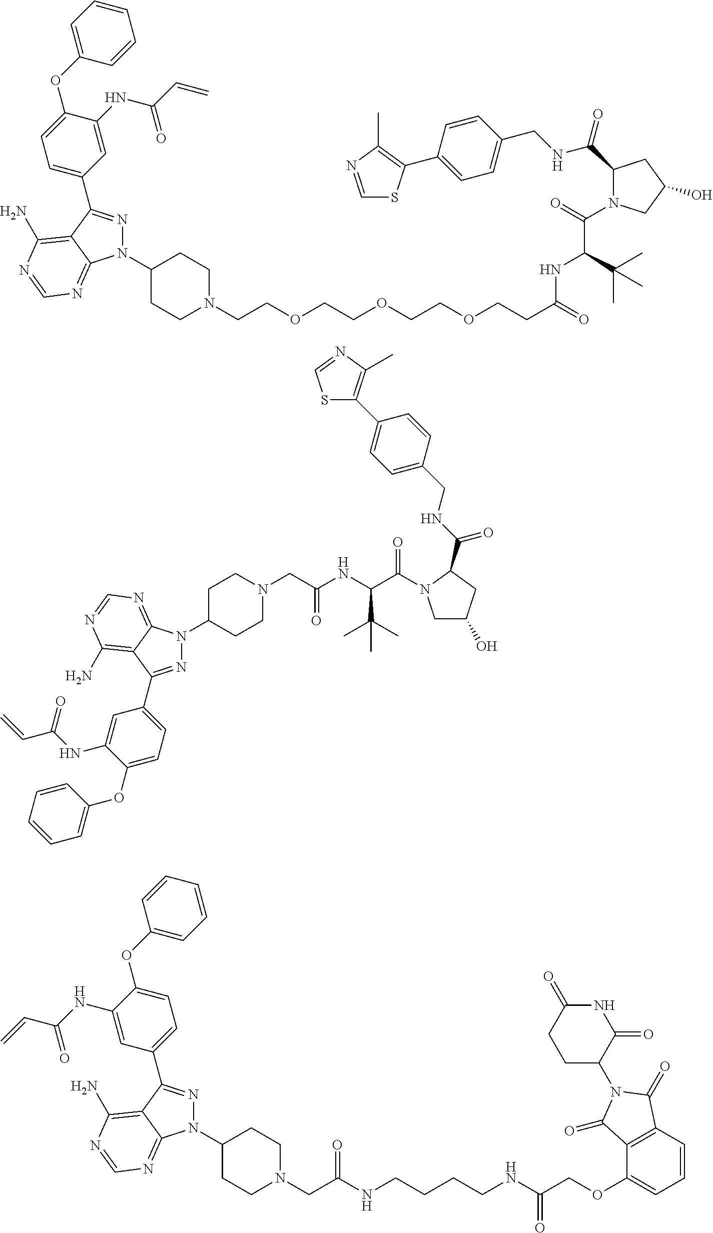

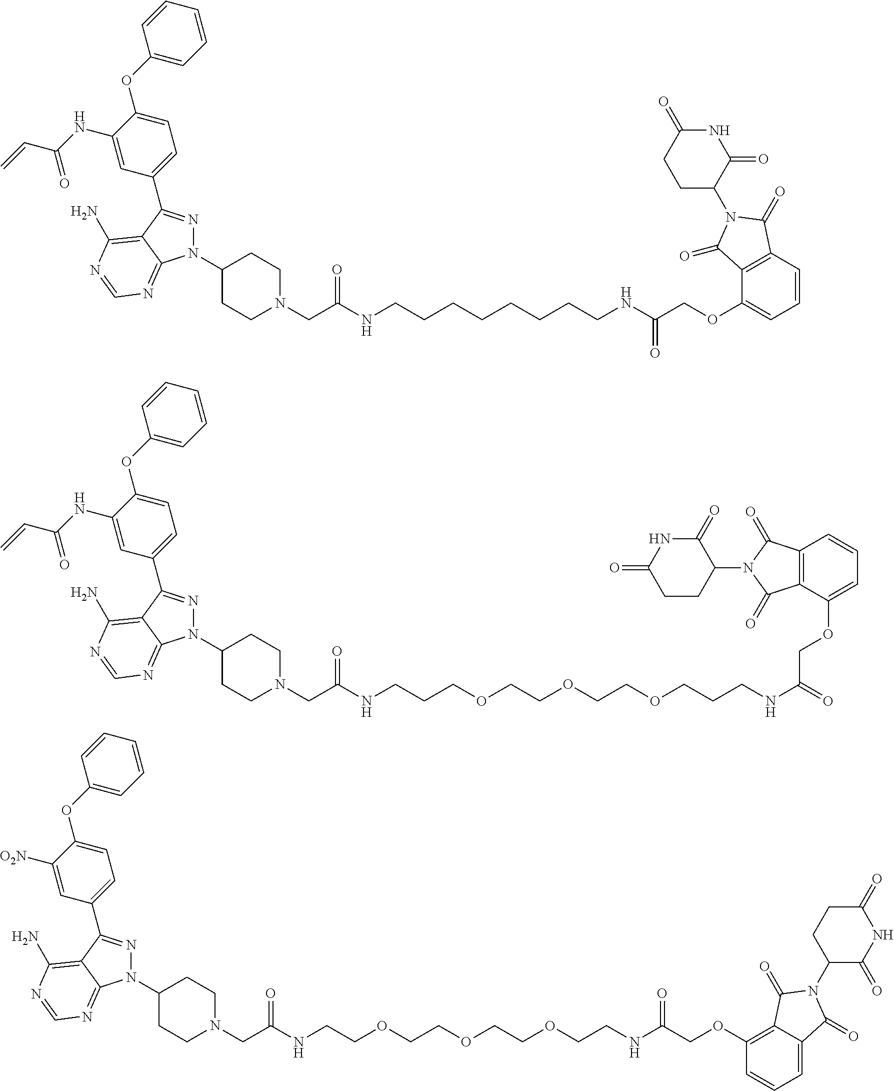 [SMILES: C=CC(=O)Nc1cc(-c2nn(C3CCN(CC(=O)NCCCCCCCCNC(=O)COc4cccc5c4C(=O)N(C4CCC(=O)NC4=O)C5=O)CC3)c3ncnc(N)c23)ccc1Oc1ccccc1 ; C=CC(=O)Nc1cc(-c2nn(C3CCN(CC(=O)NCCCOCCOCCOCCCNC(=O)COc4cccc5c4C(=O)N(C4CCC(=O)NC4=O)C5=O)CC3)c3ncnc(N)c23)ccc1Oc1ccccc1 ; Nc1ncnc2c1c(-c1ccc(Oc3ccccc3)c([N+](=O)[O-])c1)nn2C1CCN(CC(=O)NCCOCCOCCOCCNC(=O)COc2cccc3c2C(=O)N(C2CCC(=O)NC2=O)C3=O)CC1]

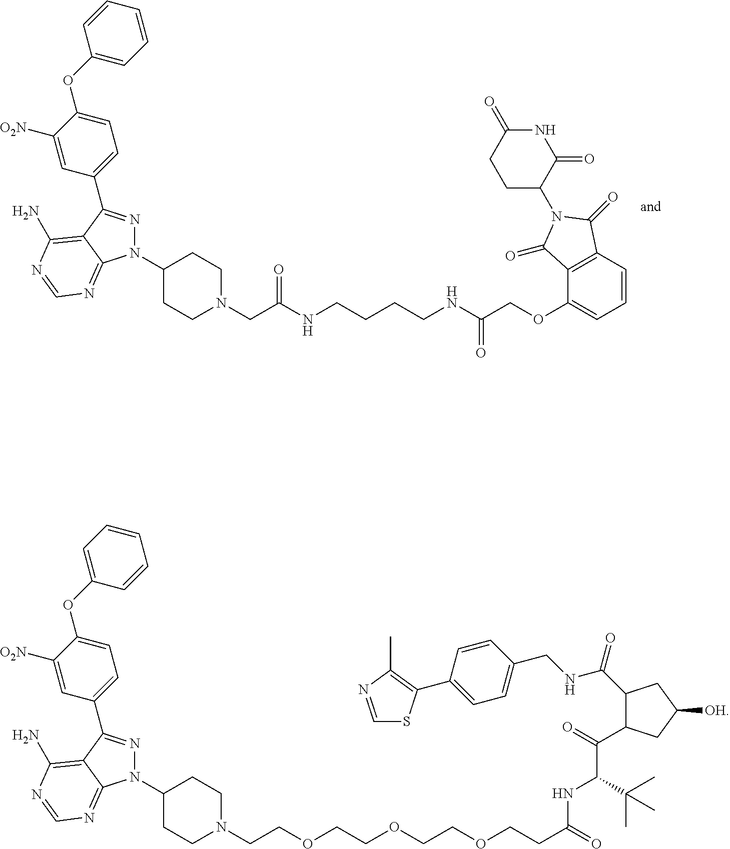 [SMILES: Nc1ncnc2c1c(-c1ccc(Oc3ccccc3)c([N+](=O)[O-])c1)nn2C1CCN(CC(=O)NCCCCNC(=O)COc2cccc3c2C(=O)N(C2CCC(=O)NC2=O)C3=O)CC1 ; Cc1ncsc1-c1ccc(CNC(=O)C2C[C@@H](O)CC2C(=O)[C@@H](NC(=O)CCOCCOCCOCCN2CCC(n3nc(-c4ccc(Oc5ccccc5)c([N+](=O)[O-])c4)c4c(N)ncnc43)CC2)C(C)(C)C)cc1]

23. A pharmaceutical composition comprising a therapeutically effective amount of the bifunctional compound of claim 1, or an enantiomer, diastereomer, stereoisomer, or pharmaceutically acceptable salt thereof, and a pharmaceutically acceptable carrier.

24. A method for modulating the amount of a HER family protein, comprising administering a therapeutically effective amount of the bifunctional compound of claim 1, or an enantiomer, diastereomer, stereoisomer, or pharmaceutically acceptable salt thereof, to a subject in need thereof.

25. A method for treating a disease or condition modulated by a HER family protein, comprising administering a therapeutically effective amount of the bifunctional compound of claim 1, or an enantiomer, diastereomer, stereoisomer, or pharmaceutically acceptable salt thereof, to a subject in need thereof.

26.-31. (canceled)
